(19) United States
(12) Patent Application Publication
(10) Pub. No.: US 2025/0092386 A1
(43) Pub. Date: Mar. 20, 2025

(54) ARTIFICIAL EXONIC BARCODE SYSTEM

(71) Applicant: The Curators of the University of Missouri, Columbia, MO (US)

(72) Inventors: Dongsheng Duan, Columbia, MO (US); Matthew J. Burke, Columbia, MO (US); Xiufang Pan, Columbia, MO (US); Yongping Yue, Columbia, MO (US); Shi-jie Chen, Columbia, MO (US); Jun Li, Columbia, MO (US)

(21) Appl. No.: 18/808,366

(22) Filed: Aug. 19, 2024

Related U.S. Application Data
(60) Provisional application No. 63/583,005, filed on Sep. 15, 2023.

(51) Int. Cl. C12N 15/10; C12N 15/86; C40B 40/06; G16B 30/10

(52) U.S. Cl. CPC C12N 15/1082; C12N 15/86; C40B 40/06; G16B 30/10; C12N 2750/14143

(57) ABSTRACT

The present disclosure is generally directed to an artificial exonic barcode system. The exonic barcodes comprise a nucleotide sequence comprising from 5′ to 3′ a 5′ barcode, an intron, and a 3′ barcode, and the disclosure is further directed to a library of these exonic barcodes. The disclosure also describes a method of generating the exonic barcode library and using the library of exonic barcodes in a method of screening for efficiency of transformation and/or expression of one or more genetic constructs in a subject. Primers and probes were also designed for validation of these exonic barcodes and corresponding methods.

### CROSS-REFERENCE TO RELATED APPLICATION(S)

This invention claims priority to U.S. Provisional Application Ser. No. 63/583,005, filed Sep. 15, 2023, which is incorporated by reference in its entirety.

### STATEMENT REGARDING FEDERALLY SPONSORED RESEARCH OR DEVELOPMENT

This invention was made with government support under NS090634 and NS131416 awarded by the National Institutes of Health. The government has certain rights in the invention.

### INCORPORATION OF SEQUENCE LISTING XML

A computer readable form of the Sequence Listing XML containing the file named “UMCO-H563US-17193-00128.xml,” which is 211,000 bytes in size and was created on Aug. 14, 2024, is provided herein and is herein incorporated by reference. This Sequence Listing consists of SEQ ID NOs: 1-236.

### FIELD OF DISCLOSURE

The present disclosure provides an artificial exonic barcode system that can be delivered with genetic constructs to differentiate between genome copies and transcript copies of the genetic construct in downstream evaluation methods such as real-time PCR, high throughput sequencing, conventional PCR, Southern blotting, Northern blotting, and in situ hybridization. For example, this can be used to evaluate the transduction and/or induction efficiency of AAV capsids in various tissue types. The present disclosure also provides a method of generating the artificial exonic barcode system.

### BACKGROUND OF DISCLOSURE

The treatment effect of gene therapy is achieved by delivering a beneficial DNA expression cassette to patients using a viral or nonviral vector. Vector selection is a major determining factor on whether gene therapy will ameliorate disease without inducing side effects. Often, there are a dozen candidate vectors to select from. The traditional approach compares these vectors side-by-side in a relevant animal model. This approach was tested by comparing 8 AAV capsids in a canine model for systemic muscle gene delivery. Great animal-to-animal and muscle-to-muscle differences were found, suggesting the traditional approach is unreliable. The short nucleotide (3 to 15 nucleotides) barcode system was developed in the last few years. Several groups have used this system to compare the transduction and expression of various AAV capsids using high-throughput sequencing and bioinformatic analysis. However, the short barcode system has many limitations, including (1) the data cannot be validated by a different method, (2) it is not suitable for in situ evaluation of the transduction and expression at the single-cell level in tissues, (3) the cDNA sequence and the gene sequence are identical, making it impossible to completely rule out DNA contamination in the cDNA preparation completely, (4) the bioinformatic tools influence the results, and (5) different algorithms may yield different outcomes. To overcome these limitations, an artificial exonic barcode system was developed.

### SUMMARY OF DISCLOSURE

The present disclosure provides an exonic barcode comprising a nucleotide sequence comprising, from 5′ to 3′, a 5′ barcode, an intron, and a 3′ barcode,
wherein the 5′ barcode is at least 50 bp long;
wherein the 3′ barcode is at least 50 bp long;
wherein at least one of the 5′ barcode and 3′ barcode is at least 150 bp long;
wherein the 5′ barcode and 3′ barcode have minimum homology with human, monkey, pig, dog, rabbit, mouse, and rat genomes and have minimum homology with each other;
wherein minimum homology is defined by a BLAST search E-value of greater than 0.05;
wherein the exonic barcode does not have alternative splice sites;
wherein the 5′ barcode and 3′ barcode each has no repeated sub-fragments longer than 6 nucleotides;
wherein the 5′ barcode and 3′ barcode each does not contain a target sequence of any restriction enzyme used in cloning the exonic barcode or any sequence identical to the target sequence except for one different nucleotide;
wherein the 5′ barcode and 3′ barcode each do not contain four identical nucleotides in a row;
wherein the 5′ barcode ends with a “CAG” nucleotide sequence and does not contain a “GGT” nucleotide sequence; and
wherein the 3′ barcode starts with a “G” nucleotide and does not contain an “AAG” nucleotide sequence.

The present disclosure further provides a library of exonic barcodes comprising two or more exonic barcodes as described elsewhere herein, wherein there are no duplicated fragments longer than eight nucleotides shared among any 5′ barcode, any 3′ barcode, and any 5′ barcode and 3′ barcode.

The present disclosure is also directed to a method of generating an exonic barcode library, the method comprising:
a) independently generating a 5′ DNA fragment library and a 3′ DNA fragment library each comprising at least 200,000 20-nucleotide-long random DNA fragments;
wherein each random DNA fragment in the 5′ DNA fragment library and the 3′ DNA fragment library has no repeated sub-fragment longer than 6 nucleotides, each fragment does not contain a target sequence of any restriction enzyme to be used in cloning the exonic barcode library or any sequence identical to the target sequence except for one different nucleotide, and each fragment does not contain four identical nucleotides in a row;
wherein each random fragment in the 5′ DNA fragment library does not contain the sequence “GGT;”
wherein each fragment in the 3′ DNA fragment library does not contain the sequence “AGG”;
b) generating a refined 5′ DNA fragment library by removing DNA fragments from the 5′ DNA fragment library that have a maximum aligned identical sequence length of greater than 21 nucleotides with human and/or dog genomes or that share sequence fragment lengths of greater than 8 nucleotides with any other fragments of the 5′ and/or 3′ DNA fragment libraries; and
generating a refined 3′ DNA fragment library by removing DNA fragments from the 3′ DNA fragment library that have a maximum aligned identical sequence length of greater than 18 nucleotides with human and/or dog genomes or that share sequence fragment lengths of greater than 8 nucleotides with any other fragments of the 5′ and/or 3′ DNA fragment libraries;
c) generating a 5′ exonic barcode library comprising at least 500,000 150 nucleotide-long 5′ barcodes by combining eight 20-nucleotide-long random DNA fragments from the refined 5′ DNA fragment library and removing the last 10 nucleotides and generating a 3′ exonic barcode library comprising at least 500,000 50-nucleotide-long 3′ barcodes by combining three 20-nucleotide-long random DNA fragments from the refined 3′ DNA fragment library and removing the last 10 nucleotides;
wherein each barcode of the 5′ exonic barcode library or the 3′ exonic barcode library has no repeated sub-fragment longer than 6 nucleotides, the 5′ barcode and 3′ barcode each do not contain a target sequence of any restriction enzyme used in cloning the exonic barcode or any sequence identical to the target sequence except for one different nucleotide, and each barcode does not contain four identical nucleotides in a row;
wherein each barcode in the 5′ exonic barcode library ends with a “CAG” nucleotide sequence and does not contain a “GGT” nucleotide sequence;
wherein each barcode in the 3′ exonic barcode library starts with a “G” nucleotide and does not contain an “AAG” nucleotide sequence;
d) generating a refined 5′ exonic barcode library and a refined 3′ exonic barcode library by removing any barcodes that have a maximum aligned identical sequence length of greater than 8 with any other barcode in either library and removing any barcodes that share homology with the human, monkey, pig, dog, rabbit, mouse, and/or rat genomes, wherein sharing homology is defined by a BLAST search E-value of 0.05 or less; and
e) generating the exonic barcode library comprising exonic barcodes, wherein each exonic barcode is generated by combining, from 5′ to 3′, one barcode from the refined 5′ exonic barcode library, an intron, and one barcode from the refined 3′ exonic barcode library, and wherein any exonic barcode that comprises an alternative splice site is removed from the exonic barcode library.

The present disclosure is also directed to a method of screening for efficiency of transformation and/or expression of one or more genetic constructs in a subject, the method comprising:
a) transforming the one or more genetic constructs into the subject, wherein each of the one or more genetic constructs comprises a nucleotide sequence encoding a different protein of interest conjugated to a different exonic barcode as described elsewhere herein;
b) harvesting cells from the subject;
c) performing on the cells one or more methods selected from the group consisting of real-time PCR, high-throughput sequencing, conventional PCR, Southern blotting, Northern blotting, and in situ hybridization; and
d) evaluating the one or more methods for the relative amounts of genome copies and/or transcript copies of the one or more genetic constructs to determine the efficiency of transformation and/or expression.

The present disclosure is further directed to a primer comprising a nucleotide sequence of any one of SEQ ID NO: 146-159, 174-201, and 216-229; a real-time PCR primer comprising a nucleotide sequence of any one of SEQ ID NO: 146-159, 174-201, and 216-229, a fluorophore, and a quencher; and an in situ hybridization probe comprising a nucleotide sequence of any one of SEQ ID NO: 160-173 and 202-215 and a label.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 depicts a comparison of muscle transduction efficiency of 8 different AAV capsids in canines.

FIG. 2 depicts a cartoon illustration of the artificial exonic barcode system.

FIGS. 3A-3D depict strategies to evaluate AAV transduction and expression using the artificial exonic barcode system. FIG. 3A depicts how AAV transduction and expression can be studied using TaqMan™ PCR. Arrows refer to PCR primers. Dotted lines refer to the TaqMan™ PCR probe. FIG. 3B depicts how AAV transduction and expression can be studied using high throughput sequencing from multiple directions. Arrows refer to sequencing primers. FIG. 3C depicts how AAV transduction and expression can be studied using conventional PCR. Arrows refer to PCR primers. FIG. 3D depicts how AAV transduction and expression can be studied using Southern blot/Northern blot and DNAscope™/RNAscope™/Basescope™ techniques. Dumbbell lines refer to probes for Southern blot/Northern blot, DNAscope™, RNAscope™, and Basescope™.

FIG. 4 depicts conserved splicing donor and acceptor signals (dotted boxes).

FIG. 5 depicts a flowchart illustration of the bioinformatics design of the exonic barcodes. * indicates sequence similarities of the exonic barcodes were compared with the human, monkey, dog, pig, rabbit, rat, and mouse genomes.

FIGS. 6A-6F depict a Blast search of 5′-exonic barcode 1. FIG. 6A depicts a summary of the search results. FIG. 6B depicts an illustration of the search results. The line on the top of the figure represents the exonic barcode. The shorter lines represent the alignment of the human/dog genome sequence with the barcode sequence.

FIGS. 6C-6F each depict detailed alignment information of the barcode sequence with the genome sequence and include SEQ ID NOs: 230-235.

FIG. 7 depicts examples of Blast search of the TaqMan™ PCR primers and probes.

FIG. 8 depicts the plasmid numbers associated with each barcode, as well as a plasmid with all 14 barcodes in one plasmid.

FIGS. 9A-9C depict a strategy to evaluate cross-reactivity of vector genome TaqMan™ PCR primers and probes. FIG. 9A depicts a cartoon illustration of the barcode-1 and the primer/probe set to quantify the vector genome copy number of barcode-1. FIG. 9B depicts three PCR reactions that were used to check the specificity of the primer/probe set for barcode-1. In reaction 1, only the barcode-1 plasmid (XP149) was used as the template. In reaction 2, the all-in-one plasmid (XP249) was used as the template. In reaction 3, a mixture of all 14 barcode plasmids was used as the template. FIG. 9C depicts an example of amplification plots for one barcode at one concentration. The same reaction was carried out for each barcode at 8 different concentrations (2×10², 2×10³, 2×10⁴, 2×10⁵, 2×10⁶, 2×10⁷, 2×10⁸, and 2×10⁹ copies/plasmid). The same set of reactions was carried out for all 14 barcodes with the results shown in FIG. 10.

FIG. 10 depicts an evaluation of the specificity of the primers and probes designed to quantify the vector genome copy number (the efficiency of AAV transduction, i.e. the efficiency of delivering the AAV genome to the target tissue). Three sets of PCR reactions were carried out for each barcode using the barcode-specific primer/probe set at 8 different template concentrations (2×10², 2×10³, 2×10⁴, 2×10⁵, 2×10⁶, 2×10⁷, 2×10⁸, and 2×10⁹ copies/plasmid). In the first reaction, the plasmid corresponding to the primer/probe set was used as the template (Barcode-). In the second reaction, an all-in-one plasmid was used as the template (All-in-one). In the third reaction, a mixture of all 14 plasmids was used as the template (Mixture).

FIGS. 11A and 11B depict an additional evaluation of the specificity of TaqMan™ PCR primers and probes designed to quantify the vector genome copy number. Two independent sets of PCR reactions were performed. The Ct values of these PCR reactions are shown in FIG. 11A and FIG. 11B. In these PCR reactions, 1×10e5 copies of the linearized plasmid were used as the template. The template barcode plasmid used in each reaction was shown in the top row. The primer/probe set used in each reaction was marked in the far-left column. NTC, no template control; UD, undetectable.

FIG. 12 depicts a linear regression analysis for PCR reactions that used the all-in-one plasmid as the template but a barcode-specific primer/probe set in each PCR.

FIG. 13 depicts a series of plasmids to mimic the cDNA sequence of each barcode.

FIGS. 14A-14C depict a strategy to evaluate cross-reactivity of transcript TaqMan™ PCR primers and probes. FIG. 14A depicts a cartoon illustration of the barcode-5 and the primer/probe set to quantify the transcript copy number of barcode-5. FIG. 14B depicts three PCR reactions were used to check the specificity of the primer/probe set for barcode-5. In reaction 1, only the barcode-5 plasmid was used as the template. In reaction 2, an all-in-one plasmid was used as the template. In reaction 3, a mixture of all 14 individual barcode plasmids was used as the template. FIG. 14C depicts an example of amplification plots for one barcode at one concentration. The same reaction was carried out for each barcode at 8 different concentrations (2×10², 2×10³, 2×10⁴, 2×10⁵, 2×10⁶, 2×10⁷, 2×10⁸, and 2×10⁹ copies/plasmid). The same set of reactions was carried out for all 14 barcodes.

FIG. 15 depicts an evaluation of the specificity of the primers and probes designed to quantify the copy number of the vector transcript (the efficiency of AAV-mediated transgene expression). Three sets of PCR reactions were carried out for each barcode cDNA using the barcode-specific primer/probe set at 8 different template concentrations (2×10², 2×10³, 2×10⁴, 2×10⁵, 2×10⁶, 2×10⁷, 2×10⁸, and 2×10⁹ copies/plasmid). The template plasmids used in this experiment do not contain introns (see FIG. 13). In the first reaction, the plasmid corresponding to the primer/probe set was used as the template (Barcode-). In the second reaction, an all-in-one plasmid was used as the template (All-in-one). In the third reaction, a mixture of all 14 plasmids was used as the template (Mixture).

FIG. 16 depicts a linear regression analysis for PCR reactions that used the cDNA all-in-one plasmid as the template but a barcode-specific primer/probe set in each PCR.

FIG. 17 depicts testing for cross-reactivity among different primer/probe sets when AAV virus was used as the template. NTC, no template control; UD, undetected.

FIG. 18 depicts a vector genome copy number quantification. AAVB1 carries barcode-1, AAV2 carries barcode-3, AAV8 carries barcode-4, AAV9 carries barcode-6, AAVrh74 carries barcode-7, AAVMYO carries barcode-8, AAV-S1P1 carries barcode-10, AAV-S10P1 carries barcode-11, AAV-NP22 carries barcode-12, AAV-NP66 carries barcode-13, and AAV-KP1 carries barcode-14.

FIG. 19 depicts a transcript copy number quantification. AAVB1 carries barcode-1, AAV2 carries barcode-3, AAV8 carries barcode-4, AAV9 carries barcode-6, AAVrh74 carries barcode-7, AAVMYO carries barcode-8, AAV-S1P1 carries barcode-10, AAV-S10P1 carries barcode-11, AAV-NP22 carries barcode-12, AAV-NP66 carries barcode-13, and AAV-KP1 carries barcode-14.

FIG. 20 depicts a vector genome copy number quantification. AAVB1 carries barcode-1, AAV2 carries barcode-3, AAV8 carries barcode-4, AAV9 carries barcode-6, AAVrh74 carries barcode-7, AAVMYO carries barcode-8, AAV-S1P1 carries barcode-10, AAV-S10P1 carries barcode-11, AAV-NP22 carries barcode-12, AAV-NP66 carries barcode-13, and AAV-KP1 carries barcode-14.

FIG. 21 depicts a transcript copy number quantification. AAVB1 carries barcode-1, AAV2 carries barcode-3, AAV8 carries barcode-4, AAV9 carries barcode-6, AAVrh74 carries barcode-7, AAVMYO carries barcode-8, AAV-S1P1 carries barcode-10, AAV-S10P1 carries 22 barcode-11, AAV-NP22 carries barcode-12, AAV-NP66 carries barcode-13, and AAV-KP1 carries barcode-14.

FIG. 22 depicts a comparison of AAV transduction (vector genome copy number) and expression (transcript copy number) in dogs. AAVB1 carries barcode-1, AAV2 carries barcode-3, AAV8 carries barcode-4, AAV9 carries barcode-6, AAVrh74 carries barcode-7, AAVMYO carries barcode-8, AAV-S1P1 carries barcode-10, AAV-S10P1 carries barcode-11, AAV-NP22 carries barcode-12, AAV-NP66 carries barcode-13, and AAV-KP1 carries barcode-14.

FIG. 23 depicts a summary of transduction (vector genome copy number) and expression (transcript copy number) data from mdx4cv mice and dogs. AAV8, AAV9, and AAVrh74 are used in clinical trials. AAVMYO is the best liver-detargeted myotropic capsid. AAV-KP1 is a liver tropic capsid.

### DETAILED DESCRIPTION OF INVENTION

This disclosure describes an exonic barcode comprising a nucleotide sequence comprising, from 5′ to 3′, a 5′ barcode, an intron, and a 3′ barcode,
wherein the 5′ barcode is at least 50 bp long;
wherein the 3′ barcode is at least 50 bp long;
wherein at least one of the 5′ barcode and 3′ barcode is at least 150 bp long;
wherein the 5′ barcode and 3′ barcode have minimum homology with human, monkey, pig, dog, rabbit, mouse, and rat genomes and have minimum homology with each other;
wherein minimum homology is defined by a BLAST search E-value of greater than 0.05;
wherein the exonic barcode does not have alternative splice sites;
wherein the 5′ barcode and 3′ barcode each has no repeated sub-fragments longer than 6 nucleotides;
wherein the 5′ barcode and 3′ barcode each does not contain a target sequence of any restriction enzyme used in cloning the exonic barcode or any sequence identical to the target sequence except for one different nucleotide;
wherein the 5′ barcode and 3′ barcode each do not contain four identical nucleotides in a row;
wherein the 5′ barcode ends with a “CAG” nucleotide sequence and does not contain a “GGT” nucleotide sequence; and
wherein the 3′ barcode starts with a “G” nucleotide and does not contain an “AAG” nucleotide sequence.

The intron can be any intron known in the art. The intron can be a pCI intron. In particular, the intron can be a pCI intron of SEQ ID NO: 236.

The 5′ barcode can have a maximum aligned identical sequence length with the human and/or dog genome of equal to or less than 21. The 3′ barcode can have a maximum aligned identical sequence length with the human and/or dog genome of equal to or less than 18. The 5′ barcode and 3′ barcode can have no identical sequence fragments equal to or greater than 8 nucleotides. The nucleotide sequence of the exonic barcode can be at least 300 nucleotides long. The nucleotide sequence can comprise any one of SEQ ID NO: 31 AND 33-45.

The human genome can be a Homo sapiens genome. The monkey genome can be a Macaca mulatta genome. The pig genome can be a Sus scrofa genome. The dog genome can be a Canis lupus familiaris genome. The rabbit genome can be a Oryctolagus cuniculus genome. The mouse genome can be a Mus musculus genome. The rat genome can be a Rattus norvegicus genome.

The present disclosure is further directed to a synthetic reporter gene comprising a nucleotide sequence comprising a reporter coding sequence and an exonic barcode as described elsewhere herein. The reporter can be GFP, EGFP, RFP, BFP, YFP, Luciferase, or any other reporter known in the art.

The present disclosure is also directed to a library of exonic barcodes comprising two or more exonic barcodes as described elsewhere herein, wherein there are no duplicated fragments longer than eight nucleotides shared among any 5′ barcode, any 3′ barcode, and any 5′ barcode and 3′ barcode.

The present disclosure is further directed to a method of generating an exonic barcode library, the method comprising:
a) independently generating a 5′ DNA fragment library and a 3′ DNA fragment library each comprising at least 200,000 20-nucleotide-long random DNA fragments;
wherein each random DNA fragment in the 5′ DNA fragment library and the 3′ DNA fragment library has no repeated sub-fragment longer than 6 nucleotides, each fragment does not contain a target sequence of any restriction enzyme to be used in cloning the exonic barcode library or any sequence identical to the target sequence except for one different nucleotide, and each fragment does not contain four identical nucleotides in a row;
wherein each random fragment in the 5′ DNA fragment library does not contain the sequence “GGT;”
wherein each fragment in the 3′ DNA fragment library does not contain the sequence “AGG”;
b) generating a refined 5′ DNA fragment library by removing DNA fragments from the 5′ DNA fragment library that have a maximum aligned identical sequence length of greater than 21 nucleotides with human and/or dog genomes or that share sequence fragment lengths of greater than 8 nucleotides with any other fragments of the 5′ and/or 3′ DNA fragment libraries; and
generating a refined 3′ DNA fragment library by removing DNA fragments from the 3′ DNA fragment library that have a maximum aligned identical sequence length of greater than 18 nucleotides with human and/or dog genomes or that share sequence fragment lengths of greater than 8 nucleotides with any other fragments of the 5′ and/or 3′ DNA fragment libraries;
c) generating a 5′ exonic barcode library comprising at least 500,000 150 nucleotide-long 5′ barcodes by combining eight 20-nucleotide-long random DNA fragments from the refined 5′ DNA fragment library and removing the last 10 nucleotides and generating a 3′ exonic barcode library comprising at least 500,000 50-nucleotide-long 3′ barcodes by combining three 20-nucleotide-long random DNA fragments from the refined 3′ DNA fragment library and removing the last 10 nucleotides;
wherein each barcode of the 5′ exonic barcode library or the 3′ exonic barcode library has no repeated sub-fragment longer than 6 nucleotides, the 5′ barcode and 3′ barcode each do not contain a target sequence of any restriction enzyme used in cloning the exonic barcode or any sequence identical to the target sequence except for one different nucleotide, and each barcode does not contain four identical nucleotides in a row;
wherein each barcode in the 5′ exonic barcode library ends with a “CAG” nucleotide sequence and does not contain a “GGT” nucleotide sequence;
wherein each barcode in the 3′ exonic barcode library starts with a “G” nucleotide and does not contain an “AAG” nucleotide sequence;
d) generating a refined 5′ exonic barcode library and a refined 3′ exonic barcode library by removing any barcodes that have a maximum aligned identical sequence length of greater than 8 with any other barcode in either library and removing any barcodes that share homology with the human, monkey, pig, dog, rabbit, mouse, and/or rat genomes, wherein sharing homology is defined by a BLAST search E-value of 0.05 or less; and
e) generating the exonic barcode library comprising exonic barcodes, wherein each exonic barcode is generated by combining, from 5′ to 3′, one barcode from the refined 5′ exonic barcode library, an intron, and one barcode from the refined 3′ exonic barcode library, and wherein any exonic barcode that comprises an alternative splice site is removed from the exonic barcode library.

The exonic barcode can have a GC content of about 50% to about 60%. The 5′ barcode and 3′ barcode can each not contain “TTAATTAA,” “GCTAGC,” or any sequence identical to “TTAATTAA” or “GCTAGC” except for one different nucleotide. Each barcode from the 5′ exonic barcode library and the refined 3′ exonic barcode library can be used at most once in generating the exonic barcodes of the exonic barcode library in step e). Step d) can comprise removing any barcode in the 5′ exonic barcode library that has a maximum aligned identical sequence length with the human and/or dog genome of greater than 21. Step d) can comprise removing any barcode in the 3′ exonic barcode library that has a maximum aligned identical sequence length with the human and/or dog genome of greater than 18.

The human genome can be a Homo sapiens genome. The monkey genome can be a Macaca mulatta genome. The pig genome can be a Sus scrofa genome. The dog genome can be a Canis lupus familiaris genome. The rabbit genome can be a Oryctolagus cuniculus genome. The mouse genome can be a Mus musculus genome. The rat genome can be a Rattus norvegicus genome.

The present disclosure is also directed to a method of screening for efficiency of transformation and/or expression of one or more genetic constructs in a subject, the method comprising:
a) transforming the one or more genetic constructs into the subject, wherein each of the one or more genetic constructs comprises a nucleotide sequence encoding a different protein of interest conjugated to a different exonic barcode as described elsewhere herein;
b) harvesting cells from the subject;
c) performing on the cells one or more methods selected from the group consisting of real-time PCR, high-throughput sequencing, conventional PCR, Southern blotting, Northern blotting, and in situ hybridization; and
d) evaluating the one or more methods for the relative amounts of genome copies and/or transcript copies of the one or more genetic constructs to determine the efficiency of transformation and/or expression.

The transformation can be any transformation method known in the art. The transformation can be a stable integration or via transfection or a virus. The virus can be AAV or any virus used in the art for transformation. The protein of interest of the one or more genetic constructs can each comprise a different AAV capsid. The subject can be a human, a non-human primate, pig, canine, rabbit, mouse, rat, or a cell line thereof. The one or more genetic constructs comprise up to 14 genetic constructs.

The method of screening for efficiency of transformation and/or expression of one or more genetic constructs in a subject can further comprise harvesting cells from more than one tissue of the subject in step b) and performing steps c) and d) separately on the cells from each tissue to screen for efficiency of transformation and/or expression separately in each tissue. The more than one tissue can comprise at least two tissues selected from the list consisting of heart, retina, brain, spinal cord, kidney, lung, muscle, and liver tissue. More specifically, the more than one tissue can comprise muscle tissue and liver tissue.

The present disclosure is further directed to a primer comprising a nucleotide sequence of any one of SEQ ID NO: 146-159, 174-201, and 216-229; a real-time PCR primer comprising a nucleotide sequence of any one of SEQ ID NO: 146-159, 174-201, and 216-229, a fluorophore, and a quencher; and an in situ hybridization probe comprising a nucleotide sequence of any one of SEQ ID NO: 160-173 and 202-215 and a label.

As used in this application, including the appended claims, the singular forms “a,” “an,” and “the” include plural references unless the content clearly dictates otherwise, and are used interchangeably with “at least one” and “one or more.”

The invention will be further described in the following examples, which do not limit the scope of the invention described in the claims.

### EXAMPLES

It is to be understood that while the invention has been described in conjunction with the detailed description thereof, the preceding description is intended to illustrate and not limit the scope of the invention, which is defined by the scope of the appended claims. Other aspects, advantages, and modifications are within the scope of the following claims.

To overcome the limitations of previous methods to compare transduction and expression of various AAV capsids, an artificial exonic barcode system was developed. This system is based on 14 pairs of carefully designed artificial exons distinctive from the genome sequence of commonly studied species including humans, non-human primates, dogs, pigs, mice, rats, and rabbits. Exonic barcode-specific TaqMan™ qPCR assays for quantifying the vector genome and the transcript copy number were also designed and validated. 11 AAV capsids were screened using this system in a mouse model of Duchenne muscular dystrophy and in canines. These results are highly consistent with the literature. The exonic barcode system described in this disclosure is highly advantageous for identifying the best viral or nonviral vectors for gene therapy. This is detailed in the examples below.

### Example 1: Test Various Muscle Tropic AAV Capsids in the Canine Model

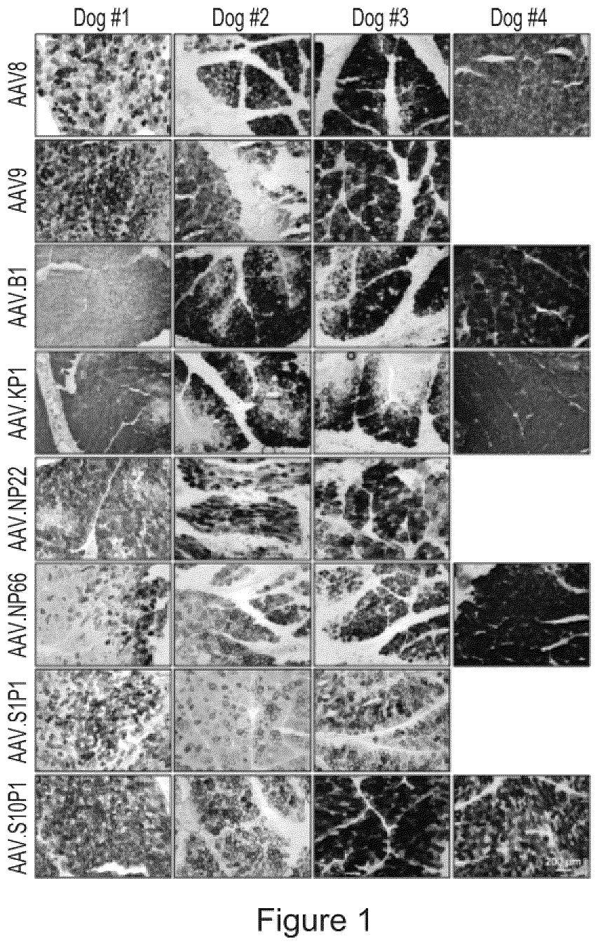

Traditionally, comparing the tissue tropism of different AAV serotypes was performed by delivering individual serotype AAV vector to the target tissue and then quantifying transgene expression. This approach was used in this first study. Specifically, 8 different AAV capsids (AAV8, AAV9, AAV.B1, AAV.KP1, AAV.NP22, AAV.NP66, AAV.S1P1, and AAV.S10P1) were tested in four 4-month-old normal dogs by local injection in various muscles [right and left extensor carpi ulnaris (ECU,) right and left flexor carpi ulnaris (FCU), right and left cranial tibialis (CT), and right and left semitendinosus (ST)] at the dose of 1×10¹¹ vg/muscle/AAV in a volume of 500 μl/muscle/AAV (FIG. 1). The same expression cassette (vector genome) was packaged in all AAV capsids. In this cassette, the expression of the heat-resistant human placental alkaline phosphatase (AP) gene was regulated by the Rous sarcoma virus (RSV) promoter and simian virus 40 (SV40) polyadenylation signal.

Two weeks after injection, animals were euthanized, and muscles were harvested. AAV-mediated expression was examined by histochemical staining for AP activity. Intriguingly, significant differences were found among different dogs and different muscles. This made it impossible to reach a solid conclusion on the transduction efficiency of various AAV capsids that were studied. It is suspected that this outcome was likely attributed to the differences in fiber type composition of different muscles and minor differences in injection techniques in each muscle, and individual variance of the experimental animals.

### Example 2: Development of an Artificial Exonic Barcode System to Study AAV Tropism

### Strategy Overview

The dog study suggests that the traditional AAV tropism comparison method cannot meet the need of large animal studies. To overcome this hurdle, the transduction efficiency of different AAV capsids must be compared in the same muscle of the same animal. This has been achieved by many groups in the last couple of years with barcoded AAV vectors. Specifically, a 3 to 15-nucleotide-long barcode is included in the AAV genome. Each barcoded AAV genome is packaged in a specific AAV variant. Barcode-tagged AAV vectors were mixed and delivered to the target tissue. AAV biodistribution and expression were then determined using high throughput sequencing of DNA and cDNA extracted from the target tissue, followed by bioinformatic analysis. Despite its widespread use, this method has many inherent limitations. First, DNA and cDNA share an identical barcode. Any contamination of DNA in the cDNA preparation may alter expression data. Second, this approach heavily depends on bioinformatic analysis. Differences in the analytic algorithm may yield different results. Third, the results cannot be validated by a different method. Fourth, it cannot reveal subcellular localization (spatial information) of vector transduction and transgene expression.

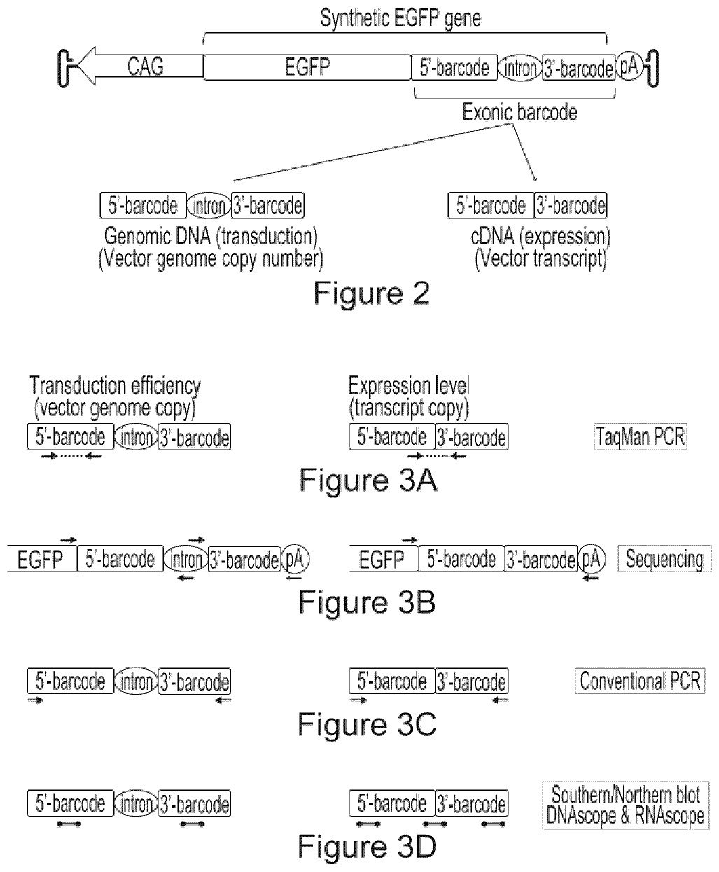

To overcome these limitations, an artificial exonic barcode system was developed. Specifically, a series of unique intron-containing synthetic EGFP genes were engineered. Each synthetic EGFP gene carries a ˜300 bp unique DNA fragment as the barcode. This system allows one to readily distinguish the cDNA from the genomic DNA because the intron is spliced out in the cDNA (FIG. 2). This system has several advantages. First, vector transduction (the amount of the vector genome in tissue) and transgene expression can be quantified by TaqMan™ PCR using the barcode-specific probe and primers. Second, vector transduction and transgene expression can be validated using high-throughput sequencing from multiple directions. Third, vector transduction and transgene expression can be further validated using barcode-specific conventional PCR. Fourth, vector transduction and transgene expression can also be validated by Southern and Northern blot, respectively, using the barcode-specific probe. Fifth, the cellular and spatial localization of the vector genome and the expression of the transgene can be determined at the single cell resolution by in situ hybridization with the DNAscope™/RNAscope™/Basescope™ techniques using the barcode-specific probe (FIG. 3A-3D).

In this study, the length of the 5′-exonic barcode was defined as 150 bp, and the length of the 3′-exonic barcode was defined as 50 bp. The synthetic intron from pCI (Promega, Madison, WI) was used as the intron in the synthetic EGFP gene. It is a β-globin/IgG chimeric intron of small size. The synthetic intron pCI has the sequence:

(SEQ ID NO: 236)
GTAAGTATCAAGGTTACAAGACAGGTTTAAGGAGACCAATAGAAACTG
GGCTTGTCGAGACAGAGAAGACTCTTGCGTTTCTGATAGGCACCTATT
GGTCTTACTGACATCCACTTTGCCTTTCTCTCCACAG

### Challenges in the Design of Exonic Barcodes

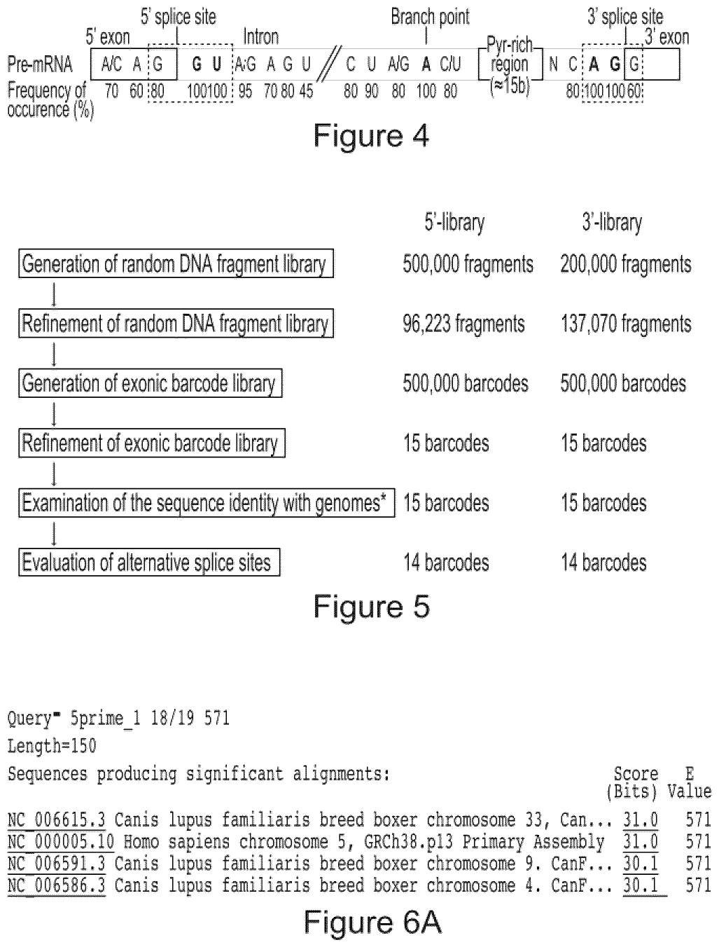

There are many challenges in designing exonic barcodes. First is the size of the exonic barcode. The conventional barcode is 3 to 15 nucleotides. For the exonic barcode, it is envisioned to be at least 50 bp on either side of the intron to meet the needs of various applications. One side of the barcode is also envisioned to be at least 100 bp to facilitate subsequent TaqMan™ PCR analysis. Second, the barcode sequence should have minimum overlap with the human genome sequence and the genome sequences of commonly used animal models (such as monkey, pig, dog, rabbit, mouse, and rat). In other words, there should be minimum homology between the barcode and the genome sequence. Third, the barcode sequence should not overlap with each other. Fourth, the barcode sequences should have similar GC content. Fifth, the 5′-barcode should not contain the conserved splicing donor signal (GGT), and the 3′-barcode should not contain the conserved splicing acceptor signal (AGG) (FIG. 4).

### Bioinformatic Design of Exonic Barcodes

To generate robust 5′ and 3′-exonic barcodes, a stepwise approach was taken (FIG. 5). First, two independent libraries of 20 nucleotide-long random DNA fragments were generated. Second, the DNA fragment libraries were filtered by pairwise sequence alignment, and sequences were removed that share high homology with the human and dog genomes. Third, two independent libraries for the 150-bp 5′-exonic barcode and the 50-bp 3′-exonic barcode were generated. Fourth, the exonic barcode libraries were filtered by pairwise sequence alignment to remove barcodes that share homology within two libraries or with the human and dog genomes. Fifth, sequence homology was cross-checked between the designed exonic barcodes and the mouse, rat, monkey, pig, and rabbit genomes. Sixth, the exonic barcodes were further narrowed using the alternative splice site predictor (ASSP) (Wang & Marin, 2006).

### Generation of the Random Short DNA Fragment Libraries

A custom-made algorithm (programmed with Python) was used to generate two random DNA fragment libraries called the 5′-fragment library and the 3′-fragment library.

```
Python Algorithm:
import sys
import random
import numpy as np
def generate_sequence(seq_length,gc_ratio,which_prime):
  gc_num = int(seq_length * gc_ratio)
  non_gc_num = seq_length − gc_num
  seq = “
  for i in range(gc_num):
    if random.random() < 0.5:
      seq += ‘G’
  else:
    seq += ‘C’
  for i in range(non_gc_num):
    if random.random() < 0.5:
    seq += ‘A’
  else:
    seq += ‘T’
  seq = list(seq)
  for i in range(50):
    random.shuffle(seq)
  seq = “”.join(seq)
  seq = filter_enzyme_and_splicing(seq,which_prime)
  return seq
enzyme_list = [‘TTAATTAA’, ‘ATAATTAA’, ‘CTAATTAA’, ‘GTAATTAA’,
‘TAAATTAA’, ‘TCAATTAA’, ‘TGAATTAA’, ‘TTTATTAA’, ‘TTCATTAA’,
‘TTGATTAA’, ‘TTATTTAA’, ‘TTACTTAA’, ‘TTAGTTAA’, ‘TTAAATAA’,
‘TTAACTAA’, ‘TTAAGTAA’, ‘TTAATAAA’, ‘TTAATCAA’, ‘TTAATGAA’,
‘TTAATTTA’, ‘TTAATTCA’, ‘TTAATTGA’, ‘TTAATTAT’, ‘TTAATTAC’,
‘TTAATTAG’, ‘GCTAGC’, ‘ACTAGC’, ‘TCTAGC’, ‘CCTAGC’, ‘GATAGC’, ‘GTTAGC’,
‘GGTAGC’, ‘GCAAGC’, ‘GCCAGC’, ‘GCGAGC’, ‘GCTTGC’, ‘GCTCGC’, ‘GCTGGC’,
‘GCTAAC’, ‘GCTATC’, ‘GCTACC’, ‘GCTAGA’, ‘GCTAGT’,
‘GCTAGG’,‘AAAA’, ‘GGGG’,‘TTTT’,‘CCCC’]
def has_enzyme(sequence):
  for enzyme in enzyme_list:
if enzyme in sequence:
  return True
  return False
def has_same_substr_within(s):
  K = 6
  fragments = []
  for i in range(len(s)−K+1):
    fragments.append(s[i:(i+K)])
  num_frag = len(fragments)
  for i in range(num_frag):
    for j in range(i+1,num_frag):
      if fragments[i] == fragments[j]:
        return True
  return False
def replace_splicing_signal(sequence,which_prime):
    if which_prime == ‘5prime’:
      signal = ‘AGGT’
      new_signal = ‘GATG’
    else:
      signal = ‘AGG’
      new_signal = ‘GGA’
    while(True):
      if signal in sequence:
        sequence = sequence.replace(signal,new_signal) else:
      return sequence
def filter_enzyme_and_splicing(sequence,which_prime):
  tag = 0
  while(True):
    if has_enzyme(sequence) or has_same_substr_within(sequence):
      sequence = list(sequence)
      random.shuffle(sequence)
      sequence = “”.join(sequence)
    sequence = replace_splicing_signal(sequence,which_prime)
    if not has_enzyme(sequence):
      return sequence
    tag += 1
  if tag > 100:
    return ‘NULL’
if len(sys.argv) != 2:
  print(“Please give the prime type 5prime or 3prime.\nUsage:\npython
generate_random_seq.py 5prime\npython generate_random_seq.py 3prime”)
  exit()
prime_type = sys.argv[1]
if prime_type not in [“5prime”,“3prime”]:
  print(“Please give the prime type 5prime or 3prime.\nUsage:\npython
generate_random_seq.py 5prime\npython generate_random_seq.py 3prime”)
exit()
if prime_type == “5prime”:
num = 50000 # generate 50000 random 5′-fragments for each GC content
else:
  num = 20000 # generate 20000 random 3′-fragments for each GC content
f = open(“{}_random_fragments.txt”.format(prime_type), “w”)
count = 0
for gc_ratio in np.arange(0.55,0.65,0.01):
  for i in range(num):
    count += 1
    while(True):
      seq = generate_sequence(20,gc_ratio,prime_type)
      if seq != ‘NULL’:
        break
    f.write(“>five_short{}\n”.format(count))
    f.write(“{}\n”.format(seq))
```

The 5′-fragment library was used to build the 5′-exonic barcode library, and the 3′-fragment library was used to build the 3′-exonic barcode library. The programming parameters include (1) Each fragment has 20 nucleotides; (2) There are no repeated subfragments longer than 6 nucleotides in each fragment; (3) The GC content ranges from 55% to 65% in each fragment; (4) The fragment does not contain “TTAATTAA”, “GCTAGC”, and their one-miss-match counterparts. “TTAATTAA” and “GCTAGC” are two restriction sites used in AAV vector cloning. “TTAATTAA” is for PacI and “GCTAGC” is for NheI; (5) The fragment does not contain four identical nucleotides in a row, including “AAAA”, “GGGG”, “TTTT”, and “CCCC”; (6) The 5′-fragment library does not contain “Ggt” which is the conserved splicing donor signal (the capital letter is the conserved nucleotide in the exon and the small letters are the conserved nucleotides in the intron) (FIG. 4); (7) The 3′-fragment library does not contain “agG” which is the conserved splicing acceptor signal (the capital letter is the conserved nucleotide in the exon and the small letters are the conserved nucleotides in the intron) (FIG. 4).

In total, the 5′-fragment library contains 500,000 DNA fragments, and the 3′-fragment library contains 200,000 DNA fragments.

### Refinement of the DNA Fragment Libraries

Next, DNA fragments were removed that share high homology with the genome. Since the exonic barcode system was originally planned to be used in human and canine muscles, the random DNA fragment libraries were filtered with pairwise sequence alignment to reduce their sequence identity with the human genome (Homo sapiens, GRCh38) and the dog genome (Canis lupus familiaris, GCF_000002285.3_CanFam3.1). The sequence alignment was performed with the software BLAST 2.9.0 using the following commands. Specifically:
“-task” was set to “blastn”,
“-evalue” (expect value E) was set to 1000,
“-word_size” was set to 7, and
“-max_target_seqs” was set to 5000.

Below is an example of the alignment result:
five_short1, NT_187380.1, 100.000, 13, 0, 0, 4,16,162222, 162210, 613, 24.7 five_short1 (fragment name), NT_187380.1 (genome sequence), 100.000 (identity), 13 (aligned sequence length), 0 (#mismatch), 0 (#gap), 4 (starting index in fragment), 16 (ending index in fragment), 162222 (starting index in genome), 162210 (ending index in genome), 613 (expect value E), 24.7 (bits score)

The BLAST alignment results were analyzed based on the aligned identical sequence length between a query fragment sequence and an object genome sequence (L). L is calculated as the product of the aligned sequence length and the identity (in percentage).

L=(the aligned sequence length)×(the identity)÷100

For a query fragment sequence, there are many Ls corresponding to different aligned regions in the same genome sequence or regions in different genome sequences. Hence, the maximum aligned identical sequence length (maxL) was used to filter the DNA fragment libraries. Specifically, the fragments with a maxL greater than 16 were removed to make the filtered fragments as dissimilar to the genomes as possible.

After refinement, the 5′-fragment library contained 96,223 DNA fragments and the 3′-fragment library contained 137,070 DNA fragments.

### Generation of the Exonic Barcode Libraries

The length of the 5′-exonic barcode was set to 150 nucleotides. To generate the 5′-exonic barcode library, eight fragments were randomly combined from the filtered 5′-fragment library and then the last 10 nucleotides were removed.

The length of the 3′-exonic barcode was set to 50 nucleotides. To generate the 3′-exonic barcode library, three fragments were randomly combined from the filtered 3′-fragment library and then the last 10 nucleotides were removed.

The exonic barcode libraries were further refined with the following parameters including (1) There are no repeated fragments longer than 6 nucleotides in each exonic barcode. In other words, the maximum length of repeated fragments within a single barcode cannot be equal to or longer than 6 nucleotides; (2) The 5′-barcodes must end with “CAG” (the conserved exonic splicing donor signal) (FIG. 4); (3) The 3′-barcodes must start with “G” (the conserved exonic splicing acceptor signal) (FIG. 4); (4) The barcode cannot contain “TTAATTAA”, “GCTAGC”, and their one-miss-match counterparts. “TTAATTAA” and “GCTAGC” are two restriction sites used in AAV vector cloning. “TTAATTAA” is for PacI and “GCTAGC” is for NheI; (5) The barcode cannot contain four identical nucleotides in a row, including “AAAA”, “GGGG”, “TTTT”, and “CCCC”; (6) The 5′-barcode library cannot contain “Ggt” which is the conserved splicing donor signal (the capital letter is the conserved nucleotide in the exon and the small letters are the conserved nucleotides in the intron) (FIG. 4); (7) The 3′-barcode library cannot contain “agG” which is the conserved splicing acceptor signal (the capital letter is the conserved nucleotide in the exon and the small letters are the conserved nucleotides in the intron) (FIG. 4); and (8) The GC content of the barcode is ˜ 60%.

In total, 500,000 5′-barcodes and 500,000 3′-barcodes were generated.

### Refinement of the Barcode Libraries

To reduce the homology of the exonic barcodes with the human genome (Homo sapiens, GRCh38) and the dog genome (Canis lupus familiaris, GCF_000002285.3_CanFam3.1), the barcode libraries were filtered with pairwise sequence alignment using the software BLAST 2.9.0 as was done in the refinement of the DNA fragment libraries.

For the 5′-barcode libraries, the barcodes with a maximum aligned identical sequence length (maxL) greater than 21 were removed. This means there were no identical sequence fragments of lengths greater than 21 nucleotides between the filtered 5′-barcodes and the human/dog genomes. For the 3′-barcode libraries, the barcodes with a maxL greater than 18 were removed. This means there were no identical sequence fragments of lengths greater than 18 nucleotides between the filtered 3′-barcodes and the human/dog genomes.

The candidate barcodes were further refined by removing the ones that contained repeated fragments (≤8 nucleotides) between different barcodes. In other words, the maximum length of repeated fragments among barcodes (5′-barcodes versus 5′-barcodes, 3′-barcodes versus 3′-barcodes, and 5′-barcodes versus 3′-barcodes) cannot be equal to or longer than 8 nucleotides.

In the end, 15 5′-exonic barcodes and 15 3′-exonic barcodes were obtained. The sequences of the 15 5′-exonic barcodes are shown in Table 1, and the sequences of the 15 3′-exonic barcodes are shown in Table 2.

TABLE 1
Sequences of the 15 Candidate 5′-Exonic Barcodes

| 5′- | | |
|---|---|---|
| Exonic | | SEQ |
| Barcode | | ID |
| Number | Sequence | NO |
| 1 | CCGCGTACCCGTGATGACTATCGCGCCGTTATAC | 1 |
| | CACGCAACGGCCATGCTACGACGTATAGTTCGC | |
| | ACGCGATACTCGAGGCGTTGCGCCATTGACGTTT | |
| | CGCGTGGCGCTATTAGTCCGATCCGCGACGACT | |
| | AGTAGCGTAGAGACAG | |
| 2 | TCGTCGCTACGAACGCAACGTAGCGCGACATAC | 2 |
| | CGGCAATGCCCGTAAACGGCATCGTATAGCGAA | |
| | TCCGATCAGTCGTCCTGTTACCGACGCGCAATAC | |
| | TACCCGCGTCACTATACGCTTTAGACGCCTCGCC | |
| | GTTACTTTATTCGCAG | |
| 3 | CGCAAAGCCGAAGTTACGCGGATTGTCGACCCG | 3 |
| | CGGCTTTCGGACATTTCGCGCCGACTATCGTTCG | |
| | GCGCTCGTTATTCGTAGGCGTAATGCCGAGTTGC | |
| | GAACGACGCAAGTACGCCTAACGCCCGTCTACC | |
| | GTACGTGTCGCCGCAG | |
| 4 | CAGCGGAACGCGTACAGTAGCCGTATGCGCGTC | 4 |
| | GCTTAGACGTTTGGCGAACGAACTCGAGTAACA | |
| | CGTTCGCGTTGACCGATTCGTGGCGCATCGCCTA | |
| | ATAATGCGTAGTCGGCGGCGAGTTGTCGACGCG | |
| | CCCAATATCTATGACAG | |
| 5 | CACGGACCACTAATCGGGACCGCAGACGAACCC | 5 |
| | GTTCGAACAAGCGTCGTCGGAGTAACCCACGCG | |
| | AATTCGATGGCCGAACGTTGACGACGCTCGACA | |
| | TTACGCTGCGCGACGTATTGTGCGTAGCGTAAGT | |
| | CGTTTCGTACACGGCAG | |
| 6 | CGCGTACTTCCGACTAACCGTTCCGTAACATACG | 6 |
| | CCCGAGCGGCGCACTACGATATAGACTGGCGCG | |
| | ATCGTCCATCGATGTAGCGCGTGGATGCATCGTT | |
| | TAGCTCGACACCGGCGTGTGTCGAACGTCGCAT | |
| | AACGGACCCGTTGCAG | |
| 7 | TCTAGTGCGACGCGAACGTTTGCCGTACCGTAG | 7 |
| | ACGAGACCCGTTCTACGATCGCCTATCGATCCG | |
| | GCATACCGAGAGTCCTCGTCGCAGTACGCACTTT | |
| | CTCGGCGCGATTGTAGCGTTGTAATCGCGTGCG | |
| | GGCGAATAGTGGCGCAG | |
| 8 | CTGTTCGTACCACACGTCGAGTCCGCGTGATACG | 8 |
| | TTTCGACGATCTATACGCGCGCCACTTGGACGCG | |
| | TTTAACGCCCACCGAGTACGATTACGCCGGACTT | |
| | CGCGATATGCGGACATCGAAGCGTGCGTCCGTA | |
| | TCGAGCATAAAGCAG | |
| 9 | CGCTGATACGACGGATACCGACCATTACTCCGC | 9 |
| | GAGGCGTCGCCCGATTAGTGCATACGGCGACCC | |
| | GCCGACATCGTTAAGACGCAAATTCGCGCTACG | |
| | GGATGAGCGACAGCGTTGCGAAGTACGTCCGGA | |
| | GTCGTAGATAACGGCCAG | |
| 10 | CAACGCCGCGTATGCCTTAAATCCCGCTTACCGC | 10 |
| | ATCGAGATGCGTCGACGGCTGAGTACGCTATAC | |
| | GACCTACGCGACATCGCGTGTAGGCGAAACAAC | |
| | CGTATAACGAAGCGCGGCTAAGATTCGCATGAC | |
| | CGGCCGAACCTGATCAG | |
| 11 | GCGGCGAATTGCAAACGTCGTCCTCGGGCGTAA | 11 |
| | TACACGATACGTCCCGAACGAGACCGTGCTACT | |
| | TAGGCGCGTAGCGAGAACGCGTGTACCGAGGAT | |
| | GCGATTAGATCGATCCACGCGCTGACGCCGTCG | |
| | ATAGTCGTATGCGTCCAG | |
| 12 | ACACGCGTGGAGCGCGAATTGTGATGCGGACGC | 12 |
| | TCGTATCCGCGGAAACGTTCGATAGGGAGTCGT | |
| | GAGCGTGCGACGTAAGCGATGTGCGTTATGCCG | |
| | TATTCCGTGCCCGAATAGGAGGCGCACGATTTG | |
| | TCGTACGCTGCTGCGCAG | |
| 13 | TTGACGGACGCTGTCGCACTAAACGTCGCGACG | 13 |
| | TTACTCCGAACTAATCCGCACCCGCGATGATCGC | |
| | GCTCCAATTCCGTTAATACGTCCACCGGCGCGA | |
| | GACGATAGTACGAGTCGGCTTGATTGCGCGCCG | |
| | CCAATACCATTCGACAG | |
| 14 | GGGCCCGCGACTTATATCGTGACCGTCGTACTAC | 14 |
| | TCCCGTCCGCTGATCACCGCCGTAATCATCGAAC | |
| | GATCGAGTTGGCTCGTAGTCCAATCGACCCGAA | |
| | GTTGTCGCCGAATTGCGAGTCGTTCTATCGGACC | |
| | GGATCTGTATCACAG | |
| 15 | ACAATCGCGGCGTCACGTTAAGCGCTATTTCCG | 15 |
| | GATCGGGCCGAATGTTCCGTACCGACGACCGAT | |
| | GCACGTGCGATATGAGCGCACGGACGTACGAGT | |
| | TTCTACCGCGCGAAAGCGTAAGATGTACGCGTC | |
| | GTAACGCTTACTAGTCAG | |

TBALE 2
Sequences of the 15 Candidate 3′-Exonic Barcodes

| 3′- | | |
|---|---|---|
| Exonic | | SEQ |
| Barcode | | ID |
| Number | Sequence | NO |
| 1 | GGAGCGGACCGTATGTCGACGTCGTTAACGACTCG | 16 |
| | CCGTACGGACATACG | |
| 2 | GGTTATAGCGCGCGTTGTTCCGATTCGCCTCGCGT | 17 |
| | ACGTTACTGGCGGAT | |
| 3 | GGCGGCATTGTCCGCGTAACTCGGTCGCGGATATG | 18 |
| | GTGTGCGCACGACGT | |
| 4 | GGACCGCTATTCGCGACCATATCTCGCGCTTAACGC | 19 |
| | GCGTCCATAGTTGC | |
| 5 | GGACTCGTCTACCAATGCGCGGTCGCACGAATATA | 20 |
| | ACGCGACCGGACAGC | |
| 6 | GGCGCTACACGGAACGCTCATCGAATCGCCGGCCG | 21 |
| | ATAACGTTCCTATTG | |
| 7 | GGCGTCATTACGGCACCGTACTTCGGACGCGGACA | 22 |
| | ATTCGAATAGTCGGC | |
| 8 | GGAGCCGGTTCGGATCGCATATCGCTAATCGCGGA | 23 |
| | GCACGTAGTCGCGAT | |
| 9 | GGAAGCAGCGCGGTTGTAACGACGCGACGGTCCGA | 24 |
| | ATATAGATCGCACGG | |
| 10 | GGCTGATATACACGGCGCACGTCGCGTTATACGGC | 25 |
| | CGGATATCGGAACAC | |
| 11 | GGCCGGATCCGTCGCAATACGATGACTGGCCGTCT | 26 |
| | ATAGCGTGTACGGCG | |
| 12 | GGATCGCGACCTAACCTCGATCGAAGACCGCACGT | 27 |
| | AACGGTATAGTCCGG | |
| 13 | GGAGCACTTGCGTACTCGACCGGTATACGCCATAA | 28 |
| | CGGTCTATCACGCCT | |
| 14 | GGATTCCGGACGTCGTACGTCTATCCGCCGAATGAC | 29 |
| | GGTCGAGCGACCTT | |
| 15 | GGTACAATCCACTCGATCCGACGGCGGATGCAACG | 30 |
| | TACGTGACGAAGTGC | |

Next, the 30 exonic barcodes were analyzed with the software BLAST 2.9.0 to confirm that these barcodes indeed have low sequence identity with the human and dog genomes. The Blast search results of the 5′-exonic barcodes and the Blast search results of the 3′-exonic barcodes were conducted separately. The Blast search summary is shown in Table 3.

TABLE 3
Blast Evaluation of Candidate Exonic Barcodes in the Human and Dog Genome

| | Human | | Dog | | | Human | | Dog | |
|---|---|---|---|---|---|---|---|---|---|
| | genome | | genome | | | genome | | genome | |
| | | E | | E | | | E | | E |
| | maxL | value | maxL | value | | maxL | value | maxL | value |
| 5′-barcode 1 | 18 | 571 | 18 | 571 | 3′-barcode 1 | 17 | 741 | 16 | 101 |
| 5′-barcode 2 | 19 | 571 | 19 | 571 | 3′-barcode 2 | 15 | 352 | 17 | 352 |
| 5′-barcode 3 | 19 | 571 | 19 | 571 | 3′-barcode 3 | 17 | 352 | 17 | 29 |
| 5′-barcode 4 | 19 | 571 | 19 | 571 | 3′-barcode 4 | 17 | 352 | 17 | 352 |
| 5′-barcode 5 | 19 | 571 | 19 | 57 | 3′-barcode 5 | 16 | 101 | 17 | 352 |
| 5′-barcode 6 | 19 | 164 | 19 | 571 | 3′-barcode 6 | 18 | 352 | 18 | 352 |
| 5′-barcode 7 | 19 | 571 | 19 | 57 | 3′-barcode 7 | 18 | 352 | 18 | 352 |
| 5′-barcode 8 | 20 | 164 | 19 | 571 | 3′-barcode 8 | 18 | 352 | 18 | 352 |
| 5′-barcode 9 | 20 | 47 | 20 | 164 | 3′-barcode 9 | 18 | 352 | 18 | 352 |
| 5′-barcode 10 | 21 | 47 | 21 | 571 | 3′-barcode 10 | 15 | 352 | 18 | 352 |
| 5′-barcode 11 | 21 | 571 | 21 | 571 | 3′-barcode 11 | 18 | 352 | 18 | 352 |
| 5′-barcode 12 | 21 | 13 | 21 | 13 | 3′-barcode 12 | 18 | 352 | 17 | 352 |
| 5′-barcode 13 | 21 | 47 | 21 | 47 | 3′-barcode 13 | 18 | 101 | 18 | 352 |
| 5′-barcode 14 | 21 | 571 | 21 | 57 | 3′-barcode 14 | 18 | 352 | 18 | 352 |
| 5′-barcode 15 | 21 | 47 | 18 | 571 | 3′-barcode 15 | 18 | 352 | 18 | 352 |

In the human genome, (i) the maxL values of the 5′- and 3′-exonic barcodes are 18 to 21 and 15 to 18, respectively, suggesting minimum homology; (ii) the E-values of the 5′- and 3′-exonic barcodes are 13 to 571 and 101 to 741, respectively, suggesting they are not good hits for homology matches. In the dog genome, (i) the maxL values of the 5′- and 3′-exonic barcodes are 18 to 21 and 16 to 18, respectively, suggesting minimum homology; (ii) the E-values of the 5′- and 3′-exonic barcodes are 13 to 571 and 101 to 352, respectively, suggesting they are not good hits for homology matches.

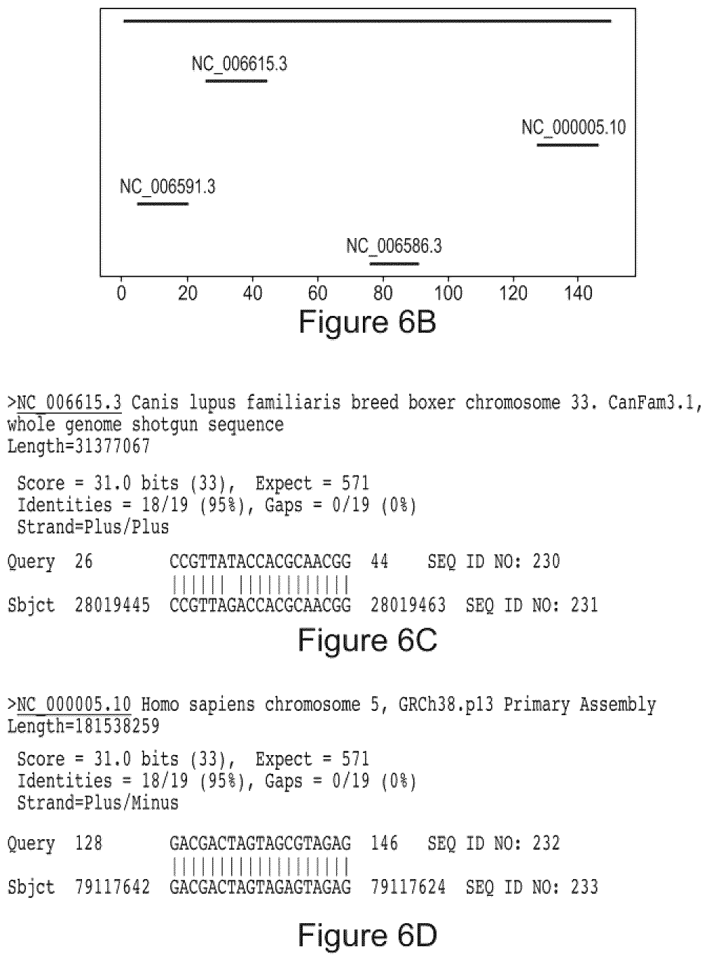

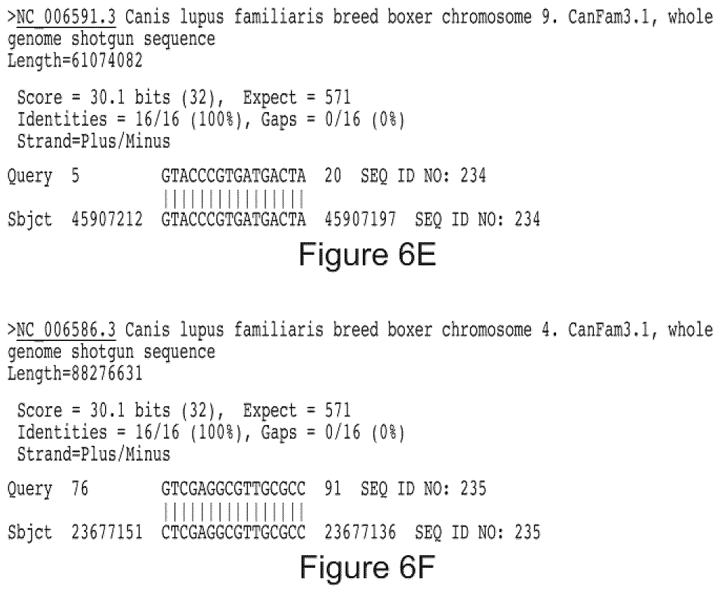

FIG. 6A-6F shows a representative Blast search result (5′-exonic barcode 1). Four regions of this barcode share homology with the human/dog genome sequence. For example, result NC_006615.3 shows that the nucleotides 26 to 44 of 5′-exonic barcode 1 share homology with a region (from 28019445 to 28019463) in chromosome 33 of the dog genome (28019445 is the starting index in the dog genome and 28019463 is the ending index in the dog genome) (FIG. 6C). The bits score is 31.0, and the E value is 571. The identity is 18/19 (95%), indicating one mismatch between the barcode sequence and the genome sequence.

Examination of 30 Refined Exonic Barcodes for the Sequence Identity with the Genomes of Other Five Commonly Used Mammalian Experimental Models

During the bioinformatic design of the exonic barcodes, sequence identity with the human genome and the dog genome was considered (Table 3). To expand the utility of the exonic barcodes in preclinical studies, the sequence similarities were examined between the finalized exonic barcodes and the genomes of the other five species, including rat (Rattus norvegicus, GCF_015227675.2_mRatBN7.2), mouse (Mus musculus, GCF_000001635.27_GRCm39), monkey (Macaca mulatta, GCF_003339765.1_Mmul_10), pig (Sus scrofa, GCF_000003025.6_Sscrofa11.1), and rabbit (Oryctolagus cuniculus, GCF_000003625.3_OryCun2.0). The Blast search results of the exonic barcodes with these genomes were conducted separately. Overall, bioinformatic analysis suggests that the customer-designed exonic barcodes share minimum homology to the genomic sequences in rats, mice, monkeys, pigs, and rabbits. Hence, this barcode system can also be used in these 5 species.

The Blast search results for all 7 species are summarized in Tables 4 and 5.

TABLE 4
Blast Search of the 5′-Exonic Barcodes in Genomes of 7 Species

| | Rats | Mice | Monkeys | Pigs | Rabbits | Humans | Dogs |
|---|---|---|---|---|---|---|---|
| 5′-barcode 1 | 23/937 | 23/79 | 29/301 | 27/254 | 27/23 | 18/571 | 18/571 |
| 5′-barcode 2 | 24/937 | 30/966 | 23/86 | 24/254 | 26/969 | 19/571 | 19/571 |
| 5′-barcode 3 | 25/937 | 24/23 | 22/301 | 26/73 | 35/80 | 19/571 | 19/571 |
| 5′-barcode 4 | 27/937 | 25/79 | 35/301 | 32/254 | 33/969 | 19/571 | 19/571 |
| 5′-barcode 5 | 25/77 | 25/277 | 27/301 | 25/254 | 30/969 | 19/571 | 19/571 |
| 5′-barcode 6 | 25/937 | 25/277 | 25/7.1 | 27/254 | 29/23 | 19/164 | 19/571 |
| 5′-barcode 7 | 28/269 | 27/277 | 25/86 | 31/1.7 | 33/80 | 19/571 | 19/571 |
| 5′-barcode 8 | 28/269 | 25/966 | 28/301 | 28/73 | 28/278 | 20/164 | 19/571 |
| 5′-barcode 9 | 24/269 | 31/966 | 22/301 | 31/0.49 | 32/0.04 | 20/47 | 20/164 |
| 5′-barcode 10 | 30/269 | 26/966 | 24/301 | 28/886 | 24/278 | 21/47 | 21/571 |
| 5′-barcode 11 | 31/269 | 32/79 | 33/301 | 27/73 | 33/278 | 21/571 | 21/571 |
| 5′-barcode 12 | 25/937 | 29/966 | 28/2.0 | 29/886 | 28/278 | 21/13 | 21/13 |
| 5′-barcode 13 | 24/937 | 25/277 | 24/301 | 24/254 | 29/80 | 21/47 | 21/47 |
| 5′-barcode 14 | 27/269 | 38/966 | 22/86 | 27/254 | 29/969 | 21/571 | 21/571 |
| 5′-barcode 15 | 26/22 | 27/277 | 22/301 | 27/254 | 35/80 | 21/47 | 18/571 |

*The value before the slash is maxL and the value after the slash is the E-value.

TABLE 5
Blast Search of the 3′-Exonic Barcodes in Genomes of 7 Species

| | Rats | Mice | Monkeys | Pigs | Rabbits | Humans | Dogs |
|---|---|---|---|---|---|---|---|
| 3′-barcode 1 | 23/600 | 20/618 | 17/673 | 26/567 | 20/178 | 17/741 | 16/101 |
| 3′-barcode 2 | 23/600 | 23/15 | 22/16 | 23/13 | 20/178 | 15/352 | 17/352 |
| 3′-barcode 3 | 22/600 | 28/177 | 26/673 | 26/567 | 21/178 | 17/352 | 17/29 |
| 3′-barcode 4 | 26/600 | 21/177 | 25/673 | 20/162 | 23/620 | 17/352 | 17/352 |
| 3′-barcode 5 | 24/49 | 22/618 | 22/673 | 23/162 | 26/620 | 16/101 | 17/352 |
| 3′-barcode 6 | 26/14 | 23/618 | 20/673 | 23/567 | 27/4.2 | 18/352 | 18/352 |
| 3′-barcode 7 | 20/600 | 21/177 | 20/673 | 23/567 | 26/620 | 18/352 | 18/352 |
| 3′-barcode 8 | 23/600 | 22/618 | 22/673 | 22/567 | 21/178 | 18/352 | 18/352 |
| 3′-barcode 9 | 24/172 | 19/618 | 22/673 | 23/567 | 24/178 | 18/352 | 18/352 |
| 3′-barcode 10 | 20/172 | 19/618 | 23/673 | 19/47 | 21/51 | 15/352 | 18/352 |
| 3′-barcode 11 | 28/49 | 25/177 | 27/55 | 26/162 | 26/620 | 18/352 | 18/352 |
| 3′-barcode 12 | 23/172 | 23/177 | 19/673 | 24/162 | 28/620 | 18/352 | 17/352 |
| 3′-barcode 13 | 23/600 | 21/177 | 23/193 | 23/162 | 22/178 | 18/101 | 18/352 |
| 3′-barcode 14 | 23/49 | 21/177 | 20/673 | 22/567 | 21/178 | 18/352 | 18/352 |
| 3′-barcode 15 | 26/172 | 23/618 | 20/673 | 23/567 | 26/15 | 18/352 | 18/352 |

The value before the slash is maxL and the value after the slash is the E-value.

### Evaluation of Alternative Splice Sites in 15 Pairs of Refined Exonic Barcodes

To further refine the exonic barcodes, potential alternative splice sites in the intact barcodes were examined with the alternative splice site predictor software (ASSP) (Wang & Marin, 2006). The intact barcode was generated by joining the sequence of 5′-exonic barcode with the sequence of the synthetic intron and the sequence of the corresponding 3′-exonic barcode in the order of: (from 5′ to 3′) 5′-exonic barcode, synthetic intron, and 3′-exonic barcode. The sequences of the 15 intact barcodes are shown in Table 6. Capital letters indicate exonic sequence, and small letters indicate intronic sequence.

TABLE 6
Exonic Barcodes

| Exonic | | SEQ |
|---|---|---|
| Barcode | | ID |
| Number | Sequence | NO |
| 1 | CCGCGTACCCGTGATGACTATCGCGCCGTTATACC | 31 |
| | ACGCAACGGCCATGCTACGACGTATAGTTCGCACG | |
| | CGATACTCGAGGCGTTGCGCCATTGACGTTTCGCG | |
| | TGGCGCTATTAGTCCGATCCGCGACGACTAGTAGC | |
| | GTAGAGACAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGAGCGGACCGTATGTCGACGTCGTTAACGAC | |
| | TCGCCGTACGGACATACG | |
| 2 | TCGTCGCTACGAACGCAACGTAGCGCGACATACCG | 32 |
| | GCAATGCCCGTAAACGGCATCGTATAGCGAATCCG | |
| | ATCAGTCGTCCTGTTACCGACGCGCAATACTACCC | |
| | GCGTCACTATACGCTTTAGACGCCTCGCCGTTACT | |
| | TTATTCGCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGTTATAGCGCGCGTTGTTCCGATTCGCCTCG | |
| | CGTACGTTACTGGCGGAT | |
| 3 | CGCAAAGCCGAAGTTACGCGGATTGTCGACCCGCG | 33 |
| | GCTTTCGGACATTTCGCGCCGACTATCGTTCGGCG | |
| | CTCGTTATTCGTAGGCGTAATGCCGAGTTGCGAAC | |
| | GACGCAAGTACGCCTAACGCCCGTCTACCGTACGT | |
| | GTCGCCGCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGCGGCATTGTCCGCGTAACTCGGTCGCGGAT | |
| | ATGGTGTGCGCACGACGT | |
| 4 | CAGCGGAACGCGTACAGTAGCCGTATGCGCGTCGC | 34 |
| | TTAGACGTTTGGCGAACGAACTCGAGTAACACGTT | |
| | CGCGTTGACCGATTCGTGGCGCATCGCCTAATAAT | |
| | GCGTAGTCGGCGGCGAGTTGTCGACGCGCCCAATA | |
| | TCTATGACAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGACCGCTATTCGCGACCATATCTCGCGCTTA | |
| | ACGCGCGTCCATAGTTGC | |
| 5 | CACGGACCACTAATCGGGACCGCAGACGAACCCGT | 35 |
| | TCGAACAAGCGTCGTCGGAGTAACCCACGCGAATT | |
| | CGATGGCCGAACGTTGACGACGCTCGACATTACGC | |
| | TGCGCGACGTATTGTGCGTAGCGTAAGTCGTTTCG | |
| | TACACGGCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGACTCGTCTACCAATGCGCGGTCGCACGAAT | |
| | ATAACGCGACCGGACAGC | |
| 6 | CGCGTACTTCCGACTAACCGTTCCGTAACATACGC | 36 |
| | CCGAGCGGCGCACTACGATATAGACTGGCGCGATC | |
| | GTCCATCGATGTAGCGCGTGGATGCATCGTTTAGC | |
| | TCGACACCGGCGTGTGTCGAACGTCGCATAACGGA | |
| | CCCGTTGCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGCGCTACACGGAACGCTCATCGAATCGCCGG | |
| | CCGATAACGTTCCTATTG | |
| 7 | TCTAGTGCGACGCGAACGTTTGCCGTACCGTAGAC | 37 |
| | GAGACCCGTTCTACGATCGCCTATCGATCCGGCAT | |
| | ACCGAGAGTCCTCGTCGCAGTACGCACTTTCTCGG | |
| | CGCGATTGTAGCGTTGTAATCGCGTGCGGGCGAAT | |
| | AGTGGCGCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGCGTCATTACGGCACCGTACTTCGGACGCGG | |
| | ACAATTCGAATAGTCGGC | |
| 8 | CTGTTCGTACCACACGTCGAGTCCGCGTGATACGT | 38 |
| | TTCGACGATCTATACGCGCGCCACTTGGACGCGTT | |
| | TAACGCCCACCGAGTACGATTACGCCGGACTTCGC | |
| | GATATGCGGACATCGAAGCGTGCGTCCGTATCGAG | |
| | CATAAAGCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGAGCCGGTTCGGATCGCATATCGCTAATCGC | |
| | GGAGCACGTAGTCGCGAT | |
| 9 | CGCTGATACGACGGATACCGACCATTACTCCGCGA | 39 |
| | GGCGTCGCCCGATTAGTGCATACGGCGACCCGCCG | |
| | ACATCGTTAAGACGCAAATTCGCGCTACGGGATGA | |
| | GCGACAGCGTTGCGAAGTACGTCCGGAGTCGTAGA | |
| | TAACGGCCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGAAGCAGCGCGGTTGTAACGACGCGACGGTC | |
| | CGAATATAGATCGCACGG | |
| 10 | CAACGCCGCGTATGCCTTAAATCCCGCTTACCGCA | 40 |
| | TCGAGATGCGTCGACGGCTGAGTACGCTATACGAC | |
| | CTACGCGACATCGCGTGTAGGCGAAACAACCGTAT | |
| | AACGAAGCGCGGCTAAGATTCGCATGACCGGCCGA | |
| | ACCTGATCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGCTGATATACACGGCGCACGTCGCGTTATAC | |
| | GGCCGGATATCGGAACAC | |
| 11 | GCGGCGAATTGCAAACGTCGTCCTCGGGCGTAATA | 41 |
| | CACGATACGTCCCGAACGAGACCGTGCTACTTAGG | |
| | CGCGTAGCGAGAACGCGTGTACCGAGGATGCGATT | |
| | AGATCGATCCACGCGCTGACGCCGTCGATAGTCGT | |
| | ATGCGTCCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGCCGGATCCGTCGCAATACGATGACTGGCCG | |
| | TCTATAGCGTGTACGGCG | |
| 12 | ACACGCGTGGAGCGCGAATTGTGATGCGGACGCTC | 42 |
| | GTATCCGCGGAAACGTTCGATAGGGAGTCGTGAGC | |
| | GTGCGACGTAAGCGATGTGCGTTATGCCGTATTCC | |
| | GTGCCCGAATAGGAGGCGCACGATTTGTCGTACGC | |
| | TGCTGCGCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGATCGCGACCTAACCTCGATCGAAGACCGCA | |
| | CGTAACGGTATAGTCCGG | |
| 13 | TTGACGGACGCTGTCGCACTAAACGTCGCGACGTT | 43 |
| | ACTCCGAACTAATCCGCACCCGCGATGATCGCGCT | |
| | CCAATTCCGTTAATACGTCCACCGGCGCGAGACGA | |
| | TAGTACGAGTCGGCTTGATTGCGCGCCGCCAATAC | |
| | CATTCGACAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGAGCACTTGCGTACTCGACCGGTATACGCCA | |
| | TAACGGTCTATCACGCCT | |
| 14 | GGGCCCGCGACTTATATCGTGACCGTCGTACTACT | 44 |
| | CCCGTCCGCTGATCACCGCCGTAATCATCGAACGA | |
| | TCGAGTTGGCTCGTAGTCCAATCGACCCGAAGTTG | |
| | TCGCCGAATTGCGAGTCGTTCTATCGGACCGGATC | |
| | TGTATCACAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGATTCCGGACGTCGTACGTCTATCCGCCGAA | |
| | TGACGGTCGAGCGACCTT | |
| 15 | ACAATCGCGGCGTCACGTTAAGCGCTATTTCCGGA | 45 |
| | TCGGGCCGAATGTTCCGTACCGACGACCGATGCAC | |
| | GTGCGATATGAGCGCACGGACGTACGAGTTTCTAC | |
| | CGCGCGAAAGCGTAAGATGTACGCGTCGTAACGCT | |
| | TACTAGTCAGgtaagtatcaaggttacaagacagg | |
| | tttaaggagaccaatagaaactgggcttgtcgaga | |
| | cagagaagactcttgcgtttctgataggcacctat | |
| | tggtcttactgacatccactttgcctttctctcca | |
| | cagGGTACAATCCACTCGATCCGACGGCGGATGCA | |
| | ACGTACGTGACGAAGTGC | |

The results of ASSP analysis are shown in Table 7.

TABLE 7
ASSP Analysis of Splice Signals in Exonic Barcodes

| Exonic | | Putative | Sequence | SEQ | Splice |
|---|---|---|---|---|---|
| Barcode | Position | splice | (capital, putative exon) | ID | strength |
| Number | (bp) | signal | (small, putative intron) | NO | score |
| 1 | 150 | Donor | GTAGAGACAGgtaagtatca | 46 | 13.642 |
| | 162 | Donor | AAGTATCAAGgttacaagac | 47 | 6.543 |
| | 174 | Donor | TACAAGACAGgtttaaggag | 48 | 4.665 |
| | 238 | Acceptor | tttctgatagGCACCTATTG | 49 | 6.257 |
| | 284 | Acceptor | ctctccacagGGAGCGGACC | 50 | 12.832 |
| 2 | 125 | Acceptor | tacgctttagACGCCTCG CC | 51 | 2.443 |
| | 150 | Donor | TTATTCGCAGgtaagtatca | 52 | 14.223 |
| | 151 | Acceptor | ttattcgcagGTAAGTATCA | 53 | 10.718* |
| | 162 | Donor | AAGTATCAAGgttacaagac | 54 | 6.543 |
| | 174 | Donor | TACAAGACAGgtttaaggag | 55 | 4.665 |
| | 238 | Acceptor | tttctgatagGCACCTATTG | 56 | 6.257 |
| | 284 | Acceptor | ctctccacagGGTTATAGCG | 57 | 12.832 |
| 3 | 85 | Acceptor | ttattcgtagGCGTAATG CC | 58 | 6.375 |
| | 134 | Donor | CCCGTCTACCgtacgtgtcg | 59 | 6.53 |
| | 150 | Donor | GTCGCCGCAGgtaagtatca | 60 | 13.235 |
| | 151 | Acceptor | gtcgccgcagGTAAGTATCA | 61 | 6.029 |
| | 162 | Donor | AAGTATCAAGgttacaagac | 62 | 6.543 |
| | 174 | Donor | TACAAGACAGgtttaaggag | 63 | 4.665 |
| | 238 | Acceptor | tttctgatagGCACCTATTG | 64 | 6.257 |
| | 284 | Acceptor | ctctccacagGGCGGCATTG | 65 | 12.832 |
| 4 | 60 | Donor | ACGAACTCGAgtaacacgtt | 66 | 5.142 |
| | 150 | Donor | TCTATGACAGgtaagtatca | 67 | 14.625 |
| | 151 | Acceptor | tctatgacagGTAAGTATCA | 68 | 3.32 |
| | 162 | Donor | AAGTATCAAGgttacaagac | 69 | 6.543 |
| | 174 | Donor | TACAAGACAGgtttaaggag | 70 | 4.665 |
| | 238 | Acceptor | tttctgatagGCACCTATTG | 71 | 6.257 |
| | 284 | Acceptor | ctctccacagGGACCGCTAT | 72 | 12.832 |
| 5 | 118 | Donor | GCGACGTATTgtgcgtagcg | 73 | 5.519 |
| | 127 | Donor | TGTGCGTAGCgtaagtcgtt | 74 | 8.701 |
| | 150 | Donor | TACACGGCAGgtaagtatca | 75 | 13.144 |
| | 151 | Acceptor | tacacggcagGTAAGTATCA | 76 | 5.586 |
| | 162 | Donor | AAGTATCAAGgttacaagac | 77 | 6.543 |
| | 174 | Donor | TACAAGACAGgtttaaggag | 78 | 4.665 |
| | 238 | Acceptor | tttctgatagGCACCTATTG | 79 | 6.257 |
| | 284 | Acceptor | ctctccacagGGACTCGTCT | 80 | 12.832 |
| 6 | 150 | Donor | CCCGTTGCAGgtaagtatca | 81 | 13.731 |
| | 151 | Acceptor | cccgttgcagGTAAGTATCA | 82 | 3.593 |
| | 162 | Donor | AAGTATCAAGgttacaagac | 83 | 6.543 |
| | 174 | Donor | TACAAGACAGgtttaaggag | 84 | 4.665 |
| | 238 | Acceptor | tttctgatagGCACCTATTG | 85 | 6.257 |
| | 284 | Acceptor | ctctccacagGGCGCTACAC | 86 | 12.832 |
| 7 | 89 | Donor | CCTCGTCGCAgtacgcactt | 87 | 4.79 |
| | 91 | Acceptor | ctcgtcgcagTACGCACTTT | 88 | 3.191 |
| | 150 | Donor | AGTGGCGCAGgtaagtatca | 89 | 13.577 |
| | 162 | Donor | AAGTATCAAGgttacaagac | 90 | 6.543 |
| | 174 | Donor | TACAAGACAGgtttaaggag | 91 | 4.665 |
| | 238 | Acceptor | tttctgatagGCACCTATTG | 92 | 6.257 |
| | 284 | Acceptor | ctctccacagGGCGTCATTA | 93 | 12.832 |
| 8 | 150 | Donor | CATAAAGCAGgtaagtatca | 94 | 14.112 |
| | 162 | Donor | AAGTATCAAGgttacaagac | 95 | 6.543 |
| | 174 | Donor | TACAAGACAGgtttaaggag | 96 | 4.665 |
| | 238 | Acceptor | tttctgatagGCACCTATTG | 97 | 6.257 |
| | 284 | Acceptor | ctctccacagGGAGCCGGTT | 98 | 12.832 |
| 9 | 121 | Donor | GCGTTGCGAAgtacgtccgg | 99 | 6.72 |
| | 150 | Donor | TAACGGCCAGgtaagtatca | 100 | 13.501 |
| | 162 | Donor | AAGTATCAAGgttacaagac | 101 | 6.543 |
| | 174 | Donor | TACAAGACAGgtttaaggag | 102 | 4.665 |
| | 238 | Acceptor | tttctgatagGCACCTATTG | 103 | 6.257 |
| | 284 | Acceptor | ctctccacagGGAAGCAGCG | 104 | 12.832 |
| 10 | 150 | Donor | ACCTGATCAGgtaagtatca | 105 | 14.079 |
| | 154 | Donor | GATCAGGTAAgtatcaaggt | 106 | 5 |
| | 162 | Donor | AAGTATCAAGgttacaagac | 107 | 6.543 |
| | 174 | Donor | TACAAGACAGgtttaaggag | 108 | 4.665 |
| | 238 | Acceptor | tttctgatagGCACCTATTG | 109 | 6.257 |
| | 284 | Acceptor | ctctccacagGGCTGATATA | 110 | 12.832 |
| 11 | 150 | Donor | ATGCGTCCAGgtaagtatca | 111 | 14.02 |
| | 151 | Acceptor | atgcgtccagGTAAGTATCA | 112 | 4.924 |
| | 162 | Donor | AAGTATCAAGgttacaagac | 113 | 6.543 |
| | 174 | Donor | TACAAGACAGgtttaaggag | 114 | 4.665 |
| | 238 | Acceptor | tttctgatagGCACCTATTG | 115 | 6.257 |
| | 284 | Acceptor | ctctccacagGGCCGGATCC | 116 | 12.832 |
| 12 | 77 | Donor | AGCGTGCGACgtaagcgatg | 117 | 6.866 |
| | 86 | Donor | CGTAAGCGATgtgcgttatg | 118 | 6.275 |
| | 118 | Acceptor | gcccgaatagGAGGCGCACG | 119 | 2.695 |
| | 150 | Donor | TGCTGCGCAGgtaagtatca | 120 | 13.443 |
| | 151 | Acceptor | tgctgcgcagGTAAGTATCA | 121 | 5.009 |
| | 162 | Donor | AAGTATCAAGgttacaagac | 122 | 6.543 |
| | 174 | Donor | TACAAGACAGgtttaaggag | 123 | 4.665 |
| | 238 | Acceptor | tttctgatagGCACCTATTG | 124 | 6.257 |
| | 284 | Acceptor | ctctccacagGGATCGCGAC | 125 | 12.832 |
| 13 | 150 | Donor | CATTCGACAGgtaagtatca | 126 | 14.884 |
| | 151 | Acceptor | cattcgacagGTAAGTATCA | 127 | 2.884 |
| | 162 | Donor | AAGTATCAAGgttacaagac | 128 | 6.543 |
| | 174 | Donor | TACAAGACAGgtttaaggag | 129 | 4.665 |
| | 238 | Acceptor | tttctgatagGCACCTATTG | 130 | 6.257 |
| | 284 | Acceptor | ctctccacagGGAGCACTTG | 131 | 12.832 |
| 14 | 150 | Donor | TGTATCACAGgtaagtatca | 132 | 14.006 |
| | 151 | Acceptor | tgtatcacagGTAAGTATCA | 133 | 5.19 |
| | 162 | Donor | AAGTATCAAGgttacaagac | 134 | 6.543 |
| | 174 | Donor | TACAAGACAGgtttaaggag | 135 | 4.665 |
| | 238 | Acceptor | tttctgatagGCACCTATTG | 136 | 6.257 |
| | 284 | Acceptor | ctctccacagGGATTCCGGA | 137 | 12.832 |
| 15 | 116 | Donor | GCGCGAAAGCgtaagatgta | 138 | 5.703 |
| | 123 | Donor | AGCGTAAGATgtacgcgtcg | 139 | 4.867 |
| | 150 | Donor | TACTAGTCAGgtaagtatca | 140 | 13.884 |
| | 151 | Acceptor | tactagtcagGTAAGTATCA | 141 | 3.535 |
| | 162 | Donor | AAGTATCAAGgttacaagac | 142 | 6.543 |
| | 174 | Donor | TACAAGACAGgtttaaggag | 143 | 4.665 |
| | 238 | Acceptor | tttctgatagGCACCTATTG | 144 | 6.257 |
| | 284 | Acceptor | ctctccacagGGTACAATCC | 145 | 12.832 |

In 14 exonic barcodes, the splice strength of the expected splice donor and accepter had the highest score in each respective barcode (all >10). However, in barcode #2, two accepter signals were found with a splice strength higher than 10, indicating potential multiple splicing events. For this reason, this barcode was excluded. In the end, a total of 14 exonic barcodes were obtained. These are the same barcodes reported in Table 6, with exonic barcode #2 excluded (SEQ ID NOs: 31 and 33-45).

The GC content of the exonic barcodes was also calculated. The results are shown in Table 8 below.

TABLE 8
GC content of exonic barcodes

| | 5′- | | | in full length |
|---|---|---|---|---|
| barcode pair # | barcode | intron | 3′-barcode | (5′-intron-3′) |
| 1 | 58.0% | 44.4% | 60.0% | 52.9% |
| 3 | 60.0% | 44.4% | 64.0% | 54.4% |
| 4 | 56.7% | 44.4% | 58.0% | 52.0% |
| 5 | 58.0% | 44.4% | 60.0% | 52.9% |
| 6 | 58.7% | 44.4% | 58.0% | 52.9% |
| 7 | 58.7% | 44.4% | 58.0% | 52.9% |
| 8 | 56.7% | 44.4% | 60.0% | 52.3% |
| 9 | 59.3% | 44.4% | 60.0% | 53.5% |
| 10 | 56.7% | 44.4% | 58.0% | 52.0% |
| 11 | 58.7% | 44.4% | 62.0% | 53.5% |
| 12 | 59.3% | 44.4% | 58.0% | 53.2% |
| 13 | 57.3% | 44.4% | 56.0% | 52.0% |
| 14 | 56.0% | 44.4% | 60.0% | 52.0% |
| 15 | 56.0% | 44.4% | 58.0% | 51.7% |

### Example 3: Evaluation of the 14 Exonic Barcodes by Taqman™ PCR

### Design of Primers and Probes for TagMan™ PCR Quantification of the Vector Genome Copy Number and Transcript Copy Number

To accurately quantify the transduction and expression of barcoded AAV vectors in animals, 28 sets of unique primers and probes were designed. 14 sets were designed to evaluate transduction efficiency by quantifying the vector genome copy number. These primers/probes should generate an ˜60 bp amplicon targeting the 5′-exonic barcodes (FIG. 3A left panel). These primers/probes are listed in Table 9.

TABLE 9
Primers and Probes to Quantify the Vector Genome Copy Number (Vector Transduction)

| Barcode- | 5′-primer | Probe | 3′-primer | Amplicon |
|---|---|---|---|---|
| name | (SEQ ID NO) | (SEQ ID NO) | (SEQ ID NO) | size |
| barcode | GGCCATGCTACGACGTA | TCGCACGCGATA | CCACGCGAAACGTCAATG | 66 bp |
| #1 | TAGT (146) | CTC (160) | G (174) | |
| barcode | GCGGCTTTCGGACATTTC | TCGGCGCTCGTTA | GTTCGCAACTCGGCATTAC | 73 bp |
| #3 | G (147) | TT (161) | G (175) | |
| barcode | GAGTAACACGTTCGCGT | ATGCGCCACGAA | CCGCCGACTACGCATTATT | 60 bp |
| #4 | TGAC (148) | TCG (162) | AGG (176) | |
| barcode | AAGCGTCGTCGGAGTAA | TCGGCCATCGAA | TCGAGCGTCGTCAACGT | 56 bp |
| #5 | CC (149) | TTC (163) | (177) | |
| barcode | CGGCGCACTACGATATA | CCACGCGCTACA | GGTGTCGAGCTAAACGAT | 73 bp |
| #6 | GACT (150) | TCG (164) | GCA (178) | |
| barcode | TCGTCGCAGTACGCACTT | CTCGGCGCGATT | CCCGCACGCGATTACAAC | 54 bp |
| #7 | T (151) | GTAG (165) | (179) | |
| barcode | GCCACTTGGACGCGTTT | ACGCCCACCGAG | CGCGAAGTCCGGCGTAA | 52 bp |
| #8 | (152) | TACG (166) | (180) | |
| barcode | TCGCCCGATTAGTGCAT | ACCCGCCGACAT | GTAGCGCGAATTTGCGTCT | 59 bp |
| #9 | ACG (153) | CG (167) | TAA (181) | |
| barcode | CGCGTGTAGGCGAAACA | CCGCGCTTCGTTA | GCCGGTCATGCGAATCTTA | 56 bp |
| #10 | AC (154) | TAC (168) | G (182) | |
| barcode | CCGAACGAGACCGTGCT | TCTCGCTACGCGC | GATCTAATCGCATCCTCGG | 64 bp |
| #11 | A (155) | CTAA (169) | TACAC (183) | |
| barcode | CGCGGAAACGTTCGATA | ACGTCGCACGCT | GGAATACGGCATAACGCA | 65 bp |
| #12 | GG (156) | CAC (170) | CATC (184) | |
| barcode | GCGACGTTACTCCGAAC | TTGGAGCGCGAT | GCGCCGGTGGACGTATTA | 71 bp |
| #13 | TAATCC (157) | CATC (171) | A (185) | |
| barcode | CTACTCCCGTCCGCTGAT | CG (172) | CCGCCGTAATCATTCGGG | 70 bp |
| #14 | C (158) | | TCGATTGGACTACGA (186) | |
| barcode | GTACCGACGACCGATGC | TCCGTGCGCTCAT | GCGCGGTAGAAACTCGTA | 59 bp |
| #15 | A (159) | ATC (173) | C (187) | |

14 separate sets were designed to evaluate exonic barcode expression (transcript copy number). These primers/probes should generate an ˜60 bp amplicon targeting the junction region between the 5′- and 3′-exonic barcodes (FIG. 3a right panel). These primers/probes are listed in Table 10.

TABLE 10
Primers and Probes to Quantify the Transcript Copy Number (Vector Expression)

| Barcode- | 5′-primer | Probe | 3′-primer | Amplicon |
|---|---|---|---|---|
| name | (SEQ ID NO) | (SEQ ID NO) | (SEQ ID NO) | size |
| barcode | CGATCCGCGACGACTA | TCCGCTCCCTGTCTCTA | CGTTAACGACGTCGACA | 61 bp |
| #1 | GTAG (188) | (202) | TACG (216) | |
| barcode | GCCCGTCTACCGTACG | CCGCCCTGCGGCGAC | ACCGAGTTACGCGGAC | 52 bp |
| #3 | T (189) | (203) | AATG (217) | |
| barcode | TGTCGACGCGCCCAAT | AATAGCGGTCCCTGTC | GCGTTAAGCGCGAGAT | 63 bp |
| #4 | AT (190) | ATAG (204) | ATGGT (218) | |
| barcode | GCGTAGCGTAAGTCGT | ACGGCAGGGACTCGTC | IGCGACCGCGCATTGG | 56 bp |
| #5 | TTCGTA (191) | (205) | (219) | |
| barcode | GTGTCGAACGTCGCAT | TTGCAGGGCGCTACAC | GCCGGCGATTCGATGA | 65 bp |
| #6 | AACG (192) | (206) | G (220) | |
| barcode | GCGTGCGGGCGAAT | ACGCCCTGCGCCACT | TGTCCGCGTCCGAAGTA | 59 bp |
| #7 | (193) | (207) | C (221) | |
| barcode | GCGTGCGTCCGTATCG | CCGGCTCCCTGCTTTA | CTCCGCGATTAGCGATA | 64 bp |
| #8 | A (194) | (208) | TGC (222) | |
| barcode | ACAGCGTTGCGAAGTA | CTTCCCTGGCCGTTATC | CCGTCGCGTCGTTACAA | 72 bp |
| #9 | CGT (195) | (209) | C (223) | |
| barcode | CATGACCGGCCGAACC | CCGTGTATATCAGCCCT | GTTCCGATATCCGGCCG | 71 bp |
| #10 | T (196) | GATC (210) | TATAAC (224) | |
| barcode | GACGCCGTCGATAGTC | TCCGGCCCTGGACGCA | CGGCCAGTCATCGTATT | 60 bp |
| #11 | GTA (197) | (211) | GC (225) | |
| barcode | GGAGGCGCACGATTTG | CGCAGGGATCGCGACC | CGTTACGTGCGGTCTTC | 73 bp |
| #12 | TC (198) | TA (212) | GAT (226) | |
| barcode | CGCGCCGCCAATACC | TCGACAGGGAGCACTT | GGCGTATACCGGTCGA | 55 bp |
| #13 | (199) | G (213) | GTAC (227) | |
| barcode | CGAATTGCGAGTCGTT | CCGGAATCCCTGTGAT | GTCATTCGGCGGATAGA | 77 bp |
| #14 | CTATCG (200) | ACA (214) | CGTA (228) | |
| barcode | GCGCGAAAGCGTAAG | TAGTCAGGGTACAATC | CCGCCGTCGGATCGA | 71 bp |
| #15 | ATGTAC (201) | CAC (215) | (229) | |

### Bioinformatic Analysis of TaqMan™ PCR Primers and Probes

To determine whether the primers and probes designed for TaqMan™ PCR are unique for the customer-designed exonic barcodes, sequence alignment was performed with the genomes of 7 species (human, dog, mouse, rat, monkey, pig, and rabbit) using the BLAST program. The Blast searches for vector genome TaqMan™ PCR primers/probes and the Blast searches for transcript TaqMan™ PCR primers/probes were conducted separately.

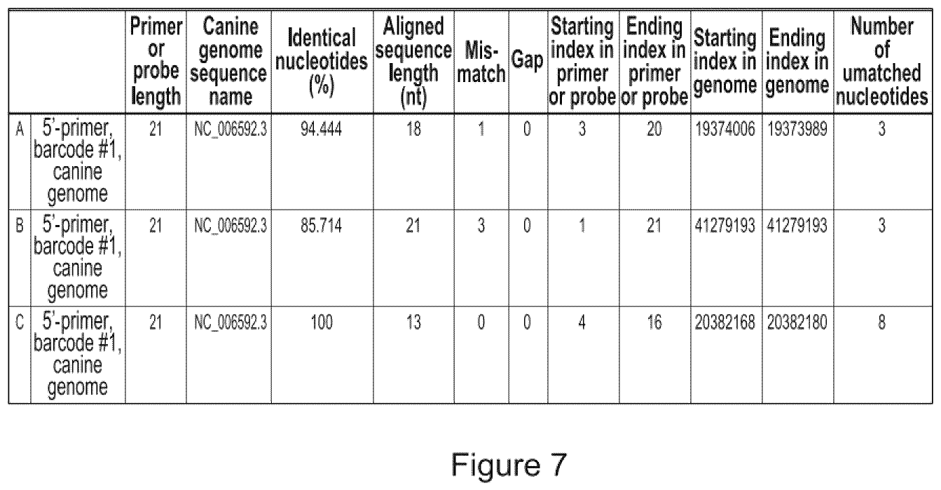

FIG. 7 shows three examples of Blast results. These are from the Blast search of the canine genome for the 5′-primers designed to evaluate the transduction efficiency (vector genome copy number) of exonic barcode #1. The length of this primer is 21 nucleotides. In example A of FIG. 7, the primer shares homology with the canine genome sequence NC_006592.3. Specifically, the nucleotides 3 to 20 of the primer share homology with the nucleotides 19374006 to 19373989 of the canine sequence (the ending number being larger than the starting number means the primer aligns to the bottom strand of the genome). The aligned sequence length is 18 nucleotides. There is one mismatch, and there is no gap. 94.44% of nucleotides are identical (1 mismatch in 18 nucleotides). In total, 3 nucleotides in the primer are not matched with the canine sequence (2 nucleotides are not aligned, and 1 nucleotide of the 18 aligned nucleotides is a mismatch).

In example B of FIG. 7, the primer shares homology with the canine genome sequence NC_006592.3. Specifically, the nucleotides 1 to 21 of the primer share homology with the nucleotides 41279193 to 41279213 of the canine sequence (the ending number being larger than the starting number means the primer aligns to the top strand of the genome). The aligned sequence length is 21 nucleotides. There are 3 mismatches, and there is no gap. 85.71% of nucleotides are identical (3 mismatches in 21 nucleotides). In total, 3 nucleotides in the primer are not matched with the canine sequence.

In example C of FIG. 7, the primer shares homology with the canine genome sequence NC_006592.3. Specifically, the nucleotides 4 to 16 of the primer share homology with the nucleotides 20382168 to 20382180 of the canine sequence. The aligned sequence length is 13 nucleotides. There is no mismatch, and there is no gap. 100% of nucleotides are identical (no mismatch in 13 nucleotides). In total, 8 nucleotides in the primer are not matched with the canine sequence (nucleotides 1-3 and 17-21).

Bioinformatic Analysis of TaqMan™ PCR Primers and Probes that Match with the Genome Sequence

A primer (or probe) sequence and the corresponding genome sequence are considered a match if they have no more than two different nucleotides. These primers (or probes) may bind DNA sequences in the genome of experimental animals. For vector genome and transcript TaqMan™ PCR, the matched primers/probes were determined via Blast search.

To determine whether these matched primers and probes can create noise signals in TaqMan™ PCR, primers and probes were identified that recognized the same gene and measured the distance between the 5′-primer and 3′-primer or between the primer (either 5′ or 3′) and the probe. The shortest distance is ˜20 kb. The amplicon size of the TaqMan™ PCR is ˜60 bp. This suggests that the primer/probe sets used in the vector genome PCR and vector transcript PCR will not generate any signal from the host genome. The results of the bioinformatic analysis suggest that the barcode TaqMan™ PCR reactions are highly specific for the barcode.

### Evaluate the Cross-Reactivity of the TagMan™ PCR Primer/Probe Sets Designed to Quantify the Vector Genome Copy Number.

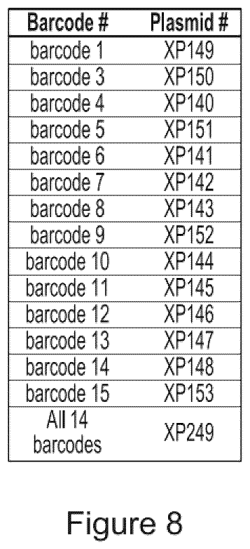

To determine whether the primer/probe set designed for one specific barcode can detect other barcodes, multiple approaches were used. In the first method, all 14 barcodes were cloned into one plasmid, and the plasmid was named the ‘all-in-one plasmid’ (XP249) (FIG. 8). In the second method, all 14 individual barcode plasmids were mixed and named ‘plasmid mixture’. Three PCR reactions were performed using the all-in-one plasmid, plasmid mixture, or barcode-specific plasmid as the PCR template. The Ct values were compared among the three PCR reactions across a broad range of template concentrations (2×10², 2×10³, 2×10⁴, 2×10⁵, 2×10⁶, 2×10⁷, 2×10⁸, and 2×10⁹).

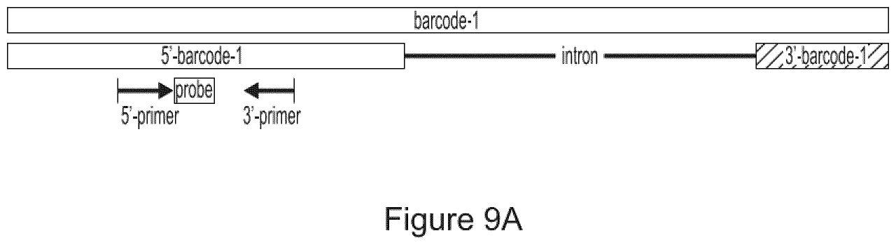

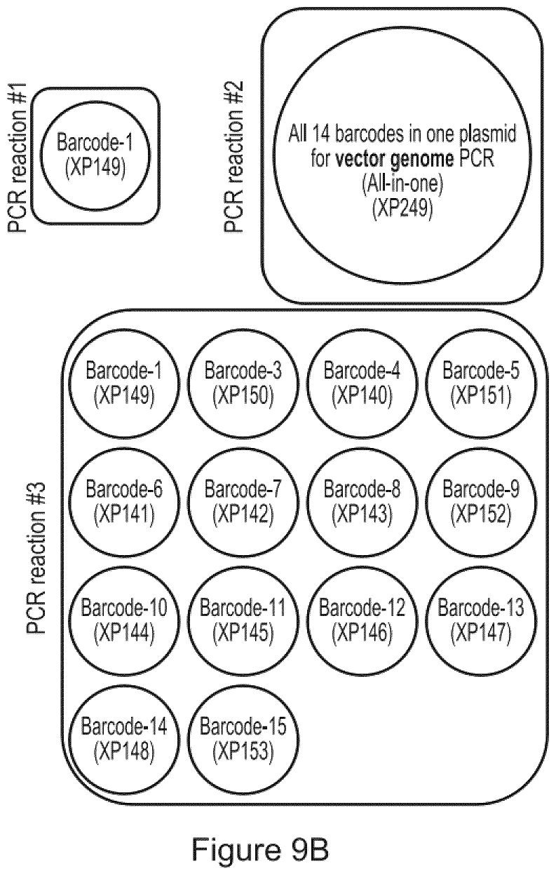

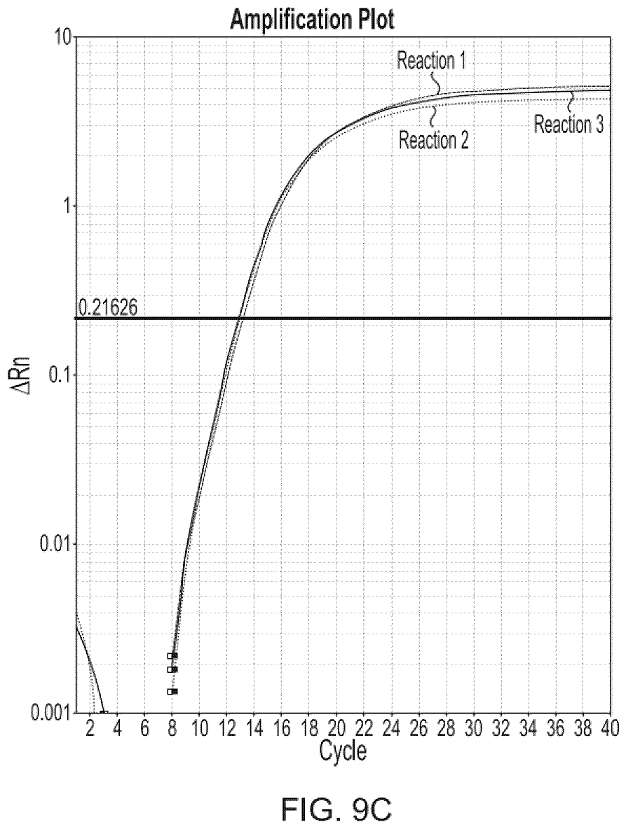

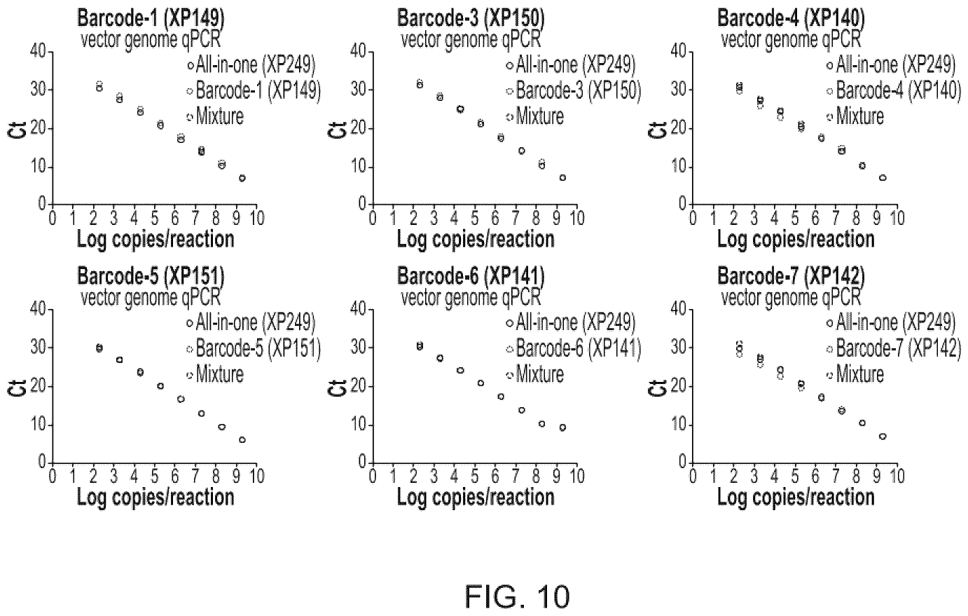

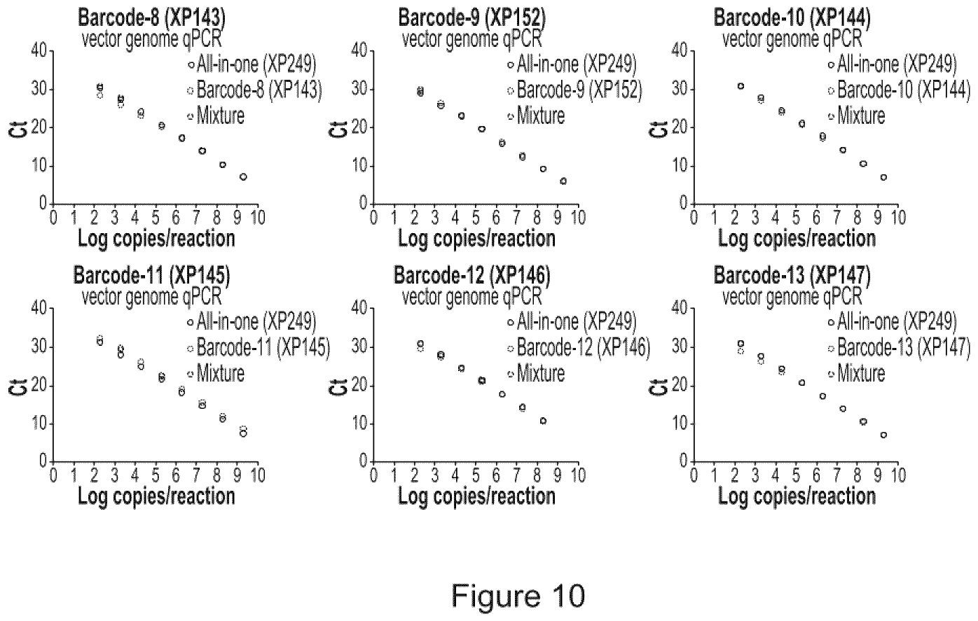

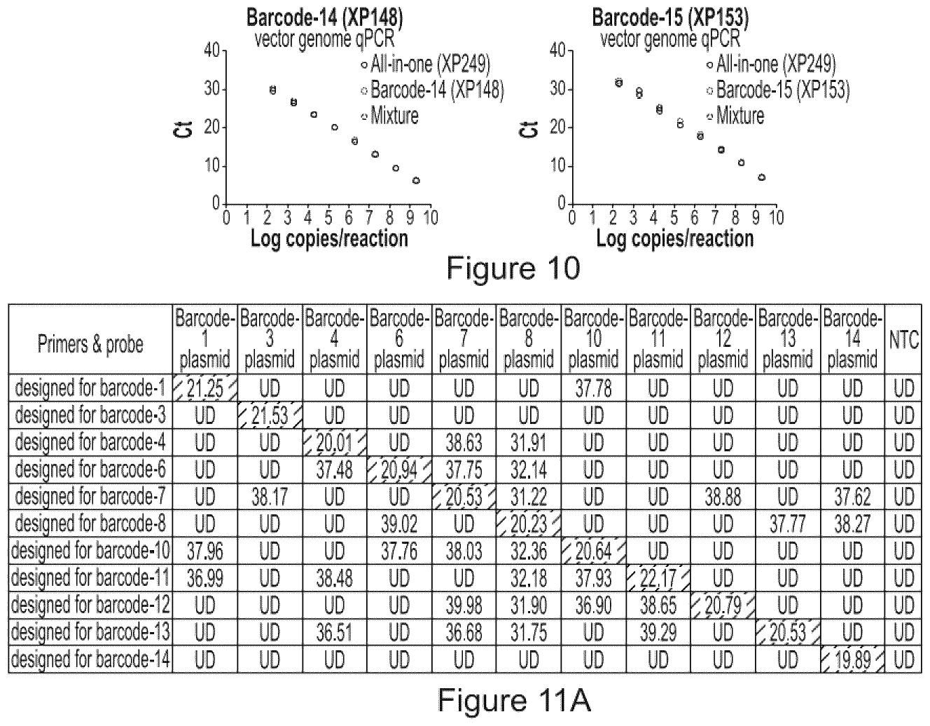

The specificity of the primer/probe sets designed to quantify the vector genome copy number was first evaluated (FIGS. 9A-9C). In this experiment, all the template plasmids carry an intron between the 5′-exon of the barcode and the 3′-exon of the barcode (FIG. 2, FIG. 9A). The results are shown in FIG. 10. Similar Ct values were obtained from all three PCR reactions at all the template concentrations. These results suggest that each barcode's primer/probe set is highly specific. The primer/probe set of one barcode does not cross-react with the remaining 13 barcodes, and there is no cross-reactivity.

### Additional Evaluation of the Specificity of the TagMan™ PCR Primer/Probe Sets Designed to Quantify the Vector Genome Copy Number

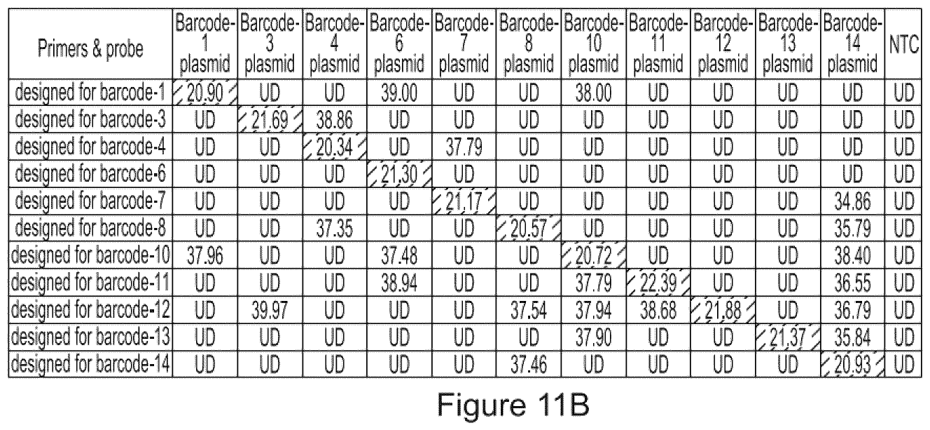

To further confirm the specificity of the primers and probes designed to quantify the vector genome copy number, PCR reactions were performed with an individual barcode plasmid as the template but using the primer/probe set designed for every barcode one by one. FIGS. 11A & 11B shows the Ct values of these PCR reactions. A Ct value of ˜21 was obtained when a barcode plasmid was amplified by its corresponding primer/probe set. However, when a barcode plasmid was amplified using primer/probe sets designed for other barcodes, the Ct values were all larger than 31 (most were larger than 35 or undetectable).

### Comparison of the Amplification Efficiency of the Primer/Probe Set Designed to Quantify the Vector Genome Copy Number

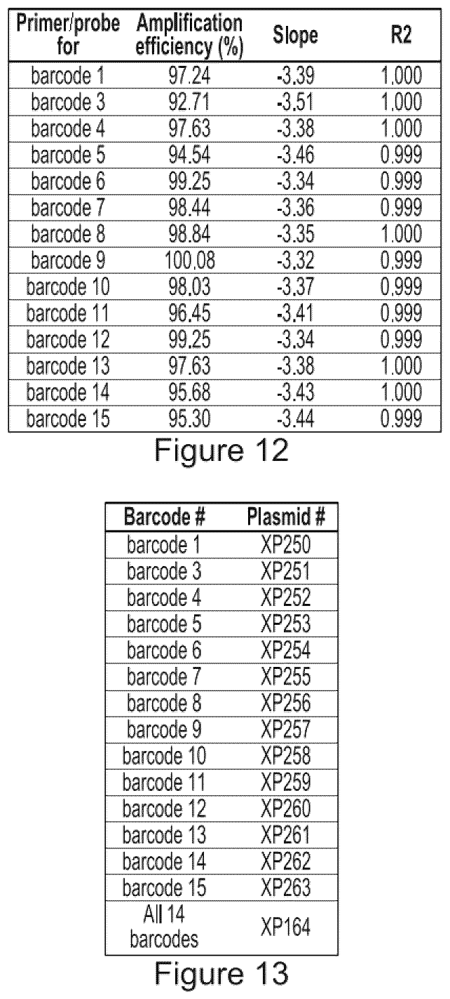

To compare the amplification efficiency of the TaqMan™ PCR reactions, a linear regression analysis was performed for PCR reactions that used the all-in-one plasmid as the template, but a barcode-specific primer/probe set in each PCR (FIG. 12). The slope was −3.39±0.05 (mean±SD). The amplification efficiency was 97.22%±2.07% (mean±SD). The small standard deviation suggests that the amplification efficiency is highly consistent among these PCR reactions.

### Evaluate the Cross-Reactivity of the TagMan™ PCR Primers and Probes Designed to Quantify the Transcript Copy Number

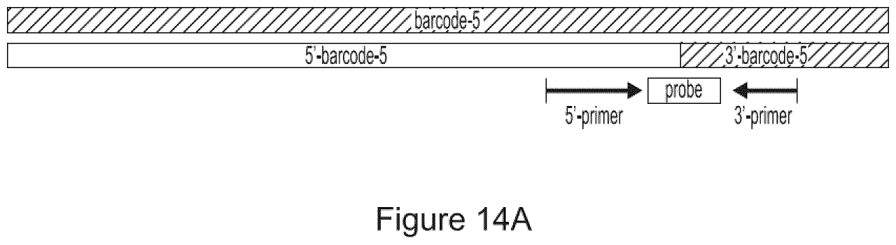

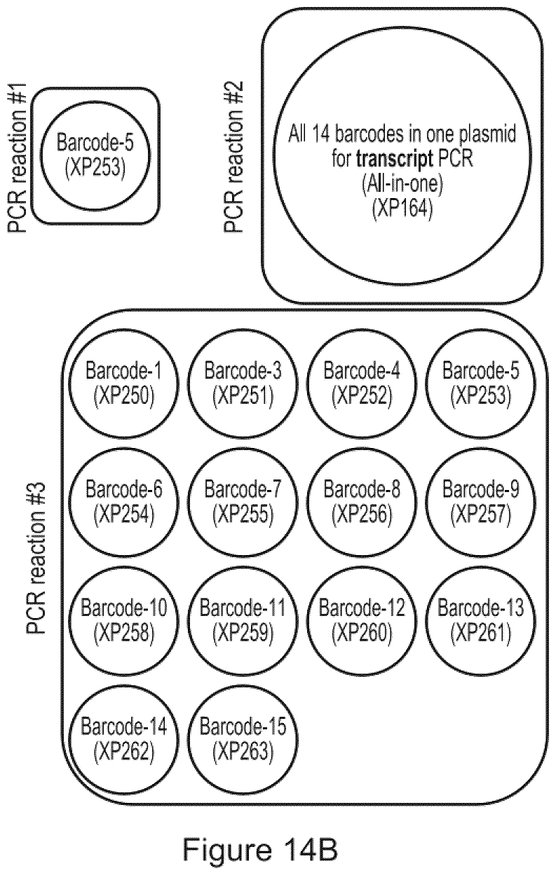

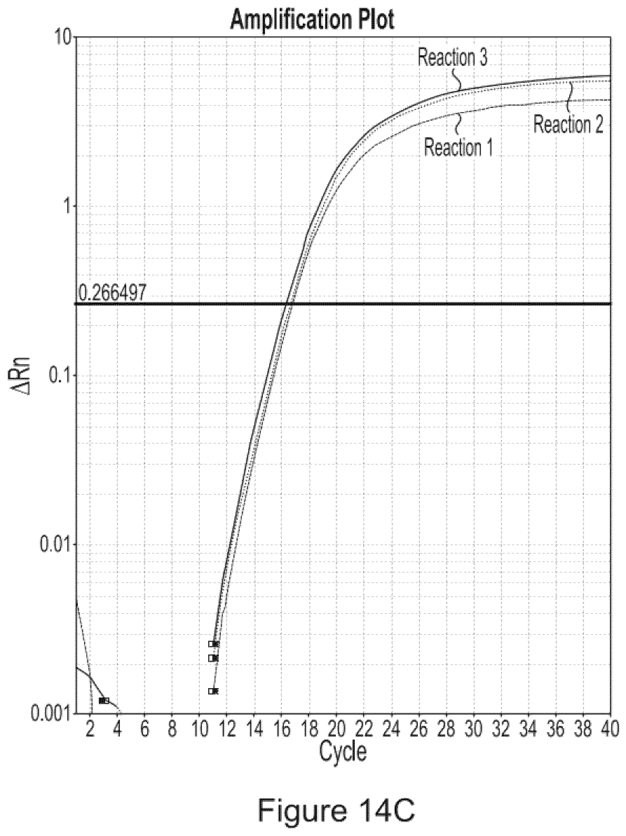

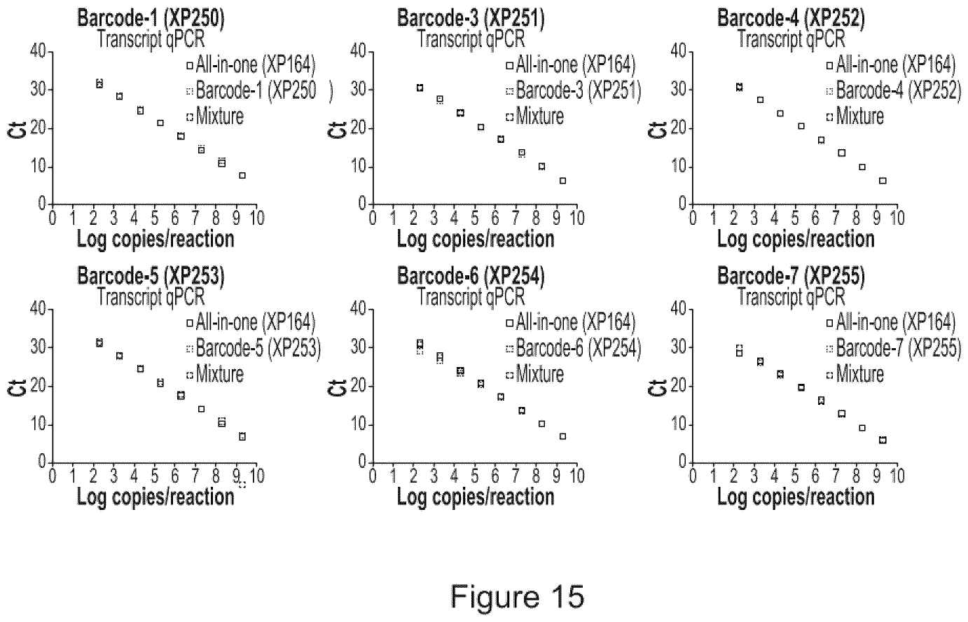

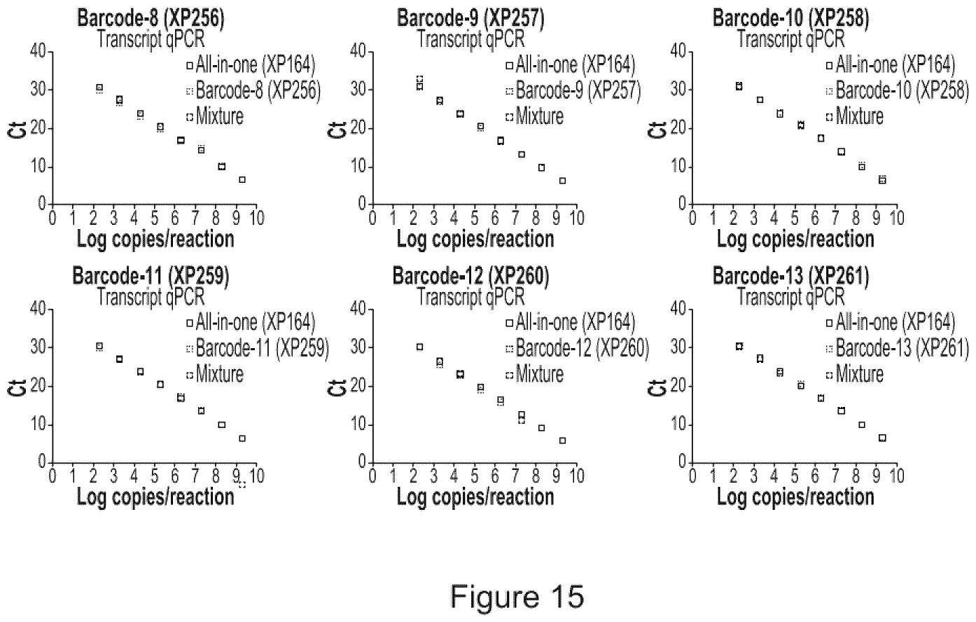

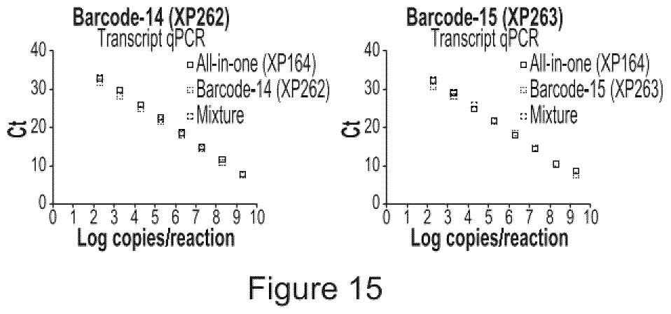

Specificity of primers and probes designed to quantify the transcript copy number was next evaluated. A series of plasmids was first made to mimic the cDNA sequence of each barcode (FIG. 13). An “all-in-one” plasmid (XP164) was also made that carries the cDNA sequence of all 14 barcodes. FIG. 14A-14C shows the strategy used to evaluate the cross-reactivity. In this experiment, the barcode cDNA plasmid (FIG. 13) was used as the PCR template. The 5′-primer is in the 5′-exon of the barcode; the probe is located at the junction of the 5′-exon and the 3′-exon, and the 3′-primer is in the 3′-exon of the barcode (FIG. 14A). The results are shown in FIG. 15. Similar to what is shown in FIG. 10, consistent Ct values were obtained from all three PCR reactions at all template concentrations. These results suggest that the primer/probe sets designed to evaluate AAV expression are highly specific. The primer/probe set design for one barcode transcript does not cross-react with the transcripts of the remaining 13 barcodes.

### Comparison of the Amplification Efficiency of the Primer/Probe Set Designed to Quantify the Transcript Copy Number

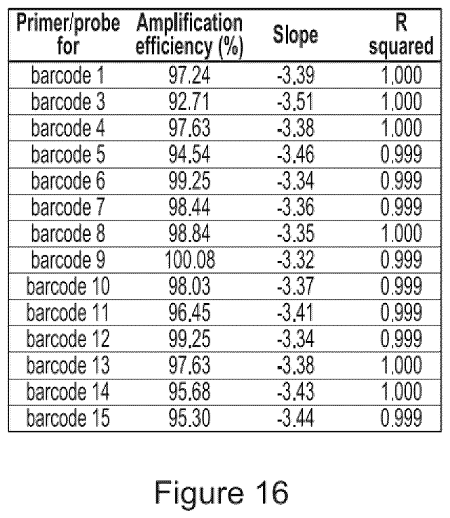

A similar study as in FIG. 12 was performed, except using the cDNA all-in-one plasmid (FIG. 16). The slope and amplification efficiencies were −3.49±0.05 and 93.41%±1.66% (mean±SD). These results suggest a consistent amplification efficiency.

### Example 4: Evaluation of the Artificial Exonic Barcode System in Mice

### AAV Capsids (Serotype) Selection

In this study, 11 different AAV capsids were compared in the mdx4cv model of Duchenne muscular dystrophy. These include AAV2, AAV8, AAV9, AAVrh74, AAV-B1, AAVNP22, AAV-NP66, AAV-S1P1, AAV-S10P1, and AAVMYO. AAV2 is the first and most studied AAV serotype. AAV2 did not support systemic muscle delivery and was used as a control. AAV8, AAV9, and AAVrh74 are currently used in systemic gene therapy for inherited neuromuscular diseases. AAV-B1 is engineered by the Miguel Sena-Esteves lab. It previously showed superior transduction in mouse muscle and central nervous system. AAV-NP22 and AAV-NP66 are developed by the Mark Kay lab. These two capsids previously showed significantly increased transduction in human and rhesus skeletal muscle fiber. AAV-S1P1 and AAV-S10P1 are generated in the Dirk Grimm lab. These capsids previously showed increased potency and specificity for systemic delivery to muscle and de-targeting from the liver. AAVMYO is developed in the Dirk Grimm lab, too. AAVMYO exceeded AAV-S1P1 and AAV-S10P1 in muscle targeting and liver detargeting. AAV-KP1 is generated in the Mark Kay lab. This capsid transduced mouse and human liver at very high levels and was used as an additional control.

### Check the Cross-Reactivity of the PCR Primer/Probe Sets in AAV Viruses

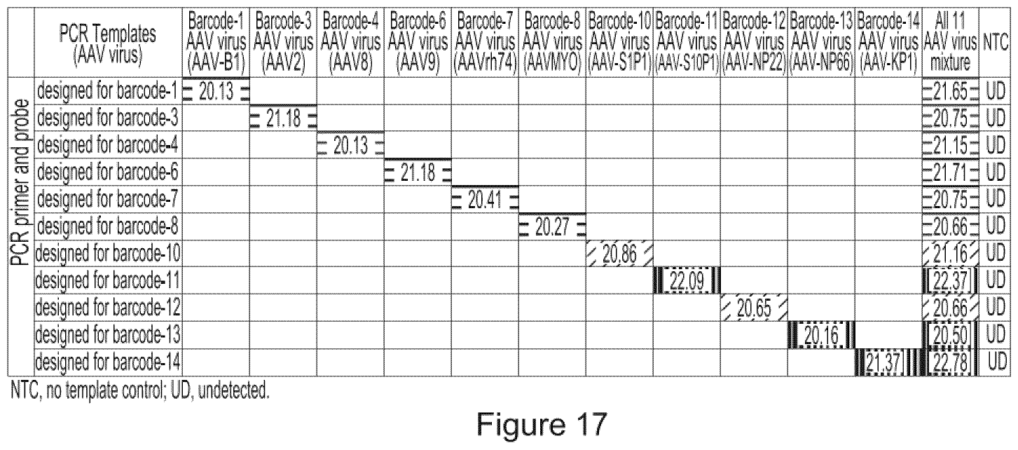

The exonic barcode system was packaged with the above-listed 11 AAV capsids, and the barcoded AAV viruses were purified. The cross-reactivity of the primer/probe sets designed to quantify the vector genome copy number was first checked. It was shown that these primer/probe sets were highly specific to their corresponding barcodes when plasmids were used as the template (FIGS. 9A-9C, 10, and 11A & 11B). Consistently, cross-reactivity was not detected among different primer/probe sets when AAV virus was used as the template (FIG. 17). Similar Ct values were obtained when a primer/probe set was used to amplify its corresponding barcoded virus or the virus mixture.

In Vivo Study in mdx4cv Mice

The study was performed in 4-m-old male mdx4cv mice by tail vein injection. The barcoded virus mixture was delivered at a dose of either 3×10¹² vg/kg/AAV capsid (3.3×10¹³ vg/kg total AAV) or 1×10¹³ vg/kg/AAV capsid (1.1×10¹⁴ vg/kg total AAV) (n=3 mice/dose). Tissues were harvested one month later.

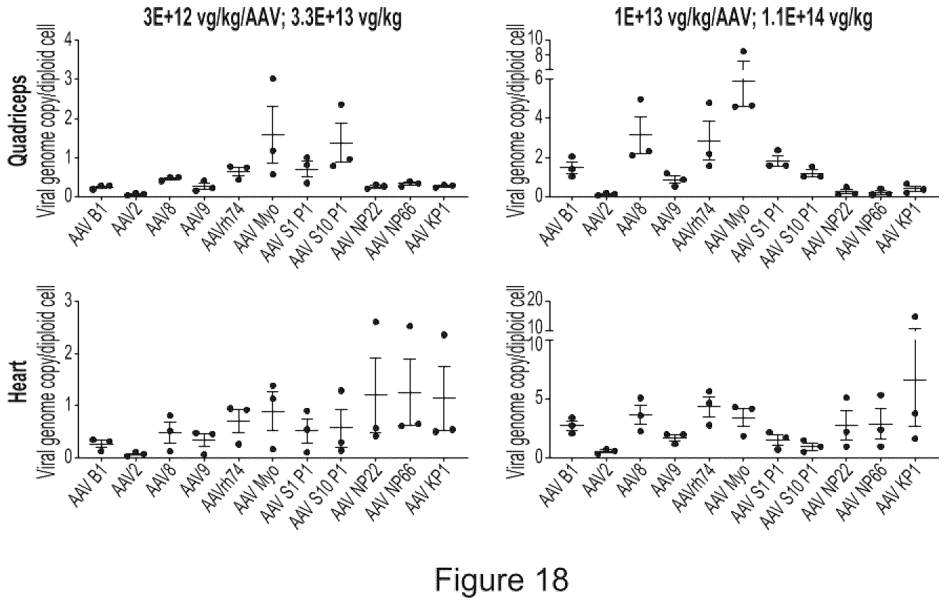

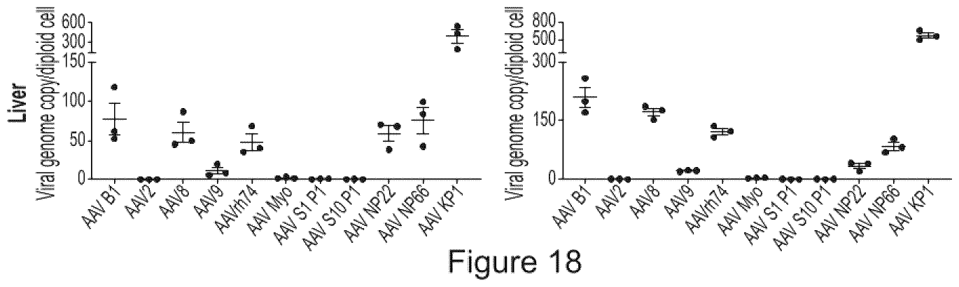

FIG. 18 shows the results of vector genome copy number quantification for each AAV capsid. Consistent results were obtained for both doses, although the trend was clearer in the high-dose group. In skeletal muscle (quadriceps), AAVMYO showed the highest vector genome copies. AAVB1, AAV8, AAV-S1P1, and AAV-S10P1 also showed good skeletal muscle delivery. AAV2 showed the lowest transduction efficiency in the heart. AAVB1, AAV8, AAV9, AAVrh74, AAVMYO, AAV-S1P1, AAV-NP22, AAV-NP66, and AAV-KP1 showed good transduction in the heart. AAV-KP1 showed the highest vector genome copies in the liver, followed by AAVB1, AAV8, AAVrh74, AAV-NP66, and AAV-NP22. AAV2, AAV9, AAVMYO, AAV-S1P1, and AAVS10P1 had minimal transduction in the liver. These results are, in general, consistent with the literature.

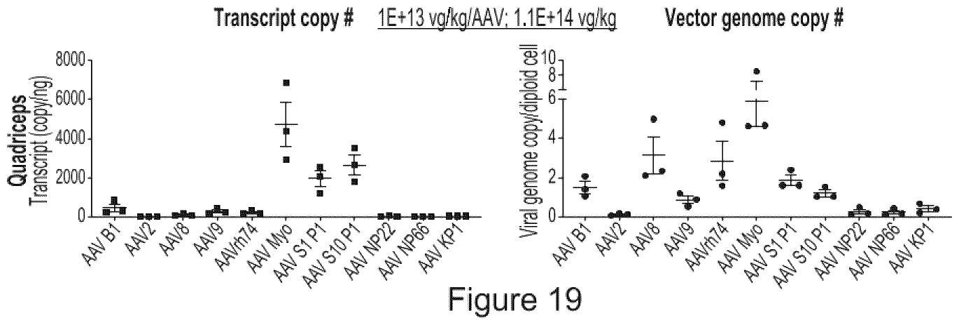

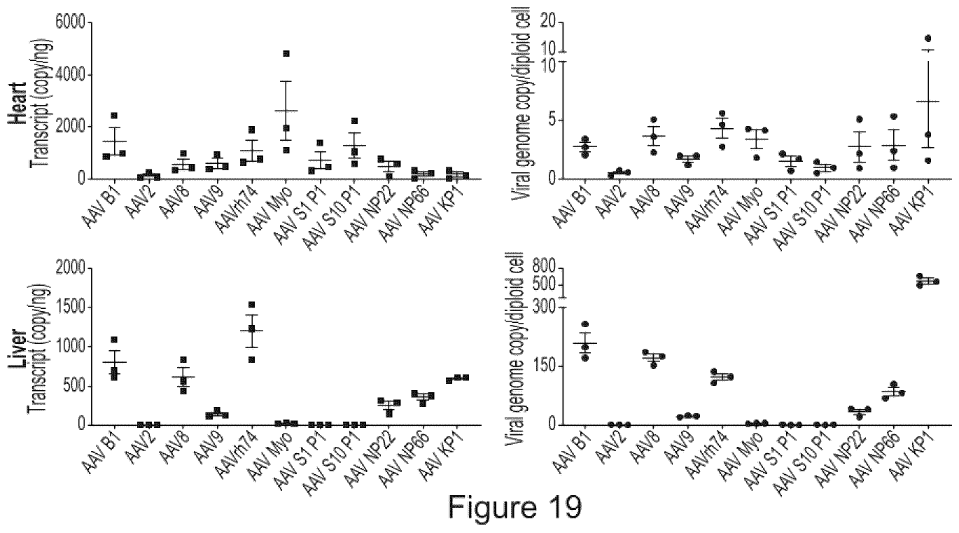

FIG. 19 shows the results of transcript copy number quantification for each AAV capsid from the high-dose group. In skeletal muscle (quadriceps), AAVMYO showed the highest expression, followed by AAV-S1P1 and AAV-S10P1. AAV2, AAV-NP22, AAV-NP66, and AAV-KP1 showed nominal expression, consistent with their low vector genome copy numbers. Surprisingly, several capsids with high vector genome copy numbers (AAVB1, AAV8, AAV9, AAVrh74) showed poor expression, suggesting defective intracellular processing when these capsids are used for systemic muscle gene delivery. In the heart, high expression was detected for AAVB1 and AAVMYO, followed by AAVrh74, AAV-S10P1, AAV-S1P1, AAV8, and AAV9. AAV2, AAV-NP66, and AAV-KP1 showed very low (or no) expression in the heart. This is intriguing since these capsids resulted in relatively good transduction (vector genome copy number). In the liver, AAVrh74, AAVB1, and AAV8 resulted in the highest expression, followed by AAV-KP1, AAV-NP22, and AAV-NP66. Transduction data (vg copy number) and expression data (transcript copy number) were consistent for all AAV capsids except AAV-KP1. The expression level was lower than the transduction efficiency for AAV-KP1.

In summary, this pilot mouse study highlighted the importance of evaluating both the vg copy number (for transduction) and transcript copy number (for expression). While most times, these were consistent, there are many exceptions. Further, AAV-mediated gene transfer could be greatly influenced by the target tissue or organ. For example, AAV8 resulted in good transduction but the poor expression in skeletal muscle. However, transduction and expression were consistent in the liver for AAV8.

### Example 5: Evaluation of the Exonic Barcode System in Dogs

### Experimental Plan

The same 11 capsids investigated in mdx4cv mice were used in the dog study. AAV mixture was delivered by intravenous injection to one 1-week-old puppy at the dose of 3.6×10¹² vg/kg/AAV (4×10¹³ vg/kg total AAV) and one 1-month-old dog at the dose of 5.5×10¹² vg/kg/AAV (6.1×10¹³ vg/kg total AAV). Both were carrier dogs (they did not have muscular dystrophy). Tissues were harvested at 3 weeks after injection. The vector genome copy number and the transcript copy number were quantified from five skeletal muscles (diaphragm, triceps, biceps femoris, extensor digitorum longus, and vastus lateralis), heart, and liver.

### Quantification of the Vector Genome Copy Number (Transduction Efficiency)

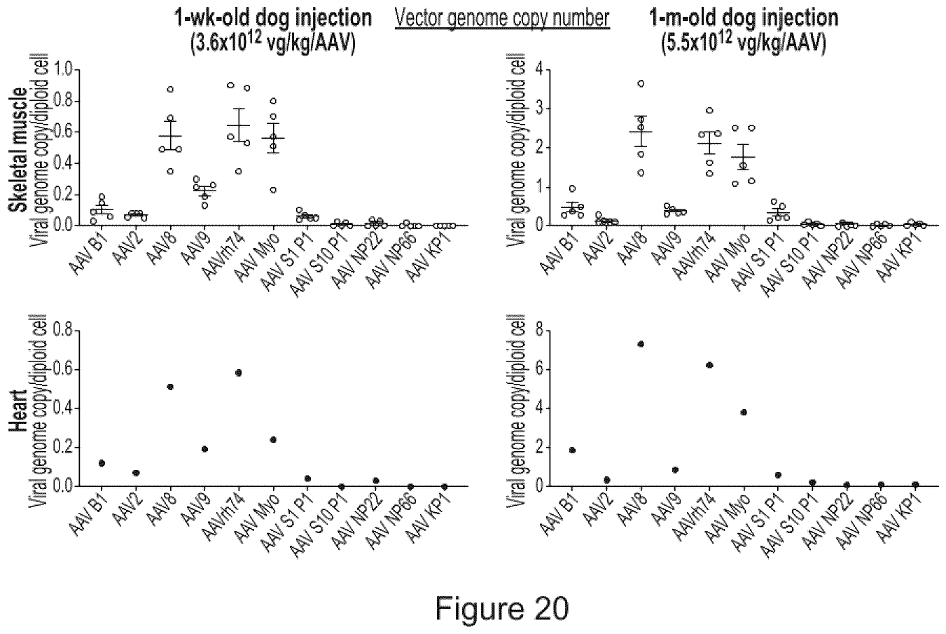

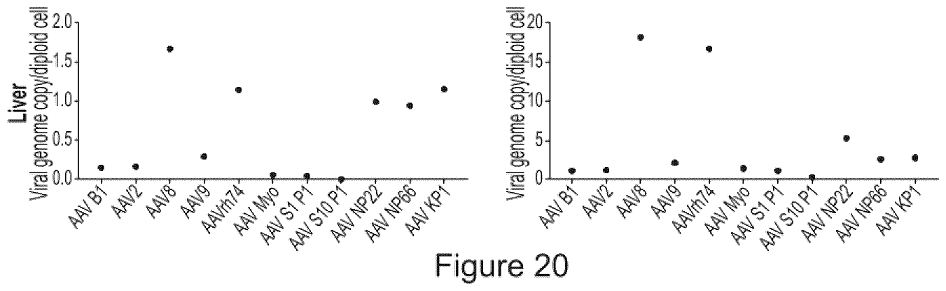

FIG. 20 shows the results of vector genome copy number quantification for each AAV capsid. Consistent trends were obtained from both dogs. In skeletal muscle and heart, AAV8, AAVrh74, and AAVMYO had the highest vector genome copies, followed by AAVB1, AAV9, and AAV-S1P1. AAV8, AAVrh74, AAV-NP22, AAV-NP66, and AAV-KP1 had a high vector genome copy number in the liver.

### Quantification of the Transcript Copy Number (Expression Level)

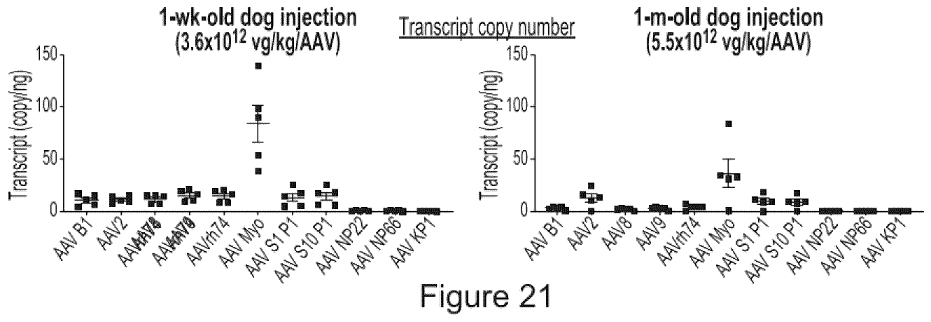

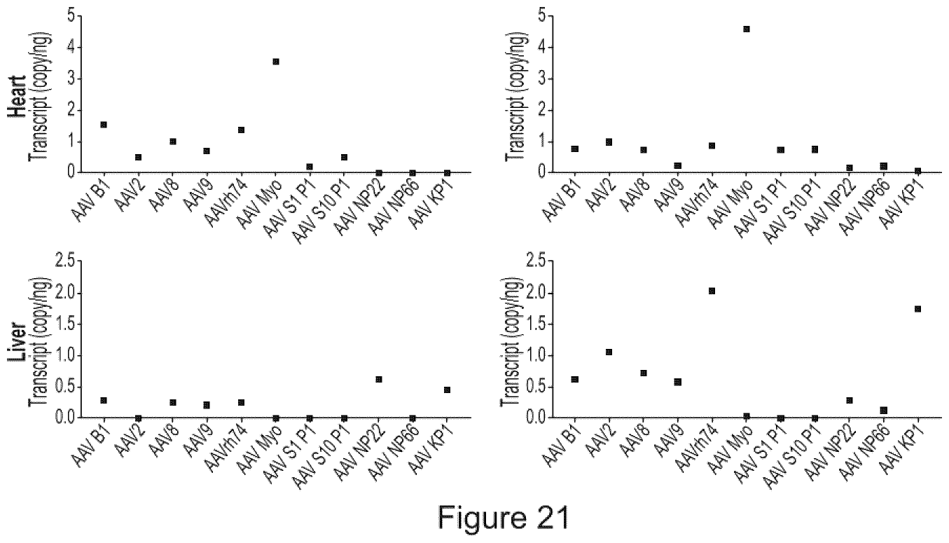

FIG. 21 shows the results of vector transcript copy number quantification for each AAV capsid. Consistent trends were obtained from both dogs. In skeletal muscle and heart, AAVMYO resulted in the highest expression. The other capsids showed low or no expression in skeletal muscle. AAVB1, AAV2, AAV9, AAVrh74, AAV-S1P1, and AAV-S10P1 showed moderate expression in the heart. In the liver, AAVB1, AAV8, AAV9, AAVrh74, AAVNP22, and AAV-KP1 had high expression. AAV2 data was inconsistent between the two dogs. Importantly, AAVMYO, AAV-S1P1, and AAVS10P1 showed no expression in the liver.

### Comparison of AAV Transduction and AAV-Mediated Expression in Dogs

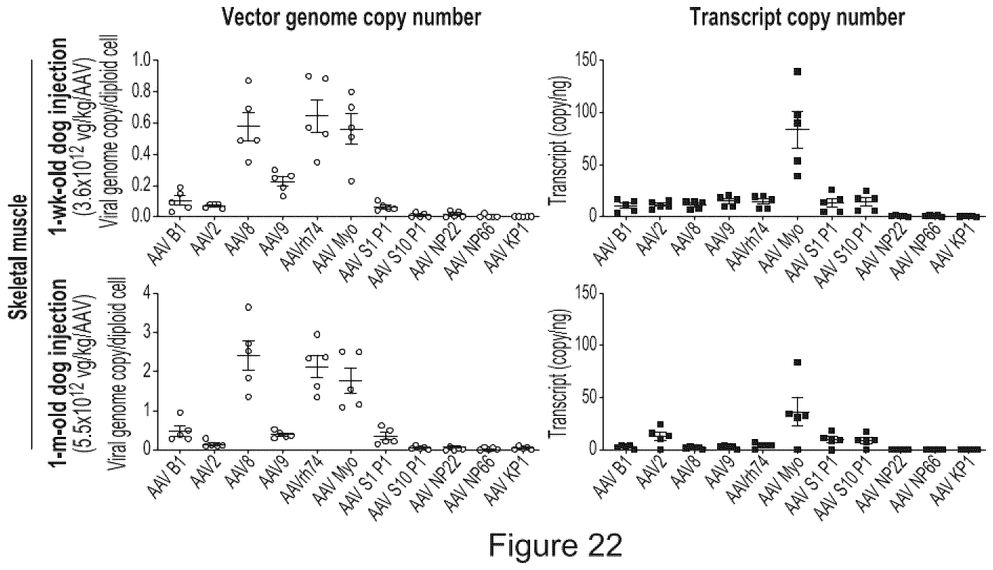

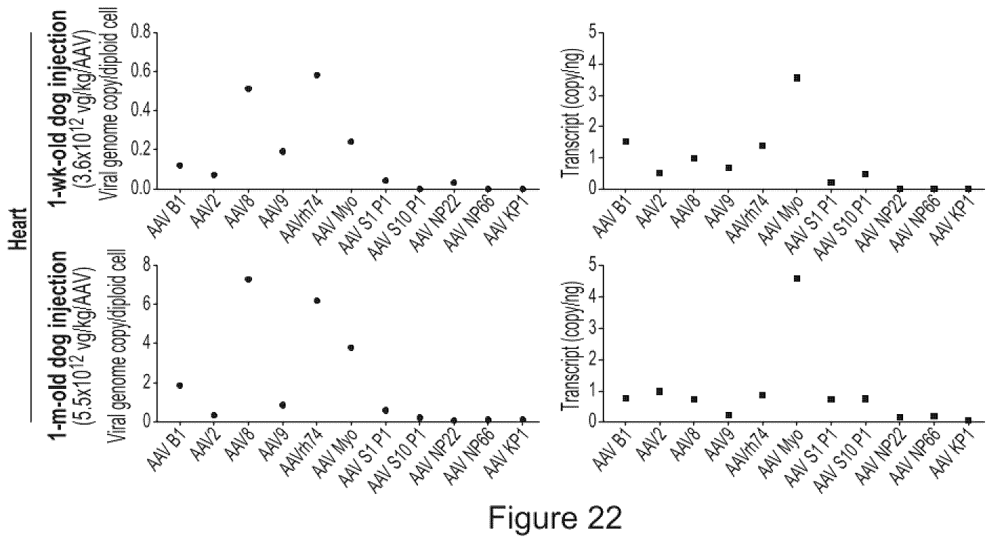

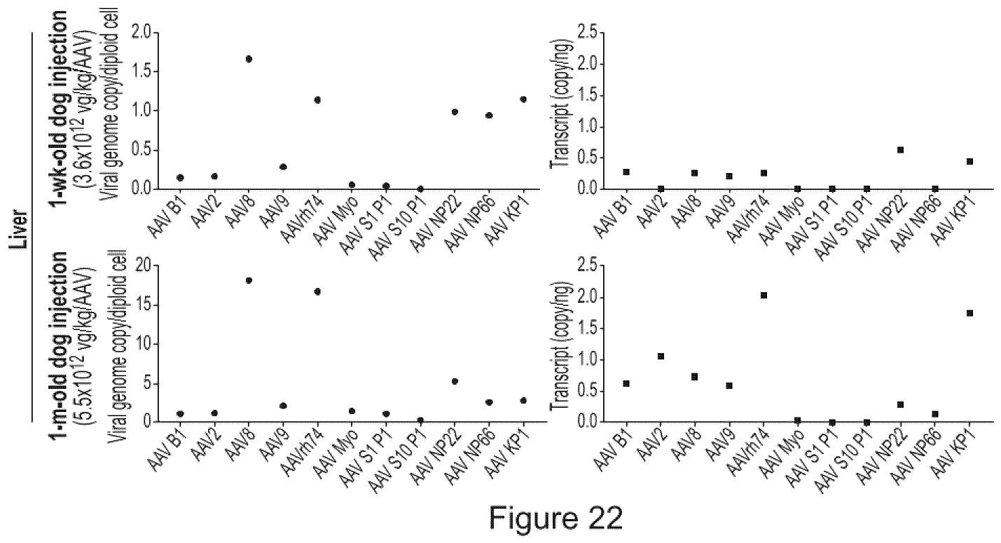

The correlation between AAV transduction and AAV expression was compared for both dogs (FIG. 22). AAVMYO showed good transduction (vg copy number) and expression (transcript copy number) in skeletal muscle. AAVB1, AAV2, AAV9, AAV-S1P1, AAV-S10P1, and AAV-KP1 showed moderate to low transduction and minimum expression. Surprisingly, AAV8 and AAVrh74 showed a high vector genome copy number (comparable to that of AAVMYO), but their expression was minimal (similar to AAVB1, AAV2, AAV9, AAVS1P1, and AAV-S10P1). AAV-NP22, AAV-NP66, and AAV-KP1 have minimal transduction and minimal expression.

In the heart, AAV8 and AAVrh74 showed the highest vector genome copy number (the highest transduction efficiency) but only moderate expression (transcript copy number). In contrast, AAVMYO had a moderate transduction efficiency but the highest expression. AAVB1, AAV2, AAV9, and AAV-S1P1 showed moderate transduction and moderate expression. AAV-S10P1 had very low transduction but a moderate expression. AAV-NP22, AAV-NP66, and AAV-KP1 have minimal transduction and minimal expression.

In the liver, AAV8 showed the highest vector genome copy number but only moderate expression. AAVrh74 had a high copy number, but the high expression was only found in the 1-m-old dog. AAVrh74 expression was similar to AAV8 in the 1-week-old puppy. AAV-NP22, AAV-NP66, and AAV-KP1 showed good (in 1-week-old puppy) and moderate (in 1-m-old dog) transduction. However, only AAV-KP1 showed high expression. AAV-NP-66 had a nominal expression.

### Example 6: Summary of In Vivo Study in Mice and Dogs

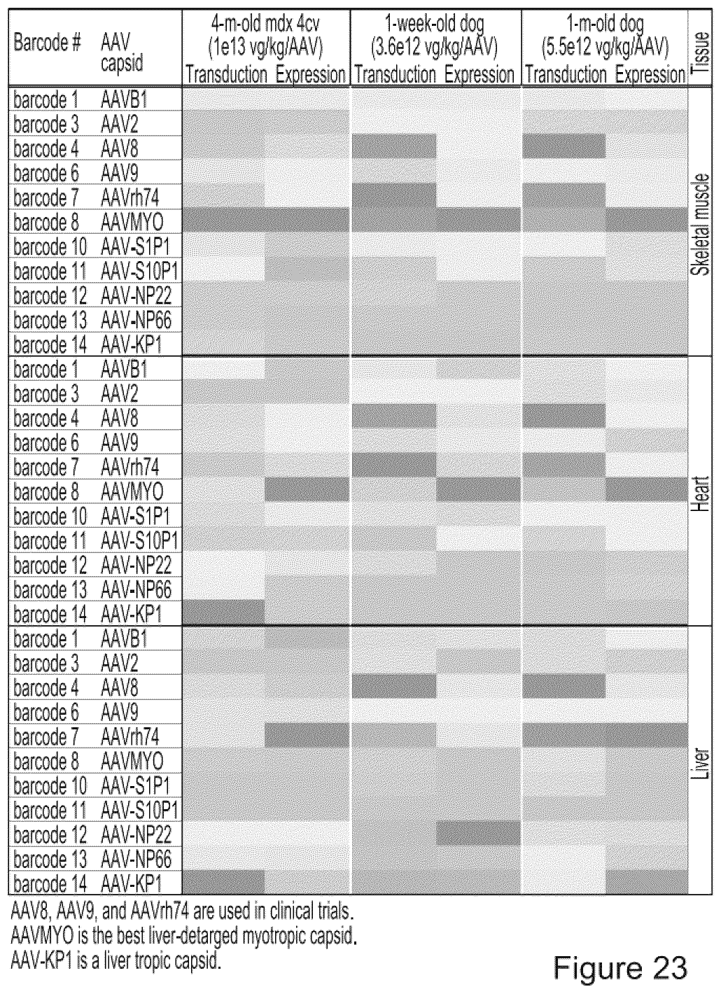

FIG. 23 summarizes transduction (vector genome copy number) and expression (transcript copy number) data from mdx4cv mice and dogs. Consistent with the literature, AAVMYO showed the best performance in muscle and heart and was detargeted from the liver. Two other myotropic AAV capsids developed in the Dirk Grimm lab (AAV-S1P1, and AAV-S10P1) also showed good skeletal muscle performance and were detargeted from the liver. AAVB1 has good transduction in skeletal muscle and heart but was not detargeted from the liver. AAV-KP1 showed the best performance in the liver and was detargeted from skeletal muscle. AAV-NP22 and AAV-NP66 were shown to have enhanced performance in human and non-human primate muscle fiber. The data disclosed herein suggest that these two capsids are not good in murine and canine muscles.

AAV8, AAV9, and AAVrh74 are currently used in clinical trials to treat inherited neuromuscular diseases. They showed good performance in muscle tissues, but they also had strong liver targeting (especially AAVrh74 and AAV8). This is consistent with the liver toxicity observed in human trials.

1. An exonic barcode comprising a nucleotide sequence comprising, from 5′ to 3′, a 5′ barcode, an intron, and a 3′ barcode,
wherein the 5′ barcode is at least 50 bp long;
wherein the 3′ barcode is at least 50 bp long;
wherein at least one of the 5′ barcode and 3′ barcode is at least 150 bp long;
wherein the 5′ barcode and 3′ barcode have minimum homology with human, monkey, pig, dog, rabbit, mouse, and rat genomes and have minimum homology with each other;
wherein minimum homology is defined by a BLAST search E-value of greater than 0.05;
wherein the exonic barcode does not have alternative splice sites;
wherein the 5′ barcode and 3′ barcode each has no repeated sub-fragments longer than 6 nucleotides;
wherein the 5′ barcode and 3′ barcode each does not contain a target sequence of any restriction enzyme used in cloning the exonic barcode or any sequence identical to the target sequence except for one different nucleotide;
wherein the 5′ barcode and 3′ barcode each do not contain four identical nucleotides in a row;
wherein the 5′ barcode ends with a “CAG” nucleotide sequence and does not contain a “GGT” nucleotide sequence; and
wherein the 3′ barcode starts with a “G” nucleotide and does not contain an “AAG” nucleotide sequence.

2. The exonic barcode of claim 1, wherein the intron is a pCI intron.

3. The exonic barcode of claim 1, wherein the 5′ barcode has a maximum aligned identical sequence length with the human and/or dog genome of equal to or less than 21 and/or the 3′ barcode has a maximum aligned identical sequence length with the human and/or dog genome of equal to or less than 18.

4. (canceled)

5. The exonic barcode of claim 1, wherein the 5′ barcode and 3′ barcode have no identical sequence fragments equal to or greater than 8 nucleotides.

6. The exonic barcode of claim 1, wherein the nucleotide sequence is at least 300 nucleotides long.

7. The exonic barcode of claim 1, wherein the human genome is a Homo sapiens genome, the monkey genome is a Macaca mulatta genome, the pig genome is a Sus scrofa genome, the dog genome is a Canis lupus familiaris genome, the rabbit genome is a Oryctolagus cuniculus genome, the mouse genome is a Mus musculus genome, and/or the rat genome is a Rattus norvegicus genome.

8. The exonic barcode of claim 1, wherein the nucleotide sequence comprises any one of SEQ ID NO: 31 AND 33-45.

9. A synthetic reporter gene comprising a nucleotide sequence comprising a reporter coding sequence and the exonic barcode of claim 1.

10. (canceled)

11. A library of exonic barcodes comprising two or more exonic barcodes according to claim 1, wherein there are no duplicated fragments longer than eight nucleotides shared among any 5′ barcode, any 3′ barcode, and any 5′ barcode and 3′ barcode.

12. A method of generating an exonic barcode library, the method comprising:
a) independently generating a 5′ DNA fragment library and a 3′ DNA fragment library each comprising at least 200,000 20-nucleotide-long random DNA fragments;
wherein each random DNA fragment in the 5′ DNA fragment library and the 3′ DNA fragment library has no repeated sub-fragment longer than 6 nucleotides, each fragment does not contain a target sequence of any restriction enzyme to be used in cloning the exonic barcode library or any sequence identical to the target sequence except for one different nucleotide, and each fragment does not contain four identical nucleotides in a row;
wherein each random fragment in the 5′ DNA fragment library does not contain the sequence “GGT;”
wherein each fragment in the 3′ DNA fragment library does not contain the sequence “AGG”;
b) generating a refined 5′ DNA fragment library by removing DNA fragments from the 5′ DNA fragment library that have a maximum aligned identical sequence length of greater than 21 nucleotides with human and/or dog genomes or that share sequence fragment lengths of greater than 8 nucleotides with any other fragments of the 5′ and/or 3′ DNA fragment libraries; and
generating a refined 3′ DNA fragment library by removing DNA fragments from the 3′ DNA fragment library that have a maximum aligned identical sequence length of greater than 18 nucleotides with human and/or dog genomes or that share sequence fragment lengths of greater than 8 nucleotides with any other fragments of the 5′ and/or 3′ DNA fragment libraries;
c) generating a 5′ exonic barcode library comprising at least 500,000 150 nucleotide-long 5′ barcodes by combining eight 20-nucleotide-long random DNA fragments from the refined 5′ DNA fragment library and removing the last 10 nucleotides and generating a 3′ exonic barcode library comprising at least 500,000 50-nucleotide-long 3′ barcodes by combining three 20-nucleotide-long random DNA fragments from the refined 3′ DNA fragment library and removing the last 10 nucleotides;
wherein each barcode of the 5′ exonic barcode library or the 3′ exonic barcode library has no repeated sub-fragment longer than 6 nucleotides, the 5′ barcode and 3′ barcode each do not contain a target sequence of any restriction enzyme used in cloning the exonic barcode or any sequence identical to the target sequence except for one different nucleotide, and each barcode does not contain four identical nucleotides in a row;
wherein each barcode in the 5′ exonic barcode library ends with a “CAG” nucleotide sequence and does not contain a “GGT” nucleotide sequence;
wherein each barcode in the 3′ exonic barcode library starts with a “G” nucleotide and does not contain an “AAG” nucleotide sequence;
d) generating a refined 5′ exonic barcode library and a refined 3′ exonic barcode library by removing any barcodes that have a maximum aligned identical sequence length of greater than 8 with any other barcode in either library and removing any barcodes that share homology with the human, monkey, pig, dog, rabbit, mouse, and/or rat genomes, wherein sharing homology is defined by a BLAST search E-value of 0.05 or less; and
e) generating the exonic barcode library comprising exonic barcodes, wherein each exonic barcode is generated by combining, from 5′ to 3′, one barcode from the refined 5′ exonic barcode library, an intron, and one barcode from the refined 3′ exonic barcode library, and wherein any exonic barcode that comprises an alternative splice site is removed from the exonic barcode library.

13. The method of claim 12, wherein the exonic barcode has a GC content of from about 50% to about 60%.

14. The method of claim 13, wherein the 5′ barcode and 3′ barcode each do not contain “TTAATTAA (SEQ ID NO: 237),” “GCTAGC (SEQ ID NO: 238),” or any sequence identical to “TTAATTAA (SEQ ID NO: 237)” or “GCTAGC (SEQ ID NO: 238)” except for one different nucleotide.

15. The method of claim 12, wherein each barcode from the 5′ exonic barcode library and the refined 3′ exonic barcode library is used at most once in generating the exonic barcodes of the exonic barcode library in step e).

16. The method of claim 12, wherein step d) comprises one or more of: removing any barcode in the 5′ exonic barcode library that has a maximum aligned identical sequence length with the human and/or dog genome of greater than 21 or removing any barcode in the 3′ exonic barcode library that has a maximum aligned identical sequence length with the human and/or dog genome of greater than 18.

17. (canceled)

18. The method of claim 12, wherein the human genome is a Homo sapiens genome, the monkey genome is a Macaca mulatta genome, the pig genome is a Sus scrofa genome, the dog genome is a Canis lupus familiaris genome, the rabbit genome is a Oryctolagus cuniculus genome, the mouse genome is a Mus musculus genome, and the rat genome is a Rattus norvegicus genome.

19. A method of screening for efficiency of transformation and/or expression of one or more genetic constructs in a subject, the method comprising:
a) transforming the one or more genetic constructs into the subject, wherein each of the one or more genetic constructs comprises a nucleotide sequence encoding a different protein of interest conjugated to a different exonic barcode of claim 1;
b) harvesting cells from the subject;
c) performing on the cells one or more methods selected from the group consisting of real-time PCR, high-throughput sequencing, conventional PCR, Southern blotting, Northern blotting, and in situ hybridization; and
d) evaluating the one or more methods for the relative amounts of genome copies and/or transcript copies of the one or more genetic constructs to determine the efficiency of transformation and/or expression.

20. The method of claim 19, wherein the transformation is selected from the group consisting of a stable integration, via transfection and via a virus.

21. (canceled)

22. (canceled)

23. The method of claim 20, wherein the virus is AAV.

24. The method of claim 23, wherein the protein of interest of the one or more genetic constructs each comprises a different AAV capsid.

25. (canceled)

26. The method of claim 19, wherein the method further comprises harvesting cells from more than one tissue of the subject in step b) and performing steps c) and d) separately on the cells from each tissue to screen for efficiency of transformation and/or expression separately in each tissue.

27. (canceled)

28. (canceled)

29. (canceled)

30. (canceled)

31. (canceled)

32. (canceled)


SEQUENCE LISTING (236 sequences)

SEQ ID NO: 1 - DNA, 150 bp, synthetic construct
  source 1..150: mol_type = other DNA
ccgcgtacccgtgatgactatcgcgccgttataccacgcaacggccatgctacgacgtatagttcgcacgcgatactcgaggcgttgcgccattgacgtttcgcgtggcgctattagtccgatccgcgacgactagtagcgtagagacag

SEQ ID NO: 2 - DNA, 150 bp, synthetic construct
  source 1..150: mol_type = other DNA
tcgtcgctacgaacgcaacgtagcgcgacataccggcaatgcccgtaaacggcatcgtatagcgaatccgatcagtcgtcctgttaccgacgcgcaatactacccgcgtcactatacgctttagacgcctcgccgttactttattcgcag

SEQ ID NO: 3 - DNA, 150 bp, synthetic construct
  source 1..150: mol_type = other DNA
cgcaaagccgaagttacgcggattgtcgacccgcggctttcggacatttcgcgccgactatcgttcggcgctcgttattcgtaggcgtaatgccgagttgcgaacgacgcaagtacgcctaacgcccgtctaccgtacgtgtcgccgcag

SEQ ID NO: 4 - DNA, 150 bp, synthetic construct
  source 1..150: mol_type = other DNA
cagcggaacgcgtacagtagccgtatgcgcgtcgcttagacgtttggcgaacgaactcgagtaacacgttcgcgttgaccgattcgtggcgcatcgcctaataatgcgtagtcggcggcgagttgtcgacgcgcccaatatctatgacag

SEQ ID NO: 5 - DNA, 150 bp, synthetic construct
  source 1..150: mol_type = other DNA
cacggaccactaatcgggaccgcagacgaacccgttcgaacaagcgtcgtcggagtaacccacgcgaattcgatggccgaacgttgacgacgctcgacattacgctgcgcgacgtattgtgcgtagcgtaagtcgtttcgtacacggcag

SEQ ID NO: 6 - DNA, 150 bp, synthetic construct
  source 1..150: mol_type = other DNA
cgcgtacttccgactaaccgttccgtaacatacgcccgagcggcgcactacgatatagactggcgcgatcgtccatcgatgtagcgcgtggatgcatcgtttagctcgacaccggcgtgtgtcgaacgtcgcataacggacccgttgcag

SEQ ID NO: 7 - DNA, 150 bp, synthetic construct
  source 1..150: mol_type = other DNA
tctagtgcgacgcgaacgtttgccgtaccgtagacgagacccgttctacgatcgcctatcgatccggcataccgagagtcctcgtcgcagtacgcactttctcggcgcgattgtagcgttgtaatcgcgtgcgggcgaatagtggcgcag

SEQ ID NO: 8 - DNA, 150 bp, synthetic construct
  source 1..150: mol_type = other DNA
ctgttcgtaccacacgtcgagtccgcgtgatacgtttcgacgatctatacgcgcgccacttggacgcgtttaacgcccaccgagtacgattacgccggacttcgcgatatgcggacatcgaagcgtgcgtccgtatcgagcataaagcag

SEQ ID NO: 9 - DNA, 150 bp, synthetic construct
  source 1..150: mol_type = other DNA
cgctgatacgacggataccgaccattactccgcgaggcgtcgcccgattagtgcatacggcgacccgccgacatcgttaagacgcaaattcgcgctacgggatgagcgacagcgttgcgaagtacgtccggagtcgtagataacggccag

SEQ ID NO: 10 - DNA, 150 bp, synthetic construct
  source 1..150: mol_type = other DNA
caacgccgcgtatgccttaaatcccgcttaccgcatcgagatgcgtcgacggctgagtacgctatacgacctacgcgacatcgcgtgtaggcgaaacaaccgtataacgaagcgcggctaagattcgcatgaccggccgaacctgatcag

SEQ ID NO: 11 - DNA, 150 bp, synthetic construct
  source 1..150: mol_type = other DNA
gcggcgaattgcaaacgtcgtcctcgggcgtaatacacgatacgtcccgaacgagaccgtgctacttaggcgcgtagcgagaacgcgtgtaccgaggatgcgattagatcgatccacgcgctgacgccgtcgatagtcgtatgcgtccag

SEQ ID NO: 12 - DNA, 150 bp, synthetic construct
  source 1..150: mol_type = other DNA
acacgcgtggagcgcgaattgtgatgcggacgctcgtatccgcggaaacgttcgatagggagtcgtgagcgtgcgacgtaagcgatgtgcgttatgccgtattccgtgcccgaataggaggcgcacgatttgtcgtacgctgctgcgcag

SEQ ID NO: 13 - DNA, 150 bp, synthetic construct
  source 1..150: mol_type = other DNA
ttgacggacgctgtcgcactaaacgtcgcgacgttactccgaactaatccgcacccgcgatgatcgcgctccaattccgttaatacgtccaccggcgcgagacgatagtacgagtcggcttgattgcgcgccgccaataccattcgacag

SEQ ID NO: 14 - DNA, 150 bp, synthetic construct
  source 1..150: mol_type = other DNA
gggcccgcgacttatatcgtgaccgtcgtactactcccgtccgctgatcaccgccgtaatcatcgaacgatcgagttggctcgtagtccaatcgacccgaagttgtcgccgaattgcgagtcgttctatcggaccggatctgtatcacag

SEQ ID NO: 15 - DNA, 150 bp, synthetic construct
  source 1..150: mol_type = other DNA
acaatcgcggcgtcacgttaagcgctatttccggatcgggccgaatgttccgtaccgacgaccgatgcacgtgcgatatgagcgcacggacgtacgagtttctaccgcgcgaaagcgtaagatgtacgcgtcgtaacgcttactagtcag

SEQ ID NO: 16 - DNA, 50 bp, synthetic construct
  source 1..50: mol_type = other DNA
ggagcggaccgtatgtcgacgtcgttaacgactcgccgtacggacatacg

SEQ ID NO: 17 - DNA, 50 bp, synthetic construct
  source 1..50: mol_type = other DNA
ggttatagcgcgcgttgttccgattcgcctcgcgtacgttactggcggat

SEQ ID NO: 18 - DNA, 50 bp, synthetic construct
  source 1..50: mol_type = other DNA
ggcggcattgtccgcgtaactcggtcgcggatatggtgtgcgcacgacgt

SEQ ID NO: 19 - DNA, 50 bp, synthetic construct
  source 1..50: mol_type = other DNA
ggaccgctattcgcgaccatatctcgcgcttaacgcgcgtccatagttgc

SEQ ID NO: 20 - DNA, 50 bp, synthetic construct
  source 1..50: mol_type = other DNA
ggactcgtctaccaatgcgcggtcgcacgaatataacgcgaccggacagc

SEQ ID NO: 21 - DNA, 50 bp, synthetic construct
  source 1..50: mol_type = other DNA
ggcgctacacggaacgctcatcgaatcgccggccgataacgttcctattg

SEQ ID NO: 22 - DNA, 50 bp, synthetic construct
  source 1..50: mol_type = other DNA
ggcgtcattacggcaccgtacttcggacgcggacaattcgaatagtcggc

SEQ ID NO: 23 - DNA, 50 bp, synthetic construct
  source 1..50: mol_type = other DNA
ggagccggttcggatcgcatatcgctaatcgcggagcacgtagtcgcgat

SEQ ID NO: 24 - DNA, 50 bp, synthetic construct
  source 1..50: mol_type = other DNA
ggaagcagcgcggttgtaacgacgcgacggtccgaatatagatcgcacgg

SEQ ID NO: 25 - DNA, 50 bp, synthetic construct
  source 1..50: mol_type = other DNA
ggctgatatacacggcgcacgtcgcgttatacggccggatatcggaacac

SEQ ID NO: 26 - DNA, 50 bp, synthetic construct
  source 1..50: mol_type = other DNA
ggccggatccgtcgcaatacgatgactggccgtctatagcgtgtacggcg

SEQ ID NO: 27 - DNA, 50 bp, synthetic construct
  source 1..50: mol_type = other DNA
ggatcgcgacctaacctcgatcgaagaccgcacgtaacggtatagtccgg

SEQ ID NO: 28 - DNA, 50 bp, synthetic construct
  source 1..50: mol_type = other DNA
ggagcacttgcgtactcgaccggtatacgccataacggtctatcacgcct

SEQ ID NO: 29 - DNA, 50 bp, synthetic construct
  source 1..50: mol_type = other DNA
ggattccggacgtcgtacgtctatccgccgaatgacggtcgagcgacctt

SEQ ID NO: 30 - DNA, 50 bp, synthetic construct
  source 1..50: mol_type = other DNA
ggtacaatccactcgatccgacggcggatgcaacgtacgtgacgaagtgc

SEQ ID NO: 31 - DNA, 333 bp, synthetic construct
  source 1..333: mol_type = other DNA
ccgcgtacccgtgatgactatcgcgccgttataccacgcaacggccatgctacgacgtatagttcgcacgcgatactcgaggcgttgcgccattgacgtttcgcgtggcgctattagtccgatccgcgacgactagtagcgtagagacaggtaagtatcaaggttacaagacaggtttaaggagaccaatagaaactgggcttgtcgagacagagaagactcttgcgtttctgataggcacctattggtcttactgacatccactttgcctttctctccacagggagcggaccgtatgtcgacgtcgttaacgactcgccgtacggacatacg

SEQ ID NO: 32 - DNA, 333 bp, synthetic construct
  source 1..333: mol_type = other DNA
tcgtcgctacgaacgcaacgtagcgcgacataccggcaatgcccgtaaacggcatcgtatagcgaatccgatcagtcgtcctgttaccgacgcgcaatactacccgcgtcactatacgctttagacgcctcgccgttactttattcgcaggtaagtatcaaggttacaagacaggtttaaggagaccaatagaaactgggcttgtcgagacagagaagactcttgcgtttctgataggcacctattggtcttactgacatccactttgcctttctctccacagggttatagcgcgcgttgttccgattcgcctcgcgtacgttactggcggat

SEQ ID NO: 33 - DNA, 333 bp, synthetic construct
  source 1..333: mol_type = other DNA
cgcaaagccgaagttacgcggattgtcgacccgcggctttcggacatttcgcgccgactatcgttcggcgctcgttattcgtaggcgtaatgccgagttgcgaacgacgcaagtacgcctaacgcccgtctaccgtacgtgtcgccgcaggtaagtatcaaggttacaagacaggtttaaggagaccaatagaaactgggcttgtcgagacagagaagactcttgcgtttctgataggcacctattggtcttactgacatccactttgcctttctctccacagggcggcattgtccgcgtaactcggtcgcggatatggtgtgcgcacgacgt

SEQ ID NO: 34 - DNA, 333 bp, synthetic construct
  source 1..333: mol_type = other DNA
cagcggaacgcgtacagtagccgtatgcgcgtcgcttagacgtttggcgaacgaactcgagtaacacgttcgcgttgaccgattcgtggcgcatcgcctaataatgcgtagtcggcggcgagttgtcgacgcgcccaatatctatgacaggtaagtatcaaggttacaagacaggtttaaggagaccaatagaaactgggcttgtcgagacagagaagactcttgcgtttctgataggcacctattggtcttactgacatccactttgcctttctctccacagggaccgctattcgcgaccatatctcgcgcttaacgcgcgtccatagttgc

SEQ ID NO: 35 - DNA, 333 bp, synthetic construct
  source 1..333: mol_type = other DNA
cacggaccactaatcgggaccgcagacgaacccgttcgaacaagcgtcgtcggagtaacccacgcgaattcgatggccgaacgttgacgacgctcgacattacgctgcgcgacgtattgtgcgtagcgtaagtcgtttcgtacacggcaggtaagtatcaaggttacaagacaggtttaaggagaccaatagaaactgggcttgtcgagacagagaagactcttgcgtttctgataggcacctattggtcttactgacatccactttgcctttctctccacagggactcgtctaccaatgcgcggtcgcacgaatataacgcgaccggacagc

SEQ ID NO: 36 - DNA, 333 bp, synthetic construct
  source 1..333: mol_type = other DNA
cgcgtacttccgactaaccgttccgtaacatacgcccgagcggcgcactacgatatagactggcgcgatcgtccatcgatgtagcgcgtggatgcatcgtttagctcgacaccggcgtgtgtcgaacgtcgcataacggacccgttgcaggtaagtatcaaggttacaagacaggtttaaggagaccaatagaaactgggcttgtcgagacagagaagactcttgcgtttctgataggcacctattggtcttactgacatccactttgcctttctctccacagggcgctacacggaacgctcatcgaatcgccggccgataacgttcctattg

SEQ ID NO: 37 - DNA, 333 bp, synthetic construct
  source 1..333: mol_type = other DNA
tctagtgcgacgcgaacgtttgccgtaccgtagacgagacccgttctacgatcgcctatcgatccggcataccgagagtcctcgtcgcagtacgcactttctcggcgcgattgtagcgttgtaatcgcgtgcgggcgaatagtggcgcaggtaagtatcaaggttacaagacaggtttaaggagaccaatagaaactgggcttgtcgagacagagaagactcttgcgtttctgataggcacctattggtcttactgacatccactttgcctttctctccacagggcgtcattacggcaccgtacttcggacgcggacaattcgaatagtcggc

SEQ ID NO: 38 - DNA, 333 bp, synthetic construct
  source 1..333: mol_type = other DNA
ctgttcgtaccacacgtcgagtccgcgtgatacgtttcgacgatctatacgcgcgccacttggacgcgtttaacgcccaccgagtacgattacgccggacttcgcgatatgcggacatcgaagcgtgcgtccgtatcgagcataaagcaggtaagtatcaaggttacaagacaggtttaaggagaccaatagaaactgggcttgtcgagacagagaagactcttgcgtttctgataggcacctattggtcttactgacatccactttgcctttctctccacagggagccggttcggatcgcatatcgctaatcgcggagcacgtagtcgcgat

SEQ ID NO: 39 - DNA, 333 bp, synthetic construct
  source 1..333: mol_type = other DNA
cgctgatacgacggataccgaccattactccgcgaggcgtcgcccgattagtgcatacggcgacccgccgacatcgttaagacgcaaattcgcgctacgggatgagcgacagcgttgcgaagtacgtccggagtcgtagataacggccaggtaagtatcaaggttacaagacaggtttaaggagaccaatagaaactgggcttgtcgagacagagaagactcttgcgtttctgataggcacctattggtcttactgacatccactttgcctttctctccacagggaagcagcgcggttgtaacgacgcgacggtccgaatatagatcgcacgg

SEQ ID NO: 40 - DNA, 333 bp, synthetic construct
  source 1..333: mol_type = other DNA
caacgccgcgtatgccttaaatcccgcttaccgcatcgagatgcgtcgacggctgagtacgctatacgacctacgcgacatcgcgtgtaggcgaaacaaccgtataacgaagcgcggctaagattcgcatgaccggccgaacctgatcaggtaagtatcaaggttacaagacaggtttaaggagaccaatagaaactgggcttgtcgagacagagaagactcttgcgtttctgataggcacctattggtcttactgacatccactttgcctttctctccacagggctgatatacacggcgcacgtcgcgttatacggccggatatcggaacac

SEQ ID NO: 41 - DNA, 333 bp, synthetic construct
  source 1..333: mol_type = other DNA
gcggcgaattgcaaacgtcgtcctcgggcgtaatacacgatacgtcccgaacgagaccgtgctacttaggcgcgtagcgagaacgcgtgtaccgaggatgcgattagatcgatccacgcgctgacgccgtcgatagtcgtatgcgtccaggtaagtatcaaggttacaagacaggtttaaggagaccaatagaaactgggcttgtcgagacagagaagactcttgcgtttctgataggcacctattggtcttactgacatccactttgcctttctctccacagggccggatccgtcgcaatacgatgactggccgtctatagcgtgtacggcg

SEQ ID NO: 42 - DNA, 333 bp, synthetic construct
  source 1..333: mol_type = other DNA
acacgcgtggagcgcgaattgtgatgcggacgctcgtatccgcggaaacgttcgatagggagtcgtgagcgtgcgacgtaagcgatgtgcgttatgccgtattccgtgcccgaataggaggcgcacgatttgtcgtacgctgctgcgcaggtaagtatcaaggttacaagacaggtttaaggagaccaatagaaactgggcttgtcgagacagagaagactcttgcgtttctgataggcacctattggtcttactgacatccactttgcctttctctccacagggatcgcgacctaacctcgatcgaagaccgcacgtaacggtatagtccgg

SEQ ID NO: 43 - DNA, 333 bp, synthetic construct
  source 1..333: mol_type = other DNA
ttgacggacgctgtcgcactaaacgtcgcgacgttactccgaactaatccgcacccgcgatgatcgcgctccaattccgttaatacgtccaccggcgcgagacgatagtacgagtcggcttgattgcgcgccgccaataccattcgacaggtaagtatcaaggttacaagacaggtttaaggagaccaatagaaactgggcttgtcgagacagagaagactcttgcgtttctgataggcacctattggtcttactgacatccactttgcctttctctccacagggagcacttgcgtactcgaccggtatacgccataacggtctatcacgcct

SEQ ID NO: 44 - DNA, 333 bp, synthetic construct
  source 1..333: mol_type = other DNA
gggcccgcgacttatatcgtgaccgtcgtactactcccgtccgctgatcaccgccgtaatcatcgaacgatcgagttggctcgtagtccaatcgacccgaagttgtcgccgaattgcgagtcgttctatcggaccggatctgtatcacaggtaagtatcaaggttacaagacaggtttaaggagaccaatagaaactgggcttgtcgagacagagaagactcttgcgtttctgataggcacctattggtcttactgacatccactttgcctttctctccacagggattccggacgtcgtacgtctatccgccgaatgacggtcgagcgacctt

SEQ ID NO: 45 - DNA, 333 bp, synthetic construct
  source 1..333: mol_type = other DNA
acaatcgcggcgtcacgttaagcgctatttccggatcgggccgaatgttccgtaccgacgaccgatgcacgtgcgatatgagcgcacggacgtacgagtttctaccgcgcgaaagcgtaagatgtacgcgtcgtaacgcttactagtcaggtaagtatcaaggttacaagacaggtttaaggagaccaatagaaactgggcttgtcgagacagagaagactcttgcgtttctgataggcacctattggtcttactgacatccactttgcctttctctccacagggtacaatccactcgatccgacggcggatgcaacgtacgtgacgaagtgc

SEQ ID NO: 46 - DNA, 20 bp, synthetic construct
  source 1..20: mol_type = other DNA
gtagagacaggtaagtatca

SEQ ID NO: 47 - DNA, 20 bp, synthetic construct
  source 1..20: mol_type = other DNA
aagtatcaaggttacaagac

SEQ ID NO: 48 - DNA, 20 bp, synthetic construct
  source 1..20: mol_type = other DNA
tacaagacaggtttaaggag

SEQ ID NO: 49 - DNA, 20 bp, synthetic construct
  source 1..20: mol_type = other DNA
tttctgataggcacctattg

SEQ ID NO: 50 - DNA, 20 bp, synthetic construct
  source 1..20: mol_type = other DNA
ctctccacagggagcggacc

SEQ ID NO: 51 - DNA, 20 bp, synthetic construct
  source 1..20: mol_type = other DNA
tacgctttagacgcctcgcc

SEQ ID NO: 52 - DNA, 20 bp, synthetic construct
  source 1..20: mol_type = other DNA
ttattcgcaggtaagtatca

SEQ ID NO: 53 - DNA, 20 bp, synthetic construct
  source 1..20: mol_type = other DNA
ttattcgcaggtaagtatca

SEQ ID NO: 54 - DNA, 20 bp, synthetic construct
  source 1..20: mol_type = other DNA
aagtatcaaggttacaagac

SEQ ID NO: 55 - DNA, 20 bp, synthetic construct
  source 1..20: mol_type = other DNA
tacaagacaggtttaaggag

SEQ ID NO: 56 - DNA, 20 bp, synthetic construct
  source 1..20: mol_type = other DNA
tttctgataggcacctattg

SEQ ID NO: 57 - DNA, 20 bp, synthetic construct
  source 1..20: mol_type = other DNA
ctctccacagggttatagcg

SEQ ID NO: 58 - DNA, 20 bp, synthetic construct
  source 1..20: mol_type = other DNA
ttattcgtaggcgtaatgcc

SEQ ID NO: 59 - DNA, 20 bp, synthetic construct
  source 1..20: mol_type = other DNA
cccgtctaccgtacgtgtcg

SEQ ID NO: 60 - DNA, 20 bp, synthetic construct
  source 1..20: mol_type = other DNA
gtcgccgcaggtaagtatca

SEQ ID NO: 61 - DNA, 20 bp, synthetic construct
  source 1..20: mol_type = other DNA
gtcgccgcaggtaagtatca

SEQ ID NO: 62 - DNA, 20 bp, synthetic construct
  source 1..20: mol_type = other DNA
aagtatcaaggttacaagac

SEQ ID NO: 63 - DNA, 20 bp, synthetic construct
  source 1..20: mol_type = other DNA
tacaagacaggtttaaggag

SEQ ID NO: 64 - DNA, 20 bp, synthetic construct
  source 1..20: mol_type = other DNA
tttctgataggcacctattg

SEQ ID NO: 65 - DNA, 20 bp, synthetic construct
  source 1..20: mol_type = other DNA
ctctccacagggcggcattg

SEQ ID NO: 66 - DNA, 20 bp, synthetic construct
  source 1..20: mol_type = other DNA
acgaactcgagtaacacgtt

SEQ ID NO: 67 - DNA, 20 bp, synthetic construct
  source 1..20: mol_type = other DNA
tctatgacaggtaagtatca

SEQ ID NO: 68 - DNA, 20 bp, synthetic construct
  source 1..20: mol_type = other DNA
tctatgacaggtaagtatca

SEQ ID NO: 69 - DNA, 20 bp, synthetic construct
  source 1..20: mol_type = other DNA
aagtatcaaggttacaagac

SEQ ID NO: 70 - DNA, 20 bp, synthetic construct
  source 1..20: mol_type = other DNA
tacaagacaggtttaaggag

SEQ ID NO: 71 - DNA, 20 bp, synthetic construct
  source 1..20: mol_type = other DNA
tttctgataggcacctattg

SEQ ID NO: 72 - DNA, 20 bp, synthetic construct
  source 1..20: mol_type = other DNA
ctctccacagggaccgctat

SEQ ID NO: 73 - DNA, 20 bp, synthetic construct
  source 1..20: mol_type = other DNA
gcgacgtattgtgcgtagcg

SEQ ID NO: 74 - DNA, 20 bp, synthetic construct
  source 1..20: mol_type = other DNA
tgtgcgtagcgtaagtcgtt

SEQ ID NO: 75 - DNA, 20 bp, synthetic construct
  source 1..20: mol_type = other DNA
tacacggcaggtaagtatca

SEQ ID NO: 76 - DNA, 20 bp, synthetic construct
  source 1..20: mol_type = other DNA
tacacggcaggtaagtatca

SEQ ID NO: 77 - DNA, 20 bp, synthetic construct
  source 1..20: mol_type = other DNA
aagtatcaaggttacaagac

SEQ ID NO: 78 - DNA, 20 bp, synthetic construct
  source 1..20: mol_type = other DNA
tacaagacaggtttaaggag

SEQ ID NO: 79 - DNA, 20 bp, synthetic construct
  source 1..20: mol_type = other DNA
tttctgataggcacctattg

SEQ ID NO: 80 - DNA, 20 bp, synthetic construct
  source 1..20: mol_type = other DNA
ctctccacagggactcgtct

SEQ ID NO: 81 - DNA, 20 bp, synthetic construct
  source 1..20: mol_type = other DNA
cccgttgcaggtaagtatca

SEQ ID NO: 82 - DNA, 20 bp, synthetic construct
  source 1..20: mol_type = other DNA
cccgttgcaggtaagtatca

SEQ ID NO: 83 - DNA, 20 bp, synthetic construct
  source 1..20: mol_type = other DNA
aagtatcaaggttacaagac

SEQ ID NO: 84 - DNA, 20 bp, synthetic construct
  source 1..20: mol_type = other DNA
tacaagacaggtttaaggag

SEQ ID NO: 85 - DNA, 20 bp, synthetic construct
  source 1..20: mol_type = other DNA
tttctgataggcacctattg

SEQ ID NO: 86 - DNA, 20 bp, synthetic construct
  source 1..20: mol_type = other DNA
ctctccacagggcgctacac

SEQ ID NO: 87 - DNA, 20 bp, synthetic construct
  source 1..20: mol_type = other DNA
cctcgtcgcagtacgcactt

SEQ ID NO: 88 - DNA, 20 bp, synthetic construct
  source 1..20: mol_type = other DNA
ctcgtcgcagtacgcacttt

SEQ ID NO: 89 - DNA, 20 bp, synthetic construct
  source 1..20: mol_type = other DNA
agtggcgcaggtaagtatca

SEQ ID NO: 90 - DNA, 20 bp, synthetic construct
  source 1..20: mol_type = other DNA
aagtatcaaggttacaagac

SEQ ID NO: 91 - DNA, 20 bp, synthetic construct
  source 1..20: mol_type = other DNA
tacaagacaggtttaaggag

SEQ ID NO: 92 - DNA, 20 bp, synthetic construct
  source 1..20: mol_type = other DNA
tttctgataggcacctattg

SEQ ID NO: 93 - DNA, 20 bp, synthetic construct
  source 1..20: mol_type = other DNA
ctctccacagggcgtcatta

SEQ ID NO: 94 - DNA, 20 bp, synthetic construct
  source 1..20: mol_type = other DNA
cataaagcaggtaagtatca

SEQ ID NO: 95 - DNA, 20 bp, synthetic construct
  source 1..20: mol_type = other DNA
aagtatcaaggttacaagac

SEQ ID NO: 96 - DNA, 20 bp, synthetic construct
  source 1..20: mol_type = other DNA
tacaagacaggtttaaggag

SEQ ID NO: 97 - DNA, 20 bp, synthetic construct
  source 1..20: mol_type = other DNA
tttctgataggcacctattg

SEQ ID NO: 98 - DNA, 20 bp, synthetic construct
  source 1..20: mol_type = other DNA
ctctccacagggagccggtt

SEQ ID NO: 99 - DNA, 20 bp, synthetic construct
  source 1..20: mol_type = other DNA
gcgttgcgaagtacgtccgg

SEQ ID NO: 100 - DNA, 20 bp, synthetic construct
  source 1..20: mol_type = other DNA
taacggccaggtaagtatca

SEQ ID NO: 101 - DNA, 20 bp, synthetic construct
  source 1..20: mol_type = other DNA
aagtatcaaggttacaagac

SEQ ID NO: 102 - DNA, 20 bp, synthetic construct
  source 1..20: mol_type = other DNA
tacaagacaggtttaaggag

SEQ ID NO: 103 - DNA, 20 bp, synthetic construct
  source 1..20: mol_type = other DNA
tttctgataggcacctattg

SEQ ID NO: 104 - DNA, 20 bp, synthetic construct
  source 1..20: mol_type = other DNA
ctctccacagggaagcagcg

SEQ ID NO: 105 - DNA, 20 bp, synthetic construct
  source 1..20: mol_type = other DNA
acctgatcaggtaagtatca

SEQ ID NO: 106 - DNA, 20 bp, synthetic construct
  source 1..20: mol_type = other DNA
gatcaggtaagtatcaaggt

SEQ ID NO: 107 - DNA, 20 bp, synthetic construct
  source 1..20: mol_type = other DNA
aagtatcaaggttacaagac

SEQ ID NO: 108 - DNA, 20 bp, synthetic construct
  source 1..20: mol_type = other DNA
tacaagacaggtttaaggag

SEQ ID NO: 109 - DNA, 20 bp, synthetic construct
  source 1..20: mol_type = other DNA
tttctgataggcacctattg

SEQ ID NO: 110 - DNA, 20 bp, synthetic construct
  source 1..20: mol_type = other DNA
ctctccacagggctgatata

SEQ ID NO: 111 - DNA, 20 bp, synthetic construct
  source 1..20: mol_type = other DNA
atgcgtccaggtaagtatca

SEQ ID NO: 112 - DNA, 20 bp, synthetic construct
  source 1..20: mol_type = other DNA
atgcgtccaggtaagtatca

SEQ ID NO: 113 - DNA, 20 bp, synthetic construct
  source 1..20: mol_type = other DNA
aagtatcaaggttacaagac

SEQ ID NO: 114 - DNA, 20 bp, synthetic construct
  source 1..20: mol_type = other DNA
tacaagacaggtttaaggag

SEQ ID NO: 115 - DNA, 20 bp, synthetic construct
  source 1..20: mol_type = other DNA
tttctgataggcacctattg

SEQ ID NO: 116 - DNA, 20 bp, synthetic construct
  source 1..20: mol_type = other DNA
ctctccacagggccggatcc

SEQ ID NO: 117 - DNA, 20 bp, synthetic construct
  source 1..20: mol_type = other DNA
agcgtgcgacgtaagcgatg

SEQ ID NO: 118 - DNA, 20 bp, synthetic construct
  source 1..20: mol_type = other DNA
cgtaagcgatgtgcgttatg

SEQ ID NO: 119 - DNA, 20 bp, synthetic construct
  source 1..20: mol_type = other DNA
gcccgaataggaggcgcacg

SEQ ID NO: 120 - DNA, 20 bp, synthetic construct
  source 1..20: mol_type = other DNA
tgctgcgcaggtaagtatca

SEQ ID NO: 121 - DNA, 20 bp, synthetic construct
  source 1..20: mol_type = other DNA
tgctgcgcaggtaagtatca

SEQ ID NO: 122 - DNA, 20 bp, synthetic construct
  source 1..20: mol_type = other DNA
aagtatcaaggttacaagac

SEQ ID NO: 123 - DNA, 20 bp, synthetic construct
  source 1..20: mol_type = other DNA
tacaagacaggtttaaggag

SEQ ID NO: 124 - DNA, 20 bp, synthetic construct
  source 1..20: mol_type = other DNA
tttctgataggcacctattg

SEQ ID NO: 125 - DNA, 20 bp, synthetic construct
  source 1..20: mol_type = other DNA
ctctccacagggatcgcgac

SEQ ID NO: 126 - DNA, 20 bp, synthetic construct
  source 1..20: mol_type = other DNA
cattcgacaggtaagtatca

SEQ ID NO: 127 - DNA, 20 bp, synthetic construct
  source 1..20: mol_type = other DNA
cattcgacaggtaagtatca

SEQ ID NO: 128 - DNA, 20 bp, synthetic construct
  source 1..20: mol_type = other DNA
aagtatcaaggttacaagac

SEQ ID NO: 129 - DNA, 20 bp, synthetic construct
  source 1..20: mol_type = other DNA
tacaagacaggtttaaggag

SEQ ID NO: 130 - DNA, 20 bp, synthetic construct
  source 1..20: mol_type = other DNA
tttctgataggcacctattg

SEQ ID NO: 131 - DNA, 20 bp, synthetic construct
  source 1..20: mol_type = other DNA
ctctccacagggagcacttg

SEQ ID NO: 132 - DNA, 20 bp, synthetic construct
  source 1..20: mol_type = other DNA
tgtatcacaggtaagtatca

SEQ ID NO: 133 - DNA, 20 bp, synthetic construct
  source 1..20: mol_type = other DNA
tgtatcacaggtaagtatca

SEQ ID NO: 134 - DNA, 20 bp, synthetic construct
  source 1..20: mol_type = other DNA
aagtatcaaggttacaagac

SEQ ID NO: 135 - DNA, 20 bp, synthetic construct
  source 1..20: mol_type = other DNA
tacaagacaggtttaaggag

SEQ ID NO: 136 - DNA, 20 bp, synthetic construct
  source 1..20: mol_type = other DNA
tttctgataggcacctattg

SEQ ID NO: 137 - DNA, 20 bp, synthetic construct
  source 1..20: mol_type = other DNA
ctctccacagggattccgga

SEQ ID NO: 138 - DNA, 20 bp, synthetic construct
  source 1..20: mol_type = other DNA
gcgcgaaagcgtaagatgta

SEQ ID NO: 139 - DNA, 20 bp, synthetic construct
  source 1..20: mol_type = other DNA
agcgtaagatgtacgcgtcg

SEQ ID NO: 140 - DNA, 20 bp, synthetic construct
  source 1..20: mol_type = other DNA
tactagtcaggtaagtatca

SEQ ID NO: 141 - DNA, 20 bp, synthetic construct
  source 1..20: mol_type = other DNA
tactagtcaggtaagtatca

SEQ ID NO: 142 - DNA, 20 bp, synthetic construct
  source 1..20: mol_type = other DNA
aagtatcaaggttacaagac

SEQ ID NO: 143 - DNA, 20 bp, synthetic construct
  source 1..20: mol_type = other DNA
tacaagacaggtttaaggag

SEQ ID NO: 144 - DNA, 20 bp, synthetic construct
  source 1..20: mol_type = other DNA
tttctgataggcacctattg

SEQ ID NO: 145 - DNA, 20 bp, synthetic construct
  source 1..20: mol_type = other DNA
ctctccacagggtacaatcc

SEQ ID NO: 146 - DNA, 21 bp, synthetic construct
  source 1..21: mol_type = other DNA
ggccatgctacgacgtatagt

SEQ ID NO: 147 - DNA, 19 bp, synthetic construct
  source 1..19: mol_type = other DNA
gcggctttcggacatttcg

SEQ ID NO: 148 - DNA, 21 bp, synthetic construct
  source 1..21: mol_type = other DNA
gagtaacacgttcgcgttgac

SEQ ID NO: 149 - DNA, 19 bp, synthetic construct
  source 1..19: mol_type = other DNA
aagcgtcgtcggagtaacc

SEQ ID NO: 150 - DNA, 21 bp, synthetic construct
  source 1..21: mol_type = other DNA
cggcgcactacgatatagact

SEQ ID NO: 151 - DNA, 19 bp, synthetic construct
  source 1..19: mol_type = other DNA
tcgtcgcagtacgcacttt

SEQ ID NO: 152 - DNA, 17 bp, synthetic construct
  source 1..17: mol_type = other DNA
gccacttggacgcgttt

SEQ ID NO: 153 - DNA, 20 bp, synthetic construct
  source 1..20: mol_type = other DNA
tcgcccgattagtgcatacg

SEQ ID NO: 154 - DNA, 19 bp, synthetic construct
  source 1..19: mol_type = other DNA
cgcgtgtaggcgaaacaac

SEQ ID NO: 155 - DNA, 18 bp, synthetic construct
  source 1..18: mol_type = other DNA
ccgaacgagaccgtgcta

SEQ ID NO: 156 - DNA, 19 bp, synthetic construct
  source 1..19: mol_type = other DNA
cgcggaaacgttcgatagg

SEQ ID NO: 157 - DNA, 23 bp, synthetic construct
  source 1..23: mol_type = other DNA
gcgacgttactccgaactaatcc

SEQ ID NO: 158 - DNA, 19 bp, synthetic construct
  source 1..19: mol_type = other DNA
ctactcccgtccgctgatc

SEQ ID NO: 159 - DNA, 18 bp, synthetic construct
  source 1..18: mol_type = other DNA
gtaccgacgaccgatgca

SEQ ID NO: 160 - DNA, 15 bp, synthetic construct
  source 1..15: mol_type = other DNA
tcgcacgcgatactc

SEQ ID NO: 161 - DNA, 15 bp, synthetic construct
  source 1..15: mol_type = other DNA
tcggcgctcgttatt

SEQ ID NO: 162 - DNA, 15 bp, synthetic construct
  source 1..15: mol_type = other DNA
atgcgccacgaatcg

SEQ ID NO: 163 - DNA, 15 bp, synthetic construct
  source 1..15: mol_type = other DNA
tcggccatcgaattc

SEQ ID NO: 164 - DNA, 15 bp, synthetic construct
  source 1..15: mol_type = other DNA
ccacgcgctacatcg

SEQ ID NO: 165 - DNA, 16 bp, synthetic construct
  source 1..16: mol_type = other DNA
ctcggcgcgattgtag

SEQ ID NO: 166 - DNA, 16 bp, synthetic construct
  source 1..16: mol_type = other DNA
acgcccaccgagtacg

SEQ ID NO: 167 - DNA, 14 bp, synthetic construct
  source 1..14: mol_type = other DNA
acccgccgacatcg

SEQ ID NO: 168 - DNA, 16 bp, synthetic construct
  source 1..16: mol_type = other DNA
ccgcgcttcgttatac

SEQ ID NO: 169 - DNA, 17 bp, synthetic construct
  source 1..17: mol_type = other DNA
tctcgctacgcgcctaa

SEQ ID NO: 170 - DNA, 15 bp, synthetic construct
  source 1..15: mol_type = other DNA
acgtcgcacgctcac

SEQ ID NO: 171 - DNA, 16 bp, synthetic construct
  source 1..16: mol_type = other DNA
ttggagcgcgatcatc

SEQ ID NO: 172 - DNA, 15 bp, synthetic construct
  source 1..15: mol_type = other DNA
ccgccgtaatcatcg

SEQ ID NO: 173 - DNA, 16 bp, synthetic construct
  source 1..16: mol_type = other DNA
tccgtgcgctcatatc

SEQ ID NO: 174 - DNA, 19 bp, synthetic construct
  source 1..19: mol_type = other DNA
ccacgcgaaacgtcaatgg

SEQ ID NO: 175 - DNA, 20 bp, synthetic construct
  source 1..20: mol_type = other DNA
gttcgcaactcggcattacg

SEQ ID NO: 176 - DNA, 22 bp, synthetic construct
  source 1..22: mol_type = other DNA
ccgccgactacgcattattagg

SEQ ID NO: 177 - DNA, 17 bp, synthetic construct
  source 1..17: mol_type = other DNA
tcgagcgtcgtcaacgt

SEQ ID NO: 178 - DNA, 21 bp, synthetic construct
  source 1..21: mol_type = other DNA
ggtgtcgagctaaacgatgca

SEQ ID NO: 179 - DNA, 18 bp, synthetic construct
  source 1..18: mol_type = other DNA
cccgcacgcgattacaac

SEQ ID NO: 180 - DNA, 17 bp, synthetic construct
  source 1..17: mol_type = other DNA
cgcgaagtccggcgtaa

SEQ ID NO: 181 - DNA, 22 bp, synthetic construct
  source 1..22: mol_type = other DNA
gtagcgcgaatttgcgtcttaa

SEQ ID NO: 182 - DNA, 20 bp, synthetic construct
  source 1..20: mol_type = other DNA
gccggtcatgcgaatcttag

SEQ ID NO: 183 - DNA, 24 bp, synthetic construct
  source 1..24: mol_type = other DNA
gatctaatcgcatcctcggtacac

SEQ ID NO: 184 - DNA, 22 bp, synthetic construct
  source 1..22: mol_type = other DNA
ggaatacggcataacgcacatc

SEQ ID NO: 185 - DNA, 19 bp, synthetic construct
  source 1..19: mol_type = other DNA
gcgccggtggacgtattaa

SEQ ID NO: 186 - DNA, 20 bp, synthetic construct
  source 1..20: mol_type = other DNA
tcgggtcgattggactacga

SEQ ID NO: 187 - DNA, 19 bp, synthetic construct
  source 1..19: mol_type = other DNA
gcgcggtagaaactcgtac

SEQ ID NO: 188 - DNA, 20 bp, synthetic construct
  source 1..20: mol_type = other DNA
cgatccgcgacgactagtag

SEQ ID NO: 189 - DNA, 17 bp, synthetic construct
  source 1..17: mol_type = other DNA
gcccgtctaccgtacgt

SEQ ID NO: 190 - DNA, 18 bp, synthetic construct
  source 1..18: mol_type = other DNA
tgtcgacgcgcccaatat

SEQ ID NO: 191 - DNA, 22 bp, synthetic construct
  source 1..22: mol_type = other DNA
gcgtagcgtaagtcgtttcgta

SEQ ID NO: 192 - DNA, 20 bp, synthetic construct
  source 1..20: mol_type = other DNA
gtgtcgaacgtcgcataacg

SEQ ID NO: 193 - DNA, 14 bp, synthetic construct
  source 1..14: mol_type = other DNA
gcgtgcgggcgaat

SEQ ID NO: 194 - DNA, 17 bp, synthetic construct
  source 1..17: mol_type = other DNA
gcgtgcgtccgtatcga

SEQ ID NO: 195 - DNA, 19 bp, synthetic construct
  source 1..19: mol_type = other DNA
acagcgttgcgaagtacgt

SEQ ID NO: 196 - DNA, 17 bp, synthetic construct
  source 1..17: mol_type = other DNA
catgaccggccgaacct

SEQ ID NO: 197 - DNA, 19 bp, synthetic construct
  source 1..19: mol_type = other DNA
gacgccgtcgatagtcgta

SEQ ID NO: 198 - DNA, 18 bp, synthetic construct
  source 1..18: mol_type = other DNA
ggaggcgcacgatttgtc

SEQ ID NO: 199 - DNA, 15 bp, synthetic construct
  source 1..15: mol_type = other DNA
cgcgccgccaatacc

SEQ ID NO: 200 - DNA, 22 bp, synthetic construct
  source 1..22: mol_type = other DNA
cgaattgcgagtcgttctatcg

SEQ ID NO: 201 - DNA, 21 bp, synthetic construct
  source 1..21: mol_type = other DNA
gcgcgaaagcgtaagatgtac

SEQ ID NO: 202 - DNA, 17 bp, synthetic construct
  source 1..17: mol_type = other DNA
tccgctccctgtctcta

SEQ ID NO: 203 - DNA, 15 bp, synthetic construct
  source 1..15: mol_type = other DNA
ccgccctgcggcgac

SEQ ID NO: 204 - DNA, 20 bp, synthetic construct
  source 1..20: mol_type = other DNA
aatagcggtccctgtcatag

SEQ ID NO: 205 - DNA, 16 bp, synthetic construct
  source 1..16: mol_type = other DNA
acggcagggactcgtc

SEQ ID NO: 206 - DNA, 16 bp, synthetic construct
  source 1..16: mol_type = other DNA
ttgcagggcgctacac

SEQ ID NO: 207 - DNA, 15 bp, synthetic construct
  source 1..15: mol_type = other DNA
acgccctgcgccact

SEQ ID NO: 208 - DNA, 16 bp, synthetic construct
  source 1..16: mol_type = other DNA
ccggctccctgcttta

SEQ ID NO: 209 - DNA, 17 bp, synthetic construct
  source 1..17: mol_type = other DNA
cttccctggccgttatc

SEQ ID NO: 210 - DNA, 21 bp, synthetic construct
  source 1..21: mol_type = other DNA
ccgtgtatatcagccctgatc

SEQ ID NO: 211 - DNA, 16 bp, synthetic construct
  source 1..16: mol_type = other DNA
tccggccctggacgca

SEQ ID NO: 212 - DNA, 18 bp, synthetic construct
  source 1..18: mol_type = other DNA
cgcagggatcgcgaccta

SEQ ID NO: 213 - DNA, 17 bp, synthetic construct
  source 1..17: mol_type = other DNA
tcgacagggagcacttg

SEQ ID NO: 214 - DNA, 19 bp, synthetic construct
  source 1..19: mol_type = other DNA
ccggaatccctgtgataca

SEQ ID NO: 215 - DNA, 19 bp, synthetic construct
  source 1..19: mol_type = other DNA
tagtcagggtacaatccac

SEQ ID NO: 216 - DNA, 21 bp, synthetic construct
  source 1..21: mol_type = other DNA
cgttaacgacgtcgacatacg

SEQ ID NO: 217 - DNA, 20 bp, synthetic construct
  source 1..20: mol_type = other DNA
accgagttacgcggacaatg

SEQ ID NO: 218 - DNA, 21 bp, synthetic construct
  source 1..21: mol_type = other DNA
gcgttaagcgcgagatatggt

SEQ ID NO: 219 - DNA, 15 bp, synthetic construct
  source 1..15: mol_type = other DNA
gcgaccgcgcattgg

SEQ ID NO: 220 - DNA, 17 bp, synthetic construct
  source 1..17: mol_type = other DNA
gccggcgattcgatgag

SEQ ID NO: 221 - DNA, 18 bp, synthetic construct
  source 1..18: mol_type = other DNA
tgtccgcgtccgaagtac

SEQ ID NO: 222 - DNA, 20 bp, synthetic construct
  source 1..20: mol_type = other DNA
ctccgcgattagcgatatgc

SEQ ID NO: 223 - DNA, 18 bp, synthetic construct
  source 1..18: mol_type = other DNA
ccgtcgcgtcgttacaac

SEQ ID NO: 224 - DNA, 23 bp, synthetic construct
  source 1..23: mol_type = other DNA
gttccgatatccggccgtataac

SEQ ID NO: 225 - DNA, 19 bp, synthetic construct
  source 1..19: mol_type = other DNA
cggccagtcatcgtattgc

SEQ ID NO: 226 - DNA, 20 bp, synthetic construct
  source 1..20: mol_type = other DNA
cgttacgtgcggtcttcgat

SEQ ID NO: 227 - DNA, 20 bp, synthetic construct
  source 1..20: mol_type = other DNA
ggcgtataccggtcgagtac

SEQ ID NO: 228 - DNA, 21 bp, synthetic construct
  source 1..21: mol_type = other DNA
gtcattcggcggatagacgta

SEQ ID NO: 229 - DNA, 15 bp, synthetic construct
  source 1..15: mol_type = other DNA
ccgccgtcggatcga

SEQ ID NO: 230 - DNA, 19 bp, synthetic construct
  source 1..19: mol_type = other DNA
ccgttataccacgcaacgg

SEQ ID NO: 231 - DNA, 19 bp, Homo sapiens
  source 1..19: mol_type = genomic DNA
ccgttagaccacgcaacgg

SEQ ID NO: 232 - DNA, 19 bp, synthetic construct
  source 1..19: mol_type = other DNA
gacgactagtagcgtagag

SEQ ID NO: 233 - DNA, 19 bp, Homo sapiens
  source 1..19: mol_type = genomic DNA
gacgactagtagagtagag

SEQ ID NO: 234 - DNA, 16 bp, Canis lupus
  source 1..16: mol_type = genomic DNA
gtacccgtgatgacta

SEQ ID NO: 235 - DNA, 16 bp, Canis lupus
  source 1..16: mol_type = genomic DNA
ctcgaggcgttgcgcc

SEQ ID NO: 236 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag